SEQUENCE LISTING (210 sequences)

SEQ ID NO: 1 - protein, 393 aa, Artificial Sequence
  Synthetic construct - exemplary anti-EGFR hemi-COBRA (Pro6 - alpha-EGFR-1 sdAb - alpha-CD3 Vh - Flag - alpha-CD3Vli - His6)
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLHHHHHH

SEQ ID NO: 2 - DNA, 1182 bp, Artificial Sequence
  Synthetic construct - exemplary anti-EGFR hemi-COBRA (Pro6 - alpha-EGFR-1 sdAb - alpha-CD3 Vh - Flag - alpha-CD3Vli - His6)
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatgaactgggtacgacaagcccccggtaaagggcttgaatgggttgcaagaatacgcagtaaatacaataattatgcgacttattatgccgatagtgtaaaggaccgctttactatcagtagagatgacagtaagaacacggcttatttgcaaatgaacaacttgaagacagaagatacggcggtctattattgtgtacgacacggtaattttgggaattcatatataagctattgggcatactggggtcaaggaacccttgttacggtgagcagcgggggcggtggtgactataaggacgacgatgacaaaggcggcggctcccagactgtggtaacacaggaaccatctttgacagtaagtcctggaggtacggtcacgctcacttgtgggtcctcaaccggggctgtaacgtcaggcaattaccctaactgggtccaacagaagcctggacaagctcccaggggtctgataggcgattacaaagatgatgatgataagggcactccagcgcgctttagcggttctcttctgggtggaaaagcagccctcactctgagtggagtacaacccgaggatgaggcggaatattattgcgtgctctggtattcaaaccgctgggtcttcggtggcggtacgaaacttactgtactgcatcatcatcatcaccactga

SEQ ID NO: 3 - protein, 390 aa, Artificial Sequence
  Synthetic construct - exemplary anti-EGFR hemi-COBRA (Pro7 - alpha-CD3Vhi -  Flag - alpha-CD3Vl - alpha-EGFR-2 sdAb - His6)
EVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGDYKDDDDKGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGSQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSHHHHHH

SEQ ID NO: 4 - DNA, 1173 bp, Artificial Sequence
  Synthetic construct - exemplary anti-EGFR hemi-COBRA (Pro7 - alpha-CD3Vhi -  Flag - alpha-CD3Vl - alpha-EGFR-2 sdAb - His6)
gaggttcagcttgttgaatcagggggaggtctggtacagccaggcggaagtttgaaactgagttgtgcagcttctggatttacgttcaacaaatacgccatgaattgggtgagacaggcaccgggcaaggggcttgaatgggtcgcaaggatccggtccaagtacgactacaaggacgatgacgataaggctgactctgtaaaagaccgatttacaatatccagagacgattcaaaaaacactgcgtatctccagatgaacaatttgaaaacagaggatactgcggtttactattgtgtgagacacggcaacttcggcaacagctacatcagctattgggcctattggggacagggcactctcgtaacggtttcatccgggggaggaggagactacaaggacgatgacgataagggcggaggctctcagacggtcgtaactcaggagccatctctcactgttagcccgggcggaactgttactctcacctgtgggagcagtactggggcggttacttccggcaactaccctaactgggttcaacagaagccaggtcaggcaccaagaggtctgataggcggaactaaattcctcgcccctggtacccctgcacgattcagcggatcccttttgggcggcaaagcggctcttacactttctggagtccaaccggaagatgaggcggaatactattgtgtactttggtatagtaatcgctgggtattcggcggcggcaccaaactcactgtccttggaggaggaggaagcggcggaggttcccaggtcaagctggaggaatcgggtggaggctcagtgcagacaggaggtagcctccggctcacttgcgccgcttccggaaggacttcccggagctacgggatgggctggtttcggcaagcccccggaaaggagagagaattcgtgtccggaattagctggaggggcgactcaactggatacgcggactccgtcaagggcagattcactatctctcgggacaacgccaagaacaccgtggacttgcaaatgaattccctgaagccggaggacactgccatctactactgtgctgcggcagcaggatctgcctggtacggcaccctttatgaatacgattactggggacagggaacccaggtcacggtctcgagtcaccaccaccatcaccactga

SEQ ID NO: 5 - protein, 517 aa, Artificial Sequence
  Synthetic construct - exemplary anti-EGFR hemi-COBRA (Pro16 - alpha-EGFR-1 sdAb - alpha- CD3 Vh - Flag - alpha-CD3Vli - alpha-HSA - His6)
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGDYKDDDDKGGGSQTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 6 - DNA, 1554 bp, Artificial Sequence
  Synthetic construct - exemplary anti-EGFR hemi-COBRA (Pro16 - alpha-EGFR-1 sdAb - alpha- CD3 Vh - Flag - alpha-CD3Vli - alpha-HSA - His6)
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatcagtagagatgacagtaagaacacggcttatttgcaaatgaacaacttgaagacagaagatacggcggtctattattgtgtacgacacgcaaattttgggaattcatatataagctattgggcatactggggtcaaggaacccttgttacggtgagcagcgggggcggtggtgactataaggacgacgatgacaaaggcggcggctcccagactgtggtaacacaggaaccatctttgacagtaagtcctggaggtacggtcacgctcacttgtgggtcctcaaccggggctgtaacgtcaggcaattaccctaactgggtccaacagaagcctggacaagctcccaggggtctgataggcgattacaaagatgatgatgataagggcactccagcgcgctttagcggatcccttctgggtggaaaagcagccctcactctgagtggagtacaacccgaggatgaggcggaatattattgcgtgctctggtattcaaaccgctgggtcttcggtggcggtacgaaacttactgtactggggggaggcggctcaggcggcggatcagaagtgcagcttgttgaatctggcggaggtctggtccagccaggtaacagcttgagactgtcctgtgctgctagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccactga

SEQ ID NO: 7 - protein, 514 aa, Artificial Sequence
  Synthetic construct - exemplary anti-EGFR hemi-COBRA (Pro19 - alpha-EGFR-2 sdAb - alpha-CD3Vl ( 2B2) - Flag - alpha-CD3Vhi - alpha-HSA - His6)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGDYKDDDDKGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 8 - DNA, 1545 bp, Artificial Sequence
  Synthetic construct - exemplary anti-EGFR hemi-COBRA (Pro19 - alpha-EGFR-2 sdAb - alpha-CD3Vl ( 2B2) - Flag - alpha-CD3Vhi - alpha-HSA - His6)
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttggaggcggcggtgactacaaggacgacgatgacaaaggcggcggcagcgaggtccagttggtagaatccggaggtggattggttcaaccgggaggaagccttaagctttcatgcgccgcatccggattcaccttcaataagtacgcaatgaattgggttagacaggcaccaggtaaagggttggaatgggtggcacgcattaggtctaaatacgattacaaggacgacgacgacaaagctgacagcgtaaaagaccgatttacgataagccgggatgattctaagaacactgcttatttgcagatgaataatttgaagaccgaggatactgctgtctattattgcgtccgccacggtaattttggtaactcttacattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcactga

SEQ ID NO: 9 - protein, 515 aa, Artificial Sequence
  Synthetic construct - exemplary anti-EGFR hemi-COBRA (Pro39 - alpha-EGFR-1 sdAb - alpha- CD3 Vh - MMP9 - alpha-CD3VliGL - alpha-HSA - His6)
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGPGPAGMKGLPGSQTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 10 - DNA, 1548 bp, Artificial Sequence
  Synthetic construct - exemplary anti-EGFR hemi-COBRA (Pro39 - alpha-EGFR-1 sdAb - alpha- CD3 Vh - MMP9 - alpha-CD3VliGL - alpha-HSA - His6)
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatctctcgagatgactctaagaacactgcctatttgcagatgaacaatcttaaaacagaggacacagcggtgtactattgtgtaagacatgccaactttggaaacagctatattagctattgggcttactgggggcagggcactctggtcaccgtcagttcctctggggggccagggccagcgggcatgaaaggccttccgggatcccagactgtggtaacacaggaaccatctttgacagtaagtcctggaggtacggtcacgctcacttgtgggtcctcaaccggggctgtaacgtcaggccattaccctaactgggtccaacagaagcctggacaagctcccaggggtctgataggcggaacttcaaacaagcactcttggactccagcgcgctttagcggttcccttctgggtggaaaagcagccctcactctgagtggagtacaacccgaggatgaggcggaatattattgcgtgctctggggttcacgccgctgggtcttcggtggcggtacgaaacttactgtactggggggaggcggctcaggcggcggatcagaagtgcagcttgttgaatctggcggaggtctggtccagccaggtaacagcttgagactgtcctgtgctgcaagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccactga

SEQ ID NO: 11 - protein, 512 aa, Artificial Sequence
  Synthetic construct - exemplary anti-EGFR hemi-COBRA (Pro40 - alpha-EGFR-2 sdAb - alpha -CD3Vl - MMP9 - alpha-CD3VhiGL4 - alpha-HSA - His6)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 12 - DNA, 1539 bp, Artificial Sequence
  Synthetic construct - exemplary anti-EGFR hemi-COBRA (Pro40 - alpha-EGFR-2 sdAb - alpha -CD3Vl - MMP9 - alpha-CD3VhiGL4 - alpha-HSA - His6)
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtctttgggggtggtacgaagttgaccgttctcagcggtgggccaggaccagcaggtatgaaggggttgcccggctcagaagtccagttggtagaatccggggggggactggttcaaccaggaggtagtttgaagctttcatgcgccgcatccggattcaccttcagtgggtacgcaatgaattgggttagacaggcaccaggtaaagggttggaatgggtggcacgcattaggtccaaggccaacagctacgccaccgagtacgcggccagcgtaaaagaccgatttacgataagccgggatgattctaagaacactgcttatttgcagatgaataatttgaagaccgaggatactgctgtctattattgcgtccgccacggtaatgctggtaactctgccattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcactga

SEQ ID NO: 13 - protein, 515 aa, Artificial Sequence
  Synthetic construct - exemplary anti-EGFR hemi-COBRA (Pro41 - alpha-EGFR-1 sdAb - alpha- CD3 Vh - Meprin - alpha-CD3VliGL - alpha-HSA - His6)
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGGKKLADEPEGGSQTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 14 - DNA, 1548 bp, Artificial Sequence
  Synthetic construct - exemplary anti-EGFR hemi-COBRA (Pro41 - alpha-EGFR-1 sdAb - alpha- CD3 Vh - Meprin - alpha-CD3VliGL - alpha-HSA - His6)
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatctctcgagatgattctaaaaacaccgcatatttgcagatgaacaatcttaaaactgaggacaccgcagtgtattactgcgtgcgacatgcgaacttcggtaactcttacatttcctactgggcgtattggggccagggcacgcttgtgacggttagttctagcggaggtggtaaaaagctcgctgacgagccagagggaggatcccagactgtggtaacacaggaaccatctttgacagtaagtcctggaggtacggtcacgctcacttgtgggtcctcaaccggggctgtaacgtcaggccattaccctaactgggtccaacagaagcctggacaagctcccaggggtctgataggcggaacttcaaacaagcactcttggactccagcgcgctttagcggttcccttctgggtggaaaagcagccctcactctgagtggagtacaacccgaggatgaggcggaatattattgcgtgctctggggttcacgccgctgggtcttcggtggcggtacgaaacttactgtactggggggaggcggctcaggcggcggatcagaagtgcagcttgttgaatctggcggaggtctggtccagccaggtaacagcttgagactgtcctgtgctgcaagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccactga

SEQ ID NO: 15 - protein, 512 aa, Artificial Sequence
  Synthetic construct - exemplary anti-EGFR hemi-COBRA (Pro42 - alpha-EGFR-2 sdAb - alpha -CD3Vi - Meprin - alpha-CD3VhiGL4 - alpha-HSA - His6)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGGKKLADEPEGGSEVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 16 - DNA, 1539 bp, Artificial Sequence
  Synthetic construct - exemplary anti-EGFR hemi-COBRA (Pro42 - alpha-EGFR-2 sdAb - alpha -CD3Vi - Meprin - alpha-CD3VhiGL4 - alpha-HSA - His6)
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccagacagtcgtaacgcaggagccgtcacttactgtgtctccgggagggacggttactttgacctgtgcgtcttcaaccggcgccgttacctcagggaattaccctaattgggtccaacaaaaaccgggtcaggcacccaggggcctgatcgggggcacaaaatttctcgtcccgggcactccggccaggttttccggttctctgctcgggggtaaggcggccctgacccttagtggtgttcaaccggaggatgaagcagagtattactgcacattgtggtactcaaatcggtgggtttttgggggcggtacaaagctgacagtgttgtcaggagggggcaaaaaactggcggatgaaccagaaggggggtccgaggtccagctcgttgaatctgggggcggcctggtacagccaggaggttccctcaagctttcatgcgccgcatccggattcaccttcagtgggtacgcaatgaattgggttagacaggcaccaggtaaagggttggaatgggtggcacgcattaggtccaaggccaacagctacgccaccgagtacgcggccagcgtaaaagaccgatttacgataagccgggatgattctaagaacactgcttatttgcagatgaataatttgaagaccgaggatactgctgtctattattgcgtccgccacggtaatgctggtaactctgccattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcactga

SEQ ID NO: 17 - protein, 515 aa, Artificial Sequence
  Synthetic construct - exemplary anti-EGFR hemi-COBRA (Pro43 - alpha-EGFR-1 sdAb - alpha- CD3 Vh - Matriptase - alpha-CD3VliGL - alpha-HSA - His6)
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGSFTRQARVVGGGSQTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 18 - DNA, 1548 bp, Artificial Sequence
  Synthetic construct - exemplary anti-EGFR hemi-COBRA (Pro43 - alpha-EGFR-1 sdAb - alpha- CD3 Vh - Matriptase - alpha-CD3VliGL - alpha-HSA - His6)
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatctctcgagatgactccaagaatacagcttaccttcaaatgaataatctgaagacagaggataccgccgtgtattactgcgtccgacatgcgaactttggtaattcttatatctcttattgggcctattggggtcagggcacgttggttaccgtttcttctggaggttcattcacccgccaggcgcgagttgtcggtggaggatcccagactgtggtaacacaggaaccatctttgacagtaagtcctggaggtacggtcacgctcacttgtgggtcctcaaccggggctgtaacgtcaggccattaccctaactgggtccaacagaagcctggacaagctcccaggggtctgataggcggaacttcaaacaagcactcttggactccagcgcgctttagcggttcccttctgggtggaaaagcagccctcactctgagtggagtacaacccgaggatgaggcggaatattattgcgtgctctggggttcacgccgctgggtcttcggtggcggtacgaaacttactgtactggggggaggcggctcaggcggcggatcagaagtgcagcttgttgaatctggcggaggtctggtccagccaggtaacagcttgagactgtcctgtgctgcaagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccactga

SEQ ID NO: 19 - protein, 512 aa, Artificial Sequence
  Synthetic construct - an exemplary anti-EGFR hemi-COBRA (Pro44 - alpha-EGFR-2 sdAb - alpha -CD3Vl - Matriptase - alpha-CD3VhiGL4 - alpha-HSA - His6)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGSFTRQARVVGGGSEVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 20 - DNA, 1539 bp, Artificial Sequence
  Synthetic construct - an exemplary anti-EGFR hemi-COBRA (Pro44 - alpha-EGFR-2 sdAb - alpha -CD3Vl - Matriptase - alpha-CD3VhiGL4 - alpha-HSA - His6)
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtgtttggtggaggcacgaaactgacggtattggggggatcatttacgcgccaagctagagtcgtgggaggtggatcagaggtccagttggtcgagagcggggggggtctggtccaaccagggggtagtctcaagctttcatgcgccgcatccggattcaccttcagtgggtacgcaatgaattgggttagacaggcaccaggtaaagggttggaatgggtggcacgcattaggtccaaggccaacagctacgccaccgagtacgcggccagcgtaaaagaccgatttacgataagccgggatgattctaagaacactgcttatttgcagatgaataatttgaagaccgaggatactgctgtctattattgcgtccgccacggtaatgctggtaactctgccattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcactga

SEQ ID NO: 21 - protein, 515 aa, Artificial Sequence
  Synthetic construct - exemplary anti-EGFR hemi-COBRA (Pro45 - alpha-EGFR-1 sdAb - alpha- CD3 Vh - Thrombin - alpha-CD3VliGL - alpha-HSA - His6)
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSSGGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSSGGGMPRSFRGGGSQTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 22 - DNA, 1548 bp, Artificial Sequence
  Synthetic construct - exemplary anti-EGFR hemi-COBRA (Pro45 - alpha-EGFR-1 sdAb - alpha- CD3 Vh - Thrombin - alpha-CD3VliGL - alpha-HSA - His6)
gaagttcaactggttgaatccggtggtggccttgtccaggcgggaggctcccttcgactgtcttgtgccgccagcggtcgcacattcagcagttatgccatgggatggttccggcaggcccctggtaaagagcgggagttcgtcgttgcgatcaattggagtagcggttccacgtattatgcggattctgtaaagggcaggttcactatctcacgcgataatgcaaaaaataccatgtatcttcagatgaactcactgaagcccgaggacacggcagtttattactgtgctgccggttaccagatcaattccggaaattacaatttcaaggactacgagtacgattattggggtcagggcacccaggtaaccgtcagcagcgggggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatctctcgagatgactcaaagaatacagcatatctgcaaatgaacaatttgaaaacagaagacacggcagtttattactgcgttaggcacgctaacttcggtaattcatacatatcatattgggcctactggggccaagggactttggtcacagtatcctccagctcagggggtggtatgcctcgctctttcagggggggcggatcccagactgtggtaacacaggaaccatctttgacagtaagtcctggaggtacggtcacgctcacttgtgggtcctcaaccggggctgtaacgtcaggccattaccctaactgggtccaacagaagcctggacaagctcccaggggtctgataggcggaacttcaaacaagcactcttggactccagcgcgctttagcggttcccttctgggtggaaaagcagccctcactctgagtggagtacaacccgaggatgaggcggaatattattgcgtgctctggggttcacgccgctgggtcttcggtggcggtacgaaacttactgtactggggggaggcggctcaggcggcggatcagaagtgcagcttgttgaatctggcggaggtctggtccagccaggtaacagcttgagactgtcctgtgctgcaagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagtcatcatcatcatcaccactga

SEQ ID NO: 23 - protein, 512 aa, Artificial Sequence
  Synthetic construct - exemplary anti-EGFR hemi-COBRA (Pro46 - alpha-EGFR-2 sdAb - alpha -CD3Vl - Thrombin - alpha-CD3VhiGL4 - alpha-HSA - His6)
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSSGGGMPRSFRGGGSEVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 24 - DNA, 1539 bp, Artificial Sequence
  Synthetic construct - exemplary anti-EGFR hemi- COBRA (Pro46 - alpha-EGFR-2 sdAb - alpha-CD3Vl - Thrombin - alpha- CD3VhiGL4 - alpha-HSA - His6)
caagtcaaacttgaggaaagcgggggaggtagcgtacagactggtggatctctgaggttgacttgcgccgccagtggccgaacatccagaagttacgggatgggttggtttcgacaggctccgggaaaagagcgggagtttgtatctggcataagctggaggggcgactccactggttacgcagattccgtcaaagggcggtttacgatctctcgggataacgcgaagaataccgttgatctccaaatgaactctcttaaacccgaggatacagcaatatactattgcgccgccgctgcggggtcagcctggtatggcacattgtacgaatatgactattggggtcaaggtacccaagtaacggtcagttccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggagggggtaccaagctgacggtgttgtcatctggcggaggtatgccaaggagctttcgcggtggaggctcagaagtacaacttgtagaaagcggggggggtctggtccagccaggcggaagcctcaagctttcatgcgccgcatccggattcaccttcagtgggtacgcaatgaattgggttagacaggcaccaggtaaagggttggaatgggtggcacgcattaggtccaaggccaacagctacgccaccgagtacgcggccagcgtaaaagaccgatttacgataagccgggatgattctaagaacactgcttatttgcagatgaataatttgaagaccgaggatactgctgtctattattgcgtccgccacggtaatgctggtaactctgccattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcactga

SEQ ID NO: 25 - protein, 536 aa, Artificial Sequence
  Synthetic construct - exemplary anti-EGFR hemi- COBRA (Pro349 - h-alpha-EGFR-2 sdAb - alpha-CD3 Vh - MMP9 - alpha- CD3Vli2 - alpha-HSA - Strep2)
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSSGGPGPAGMKGLPGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSAWSHPQFEKGGGSGGGSGGSSAWSHPQFEK

SEQ ID NO: 26 - DNA, 1611 bp, Artificial Sequence
  Synthetic construct - exemplary anti-EGFR hemi- COBRA (Pro349 - h-alpha-EGFR-2 sdAb - alpha-CD3 Vh - MMP9 - alpha- CD3Vli2 - alpha-HSA - Strep2)
caagttaaactcgtagagtcaggaggtggtgtagtccgtccgggaggtagcttaacccttagctgcgctgctagcggacggacaagccgcagttacggcatggggtggttcagacaagcgccaggcaaggaacgcgagtttgtttctggcatcagttggcgcggggattctacgggctatgcagactccgtgaaggggagattcacgatctctagagacaacgccaagaacagtttgtatctccaaatgaactcacttagggcagaagatactgcactttactactgcgcggctgcagcgggaagtgcatggtacggtacgctttacgagtatgattactggggacaagggaccctcgtcacagttagcagtggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatcagtagagatgacagtaagaacacggcttatttgcaaatgaacaacttgaagacagaagatacggcggtctattattgtgtacgacacgcaaattttgggaattcatatataagctattgggcatactggggtcaaggaacccttgttacggtgagcagctctggggggccagggccagcgggcatgaaaggccttccgggatcccagactgtggtaacacaggaaccatctttgacagtaagtcctggaggtacggtcacgctcacttgtgggtcctcaaccggggctgtaacgtcaggcaattaccctaactgggtccaacagaagcctggacaagctcccaggggtctgataggcggaactaaagatgatgcccctggcactccagcgcgctttagcggatcccttctgggtggaaaagcagccctcactctgagtggagtacaacccgaggatgaggcggaatattattgcgtgctctggtattcaaaccgctgggtcttcggtggcggtacgaaacttactgtactggggggaggcggctcaggcggcggatcagaagtgcagcttgttgaatctggcggaggtctggtccagccaggtaacagcttgagactgtcctgtgctgctagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagttctgcttggtctcatccgcagtttgaaaagggtggggggtctggtggaggaagtggcggttcttctgcctggtcacacccacaattcgagaaatga

SEQ ID NO: 27 - protein, 513 aa, Artificial Sequence
  Synthetic construct - exemplary anti-EGFR hemi- COBRA (Pro353 - h-alpha-EGFR-2 sdAb - alpha-CD3Vl - MMP9 - alpha- CD3Vhi - alpha-HSA - His6)
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLSGGPGPAGMKGLPGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 28 - DNA, 1542 bp, Artificial Sequence
  Synthetic construct - exemplary anti-EGFR hemi- COBRA (Pro353 - h-alpha-EGFR-2 sdAb - alpha-CD3Vl - MMP9 - alpha- CD3Vhi - alpha-HSA - His6)
caagttaaactcgtagagtcaggaggtggtgtagtccgtccgggaggtagcttaacccttagctgcgctgctagcggacggacaagccgcagttacggcatggggtggttcagacaagcgccaggcaaggaacgcgagtttgtttctggcatcagttggcgcggggattctacgggctatgcagactccgtgaaggggagattcacgatctctagagacaacgccaagaacagtttgtatctccaaatgaactcacttagggcagaagatactgcactttactactgcgcggctgcagcgggaagtgcatggtacggtacgctttacgagtatgattactggggacaagggaccctcgtcacagttagcagtggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtactttctggggggccagggccagcgggcatgaaaggccttccgggatccgaggtccagttggtagaatccggaggtggattggttcaaccgggaggaagccttaagctttcatgcgccgcatccggattcaccttcaataagtacgcaatgaattgggttagacaggcaccaggtaaagggttggaatgggtggcacgcattaggtctaaatacgattacaaggacgacgacgacaaagctgacagcgtaaaagaccgatttacgataagccgggatgattctaagaacactgcttatttgcagatgaataatttgaagaccgaggatactgctgtctattattgcgtccgccacggtaattttggtaactcttacattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcactga

SEQ ID NO: 29 - protein, 534 aa, Artificial Sequence
  Synthetic construct - exemplary anti- B7H3 hemi-COBRA (Pro348 - h-alpha-B7H3 sdAb - alpha-CD3Vh - NCL15 - alpha-CD3Vli2 - alpha-HSA - Strep2)
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSAWSHPQFEKGGGSGGGSGGSSAWSHPQFEK

SEQ ID NO: 30 - DNA, 1365 bp, Artificial Sequence
  Synthetic construct - exemplary anti- B7H3 hemi-COBRA (Pro348 - h-alpha-B7H3 sdAb - alpha-CD3Vh - NCL15 - alpha-CD3Vli2 - alpha-HSA - Strep2)
ctgcaaatgaactctttgagagctgaagatacagctgtgtattattgcgctgcgggaggtgtactggcgcatcataactatgagtacgattactggggacagggcacgctcgtaactgtttcatccggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatcagtagagatgacagtaagaacacggcttatttgcaaatgaacaacttgaagacagaagatacggcggtctattattgtgtacgacacgcaaattttgggaattcatatataagctattgggcatactggggtcaaggaacccttgttacggtgagcagcgggggcggtggttctggcgggggaggttccgggggcggcggatcccagactgtggtaacacaggaaccatctttgacagtaagtcctggaggtacggtcacgctcacttgtgggtcctcaaccggggctgtaacgtcaggcaattaccctaactgggtccaacagaagcctggacaagctcccaggggtctgataggcggaactaaagatgatgcccctggcactccagcgcgctttagcggatcccttctgggtggaaaagcagccctcactctgagtggagtacaacccgaggatgaggcggaatattattgcgtgctctggtattcaaaccgctgggtcttcggtggcggtacgaaacttactgtactggggggaggcggctcaggcggcggatcagaagtgcagcttgttgaatctggcggaggtctggtccagccaggtaacagcttgagactgtcctgtgctgctagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagttctgcttggtctcatccgcagtttgaaaagggtggggggtctggtggaggaagtggcggttcttctgcctggtcacacccacaattcgagaaatga

SEQ ID NO: 31 - protein, 511 aa, Artificial Sequence
  Synthetic construct - exemplary anti-B7H3 hemi- COBRA (Pro352 - h-alpha-B7H3 sdAb - alpha-CD3Vl - NCL15 - alpha- CD3Vhi - alpha-HSA - HIs6)
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 32 - DNA, 1533 bp, Artificial Sequence
  Synthetic construct - exemplary anti-B7H3 hemi- COBRA (Pro352 - h-alpha-B7H3 sdAb - alpha-CD3Vl - NCL15 - alpha- CD3Vhi - alpha-HSA - HIs6)
caagtccaactccaggaatccggtggcggtcttgtccaaccagggggttcactccgattgtcctgtgaggccagtccccggacctttagcacttactcaatggcttggttccgccaagcgcctgggaaggaacgctctttcgtagctgctattaactggagcggaggtaacacttcttacgctgactccgtgaaggggcgctttaccataagtagagacaatagcaaaaatacactctacctgcaaatgaactctttgagagctgaagatacagctgtgtattattgcgctgcgggaggtgtactggcgcatcataactatgagtacgattactggggacagggcacgctcgtaactgtttcatccggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttgggggcggtggttctggcgggggaggttccgggggcggcggatccgaggtccagttggtagaatccggaggtggattggttcaaccgggaggaagccttaagctttcatgcgccgcatccggattcaccttcaataagtacgcaatgaattgggttagacaggcaccaggtaaagggttggaatgggtggcacgcattaggtctaaatacgattacaaggacgacgacgacaaagctgacagcgtaaaagaccgatttacgataagccgggatgattctaagaacactgcttatttgcagatgaataatttgaagaccgaggatactgctgtctattattgcgtccgccacggtaattttggtaactcttacattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcac

SEQ ID NO: 33 - protein, 536 aa, Artificial Sequence
  Synthetic construct - exemplary anti-EGFR hemi- COBRA (Pro350 - h-alpha-EGFR-2 sdAb - alpha-CD3Vh - NCL15 - alpha- CD3Vli2 - alpha-HSA - Strep2)
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGSGGGSEVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSSGGGGSGGGGSGGGGSQTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVLGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSSAWSHPQFEKGGGSGGGSGGSSAWSHPQFEK

SEQ ID NO: 34 - DNA, 1611 bp, Artificial Sequence
  Synthetic construct - exemplary anti-EGFR hemi- COBRA (Pro350 - h-alpha-EGFR-2 sdAb - alpha-CD3Vh - NCL15 - alpha- CD3Vli2 - alpha-HSA - Strep2)
caagttaaactcgtagagtcaggaggtggtgtagtccgtccgggaggtagcttaacccttagctgcgctgctagcggacggacaagccgcagttacggcatggggtggttcagacaagcgccaggcaaggaacgcgagtttgtttctggcatcagttggcgcggggattctacgggctatgcagactccgtgaaggggagattcacgatctctagagacaacgccaagaacagtttgtatctccaaatgaactcacttagggcagaagatactgcactttactactgcgcggctgcagcgggaagtgcatggtacggtacgctttacgagtatgattactggggacaagggaccctcgtcacagttagcagtggaggcggatcaggaggcggttcagaggttcagctcgttgagagtggtggagggctggttcagccagggggaagtttgaagctttcctgtgcggcctctggtttcacctttaacaaatacgctatcaactgggtacgacaagcccccggtaaagggcttgaatgggttgcaagaatacgcagtaaatacaataattatgcgacttattatgccgatcaagtaaaggaccgctttactatcagtagagatgacagtaagaacacggcttatttgcaaatgaacaacttgaagacagaagatacggcggtctattattgtgtacgacacgcaaattttgggaattcatatataagctattgggcatactggggtcaaggaacccttgttacggtgagcagcgggggcggtggttctggcgggggaggttccgggggcggcggatcccagactgtggtaacacaggaaccatctttgacagtaagtcctggaggtacggtcacgctcacttgtgggtcctcaaccggggctgtaacgtcaggcaattaccctaactgggtccaacagaagcctggacaagctcccaggggtctgataggcggaactaaagatgatgcccctggcactccagcgcgctttagcggatcccttctgggtggaaaagcagccctcactctgagtggagtacaacccgaggatgaggcggaatattattgcgtgctctggtattcaaaccgctgggtcttcggtggcggtacgaaacttactgtactggggggaggcggctcaggcggcggatcagaagtgcagcttgttgaatctggcggaggtctggtccagccaggtaacagcttgagactgtcctgtgctgctagcggctttaccttctctaaattcggtatgagttgggttcggcaagcccctggaaagggtttggaatgggtatcaagcattagtggttctgggcgagatacactctatgccgaatcagtgaagggccgctttaccattagtagggataacgctaaaactactctgtatctgcaaatgaatagtctgagaccagaagatactgccgtttactactgcacaatagggggatctctgagcgtttcatctcaaggtacacttgtgactgttagcagttctgcttggtctcatccgcagtttgaaaagggtggggggtctggtggaggaagtggcggttcttctgcctggtcacacccacaattcgagaaatga

SEQ ID NO: 35 - protein, 513 aa, Artificial Sequence
  Synthetic construct - exemplary anti-EGFR hemi- COBRA (Pro354 - h-alpha-EGFR-2 sdAb - alpha-CD3Vl - NCL15 - alpha- CD3Vhi - alpha-HSA - His6)
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSSGGGGSGGGSQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVLGGGGSGGGGSGGGGSEVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSSGGGGSGGGSEVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 36 - DNA, 1542 bp, Artificial Sequence
  Synthetic construct - exemplary anti-EGFR hemi- COBRA (Pro354 - h-alpha-EGFR-2 sdAb - alpha-CD3Vl - NCL15 - alpha- CD3Vhi - alpha-HSA - His6)
caagttaaactcgtagagtcaggaggtggtgtagtccgtccgggaggtagcttaacccttagctgcgctgctagcggacggacaagccgcagttacggcatggggtggttcagacaagcgccaggcaaggaacgcgagtttgtttctggcatcagttggcgcggggattctacgggctatgcagactccgtgaaggggagattcacgatctctagagacaacgccaagaacagtttgtatctccaaatgaactcacttagggcagaagatactgcactttactactgcgcggctgcagcgggaagtgcatggtacggtacgctttacgagtatgattactggggacaagggaccctcgtcacagttagcagtggtggtggggggtctggtggtggatcccaaacagttgttactcaggagccatccttgacagtatcccccggtggaacggtgaccctgacatgcgcttcaagtacaggtgctgtaacctcaggtaactacccgaattgggtgcagcaaaaacctggacaagcaccccggggtcttatcggggggacgaagttcttggtaccgggtacccctgcgcgcttcagcggaagtcttctgggtggaaaagccgccttgaccttgtcaggcgttcagcccgaagatgaggccgaatattattgcacgctgtggtattctaaccggtgggtcttcggaggagggacgaaacttactgtacttgggggcggtggttctggcgggggaggttccgggggcggcggatccgaggtccagttggtagaatccggaggtggattggttcaaccgggaggaagccttaagctttcatgcgccgcatccggattcaccttcaataagtacgcaatgaattgggttagacaggcaccaggtaaagggttggaatgggtggcacgcattaggtctaaatacgattacaaggacgacgacgacaaagctgacagcgtaaaagaccgatttacgataagccgggatgattctaagaacactgcttatttgcagatgaataatttgaagaccgaggatactgctgtctattattgcgtccgccacggtaattttggtaactcttacattagctattgggcgtattgggggcagggcactctggtcaccgtctcatctggcggagggggcagtggcggcgggtcagaggttcaacttgtcgagtctggaggcggtctcgtacaaccggggaatagtctccgactctcttgcgctgcgtccgggttcacgttctcaaagtttgggatgtcttgggttaggcaagccccaggtaagggactcgaatgggtcagcagcatctcaggctccggcagagacacgttgtatgccgaaagtgtcaaagggaggttcacaatctctcgggacaatgcaaaaaccaccttgtatctccaaatgaactcactccggcctgaggacacagcagtttactactgtacgataggagggtcccttagcgtatcttctcagggaactttggtaacggtcagctcccaccaccatcatcatcactga

SEQ ID NO: 37 - protein, 127 aa, Artificial Sequence
  Synthetic construct - exemplary targeting sdAbs - alpha-EGFR1
EVQLVESGGGLVQAGGSLRLSCAASGRTFSSYAMGWFRQAPGKEREFVVAINWSSGSTYYADSVKGRFTISRDNAKNTMYLQMNSLKPEDTAVYYCAAGYQINSGNYNFKDYEYDYWGQGTQVTVSS

SEQ ID NO: 38 - protein, 10 aa, Artificial Sequence
  Synthetic construct - exemplary targeting sdAbs - sdCDR1 of alpha-EGFR1
GRTFSSYAMG

SEQ ID NO: 39 - protein, 16 aa, Artificial Sequence
  Synthetic construct - exemplary targeting sdAbs - sdCDR2 of alpha-EGFR1
INWSSGSTYYADSVKG

SEQ ID NO: 40 - protein, 18 aa, Artificial Sequence
  Synthetic construct - exemplary targeting sdAbs - sdCDR3 of alpha-EGFR1
GYQINSGNYNFKDYEYDY

SEQ ID NO: 41 - protein, 124 aa, Artificial Sequence
  Synthetic construct - exemplary targeting sdAbs - alpha-EGFR2
QVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 42 - protein, 10 aa, Artificial Sequence
  Synthetic construct - exemplary targeting sdAbs - sdCDR1 of alpha-EGFR2
GRTSRSYGMG

SEQ ID NO: 43 - protein, 17 aa, Artificial Sequence
  Synthetic construct - exemplary targeting sdAbs - sdCDR2 of alpha-EGFR2
GISWRGDSTGYADSVKG

SEQ ID NO: 44 - protein, 15 aa, Artificial Sequence
  Synthetic construct - exemplary targeting sdAbs - sdCDR3 of alpha-EGFR2
AAGSAWYGTLYEYDY

SEQ ID NO: 45 - protein, 127 aa, Artificial Sequence
  Synthetic construct - exemplary targeting sdAbs - h-alpha-EGFR1
EVQLVESGGGLVQPGGSLRLSCAASGRTFSSYAMGWFRQAPGKEREFVVAINWSSGSTYYADSVKGRFTISRDNSKNTLYLQMNSLRAEDTAVYYCAAGYQINSGNYNFKDYEYDYWGQGTLVTVSS

SEQ ID NO: 46 - protein, 10 aa, Artificial Sequence
  Synthetic construct - exemplary targeting sdAbs - sdCDR1 of h-alpha-EGFR1
GRTFSSYAMG

SEQ ID NO: 47 - protein, 16 aa, Artificial Sequence
  Synthetic construct - exemplary targeting sdAbs - sdCDR2 of h-alpha-EGFR1
INWSSGSTYYADSVKG

SEQ ID NO: 48 - protein, 18 aa, Artificial Sequence
  Synthetic construct - exemplary targeting sdAbs - sdCDR3 of h-alpha-EGFR1
GYQINSGNYNFKDYEYDY

SEQ ID NO: 49 - skipped

SEQ ID NO: 50 - protein, 124 aa, Artificial Sequence
  Synthetic construct - exemplary targeting sdAbs - h-alpha-EGFR2
QVKLVESGGGVVRPGGSLTLSCAASGRTSRSYGMGWFRQAPGKEREFVSGISWRGDSTGYADSVKGRFTISRDNAKNSLYLQMNSLRAEDTALYYCAAAAGSAWYGTLYEYDYWGQGTLVTVSS

SEQ ID NO: 51 - protein, 10 aa, Artificial Sequence
  Synthetic construct - exemplary targeting sdAbs - sdCDR1 of h-alpha-EGFR2
GRTSRSYGMG

SEQ ID NO: 52 - protein, 17 aa, Artificial Sequence
  Synthetic construct - exemplary targeting sdAbs - sdCDR2 of h-alpha-EGFR2
GISWRGDSTGYADSVKG

SEQ ID NO: 53 - protein, 15 aa, Artificial Sequence
  Synthetic construct - exemplary targeting sdAbs - sdCDR3 of h-alpha-EGFR2
AAGSAWYGTLYEYDY

SEQ ID NO: 54 - protein, 114 aa, Artificial Sequence
  Synthetic construct - exemplary targeting sdAbs - alpha-FOLR1 h77-2
QVQLVESGGGLVQPGGSLRLSCAASGFTVSNSVMAWYRQTPGNEREFVAIINSIGITNYADSVKGRFTISRDNSKNTLYLQMNSLRAEDTAVYVCNRNFDRIYWGQGTLVTVSS

SEQ ID NO: 55 - protein, 10 aa, Artificial Sequence
  Synthetic construct - exemplary targeting sdAbs - sdCDR1 of alpha-FOLR1 h77-2
GFTVSNSVMA

SEQ ID NO: 56 - protein, 16 aa, Artificial Sequence
  Synthetic construct - exemplary targeting sdAbs - sdCDR2 of alpha-FOLR1 h77-2
IINSIGITNYADSVKG

SEQ ID NO: 57 - protein, 6 aa, Artificial Sequence
  Synthetic construct - exemplary targeting sdAbs - sdCDR3 of alpha-FOLR1 h77-2
NFDRIY

SEQ ID NO: 58 - protein, 113 aa, Artificial Sequence
  Synthetic construct - exemplary targeting sdAbs - alpha-FOLR1 h59.3
QVQLVESGGGLVQPGGSLRLSCAAPGNTFSISAMGWYRQAPGKQREWVAVTHSDYSTNYADSVKGRFTISRDNSKNTLYLQMNSLRAEDTAVYYCKHYGIDYWGQGTLVTVSS

SEQ ID NO: 59 - protein, 10 aa, Artificial Sequence
  Synthetic construct - exemplary targeting sdAbs - sdCDR1 of alpha-FOLR1 h59.3
GNTFSISAMG

SEQ ID NO: 60 - protein, 16 aa, Artificial Sequence
  Synthetic construct - exemplary targeting sdAbs - sdCDR2 of alpha-FOLR1 h59.3
VTHSDYSTNYADSVKG

SEQ ID NO: 61 - protein, 5 aa, Artificial Sequence
  Synthetic construct - exemplary targeting sdAbs - sdCDR3 of alpha-FOLR1 h59.3
YGIDY

SEQ ID NO: 62 - protein, 117 aa, Artificial Sequence
  Synthetic construct - exemplary targeting sdAbs - alpha-FOLR1 h22-4
QVQLVESGGGLVQPGGSLRLSCEASGTTFSRDVMGWYRQAPGKQRELVAIISRGGSTNYADSVKGRFTISRDNSKNTLYLQMNSLRAEDTAVYYCNANTATWGRVFWGQGTLVTVSS

SEQ ID NO: 63 - protein, 10 aa, Artificial Sequence
  Synthetic construct - exemplary targeting sdAbs - sdCDR1 of alpha-FOLR1 h22-4
GTTFSRDVMG

SEQ ID NO: 64 - protein, 16 aa, Artificial Sequence
  Synthetic construct - exemplary targeting sdAbs - sdCDR2 of alpha-FOLR1 h22-4
IISRGGSTNYADSVKG

SEQ ID NO: 65 - protein, 9 aa, Artificial Sequence
  Synthetic construct - exemplary targeting sdAbs - sdCDR3 of alpha-FOLR1 h22-4
NTATWGRVF

SEQ ID NO: 66 - protein, 124 aa, Artificial Sequence
  Synthetic construct - exemplary targeting sdAbs - alpha-B7H3 hF7
QVQLQESGGGLVQPGGSLRLSCAPSRRTFHTYHMGWFRQAPGKEREFVAVINWSGGSTVYADSVKGRFTISRDNSKNTLYLQMNSLRAEDTAVYYCAAGGATTQRATEASYDYWGQGTLVTVSS

SEQ ID NO: 67 - protein, 10 aa, Artificial Sequence
  Synthetic construct - exemplary targeting sdAbs - sdCDR1 of alpha-B7H3 hF7
RRTFHTYHMG

SEQ ID NO: 68 - protein, 17 aa, Artificial Sequence
  Synthetic construct - exemplary targeting sdAbs - sdCDR2 of alpha-B7H3 hF7
VINWSGGSTVYADSVKG

SEQ ID NO: 69 - protein, 15 aa, Artificial Sequence
  Synthetic construct - exemplary targeting sdAbs - sdCDR3 of alpha-B7H3 hF7
GGATTQRATEASYDY

SEQ ID NO: 70 - protein, 122 aa, Artificial Sequence
  Synthetic construct - exemplary targeting sdAbs - alpha-B7H3 hF12
QVQLQESGGGLVQPGGSLRLSCEASPRTFSTYSMAWFRQAPGKERSFVAAINWSGGNTSYADSVKGRFTISRDNSKNTLYLQMNSLRAEDTAVYYCAAGGVLAHHNYEYDYWGQGTLVTVSS

SEQ ID NO: 71 - protein, 10 aa, Artificial Sequence
  Synthetic construct - exemplary targeting sdAbs - sdCDR1 of alpha-B7H3 hF12
PRTFSTYSMA

SEQ ID NO: 72 - protein, 17 aa, Artificial Sequence
  Synthetic construct - exemplary targeting sdAbs - sdCDR2 of alpha-B7H3 hF12
AINWSGGNTSYADSVKG

SEQ ID NO: 73 - protein, 13 aa, Artificial Sequence
  Synthetic construct - exemplary targeting sdAbs - sdCDR3 of alpha-B7H3 hF12
GGVLAHHNYEYDY

SEQ ID NO: 74 - protein, 122 aa, Artificial Sequence
  Synthetic construct - exemplary targeting sdAbs - alpha-EpCAM hF13
QVQLVESGGGLVQPGGSLTLSCAASGTGSIFSINLMGWYRQAPGKQRELVARITSGDSTVYADSVKGRFTISRDNSKNTLYLQMNSLRPEDTAVYYCNLLLRSSPGATTPYWGQGTLVTVSS

SEQ ID NO: 75 - protein, 12 aa, Artificial Sequence
  Synthetic construct - exemplary targeting sdAbs - sdCDR1 of alpha-EpCAM hF13
GTGSIFSINLMG

SEQ ID NO: 76 - protein, 16 aa, Artificial Sequence
  Synthetic construct - exemplary targeting sdAbs - sdCDR2 of alpha-EpCAM hF13
RITSGDSTVYADSVKG

SEQ ID NO: 77 - protein, 12 aa, Artificial Sequence
  Synthetic construct - exemplary targeting sdAbs - sdCDR3 of alpha-EpCAM hF13
LLRSSPGATTPY

SEQ ID NO: 78 - protein, 114 aa, Artificial Sequence
  Synthetic construct - exemplary targeting sdAbs alpha-EpCAM hF23
QVQLVESGGGLVQPGGSLTLSCVISGSFSALWAMRWYRQAPGQQRELVASSRGGTTSYADSVKGRFTISRDNSKNTLYLQMNSLRPEDTAVYYCNAIDGHLAYWGQGTLVTVSS

SEQ ID NO: 79 - protein, 10 aa, Artificial Sequence
  Synthetic construct - exemplary targeting sdAbs sdCDR1 of alpha-EpCAM hF23
GSFSALWAMR

SEQ ID NO: 80 - protein, 15 aa, Artificial Sequence
  Synthetic construct - exemplary targeting sdAbs sdCDR2 of alpha-EpCAM hF23
SSRGGTTSYADSVKG

SEQ ID NO: 81 - protein, 7 aa, Artificial Sequence
  Synthetic construct - exemplary targeting sdAbs sdCDR3 of alpha-EpCAM hF23
IDGHLAY

SEQ ID NO: 82 - protein, 115 aa, Artificial Sequence
  Synthetic construct - exemplary alpha-HSA half-life extension domain
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSS

SEQ ID NO: 83 - protein, 10 aa, Artificial Sequence
  Synthetic construct - exemplary sdCDR1 of alpha-HSA half-life extension domain
GFTFSKFGMS

SEQ ID NO: 84 - protein, 17 aa, Artificial Sequence
  Synthetic construct - exemplary sdCDR2 of alpha-HSA half-life extension domain
SISGSGRDTLYAESVKG

SEQ ID NO: 85 - protein, 6 aa, Artificial Sequence
  Synthetic construct - exemplary sdCDR3 of alpha-HSA half-life extension domain
GGSLSV

SEQ ID NO: 86 - protein, 121 aa, Artificial Sequence
  Synthetic construct - exemplary alpha-HSA half-life extension domain with His tag
EVQLVESGGGLVQPGNSLRLSCAASGFTFSKFGMSWVRQAPGKGLEWVSSISGSGRDTLYAESVKGRFTISRDNAKTTLYLQMNSLRPEDTAVYYCTIGGSLSVSSQGTLVTVSSHHHHHH

SEQ ID NO: 87 - protein, 10 aa, Artificial Sequence
  Synthetic construct - exemplary sdCDR1 of alpha-HSA half-life extension domain with His tag
GFTFSKFGMS

SEQ ID NO: 88 - protein, 17 aa, Artificial Sequence
  Synthetic construct - exemplary sdCDR2 of alpha-HSA half-life extension domain with His tag
SISGSGRDTLYAESVKG

SEQ ID NO: 89 - protein, 6 aa, Artificial Sequence
  Synthetic construct - exemplary sdCDR3 of alpha-HSA half-life extension domain with His tag
GGSLSV

SEQ ID NO: 90 - protein, 109 aa, Artificial Sequence
  Synthetic construct - exemplary active anti-CD3 variable light domain (alpha-CD3 scFv domain)
QTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 91 - protein, 14 aa, Artificial Sequence
  Synthetic construct - exemplary aVLCDR1 of active anti-CD3 variable light domain (alpha-CD3 scFv domain)
ASSTGAVTSGNYPN

SEQ ID NO: 92 - protein, 7 aa, Artificial Sequence
  Synthetic construct - aVLsdCDR2 of exemplary active anti-CD3 variable light domain (alpha-CD3 scFv domain)
GTKFLVP

SEQ ID NO: 93 - protein, 9 aa, Artificial Sequence
  Synthetic construct - aVLsdCDR3 of exemplary active anti-CD3 variable light domain (alpha-CD3 scFv domain)
TLWYSNRWV

SEQ ID NO: 94 - protein, 110 aa, Artificial Sequence
  Synthetic construct - exemplary inactive anti-CD3 variable light domain (alpha-CD3 scFv domain)
QTVVTQEPSLTVSPGGTVTLTCGSSTGAVTSGNYPNWVQQKPGQAPRGLIGDYKDDDDKGTPARFSGSLLGGKAALTLSGVQPEDEAEYYCVLWYSNRWVFGGGTKLTVL

SEQ ID NO: 95 - protein, 14 aa, Artificial Sequence
  Synthetic construct - exemplary iVLCDR1 of inactive anti-CD3 variable light domain (alpha-CD3 scFv domain)
GSSTGAVTSGNYPN

SEQ ID NO: 96 - protein, 8 aa, Artificial Sequence
  Synthetic construct - exemplary iVLsdCDR2 of inactive anti-CD3 variable light domain (alpha-CD3 scFv domain)
DYKDDDDK

SEQ ID NO: 97 - protein, 9 aa, Artificial Sequence
  Synthetic construct - exemplary iVLsdCDR3 of inactive anti-CD3 variable light domain (alpha-CD3 scFv domain)
VLWYSNRWV

SEQ ID NO: 98 - protein, 109 aa, Artificial Sequence
  Synthetic construct - exemplary inactive anti-CD3 variable light domain (alpha-CD3 scFv domain)
QTVVTQEPSLTVSPGGTVTLTCGSSTGAVTSGNYPNWVQQKPGQAPRGLIGGTKDDAPGTPARFSGSLLGGKAALTLSGVQPEDEAEYYCVLWYSNRWVFGGGTKLTVL

SEQ ID NO: 99 - protein, 14 aa, Artificial Sequence
  Synthetic construct - exemplary iVLCDR1 of inactive anti-CD3 variable light domain (alpha-CD3 scFv domain)
GSSTGAVTSGNYPN

SEQ ID NO: 100 - protein, 7 aa, Artificial Sequence
  Synthetic construct - exemplary iVLsdCDR2 of inactive anti-CD3 variable light domain (alpha-CD3 scFv domain)
GTKDDAP

SEQ ID NO: 101 - protein, 9 aa, Artificial Sequence
  Synthetic construct - exemplary iVLsdCDR3 of inactive anti-CD3 variable light domain (alpha-CD3 scFv domain)
VLWYSNRWV

SEQ ID NO: 102 - protein, 125 aa, Artificial Sequence
  Synthetic construct - exemplary active anti-CD3 variable heavy domain (alpha-CD3 scFv domain)
EVQLVESGGGLVQPGGSLKLSCAASGFTFNKYAINWVRQAPGKGLEWVARIRSKYNNYATYYADQVKDRFTISRDDSKNTAYLQMNNLKTEDTAVYYCVRHANFGNSYISYWAYWGQGTLVTVSS

SEQ ID NO: 103 - protein, 10 aa, Artificial Sequence
  Synthetic construct - exemplary aVHCDR1 of active anti-CD3 variable heavy domain (alpha-CD3 scFv domain)
GFTFNKYAIN

SEQ ID NO: 104 - protein, 19 aa, Artificial Sequence
  Synthetic construct - exemplary aVHsdCDR2 of active anti-CD3 variable heavy domain (alpha-CD3 scFv domain)
RIRSKYNNYATYYADQVKD

SEQ ID NO: 105 - protein, 14 aa, Artificial Sequence
  Synthetic construct - exemplary aVHsdCDR3 of active anti-CD3 variable heavy domain (alpha-CD3 scFv domain)
HANFGNSYISYWAY

SEQ ID NO: 106 - protein, 126 aa, Artificial Sequence
  Synthetic construct - exemplary inactive anti-CD3 variable heavy domain (alpha-CD3 scFv domain)
EVQLVESGGGLVQPGGSLKLSCAASGFTFNKYAMNWVRQAPGKGLEWVARIRSKYDYKDDDDKADSVKDRFTISRDDSKNTAYLQMNNLKTEDTAVYYCVRHGNFGNSYISYWAYWGQGTLVTVSS

SEQ ID NO: 107 - protein, 10 aa, Artificial Sequence
  Synthetic construct - exemplary iVHsdCDR1 of inactive anti-CD3 variable heavy domain (alpha-CD3 scFv domain)
GFTFNKYAMN

SEQ ID NO: 108 - protein, 20 aa, Artificial Sequence
  Synthetic construct - exemplary iVHsdCDR2 of inactive anti-CD3 variable heavy domain (alpha-CD3 scFv domain)
RIRSKYDYKDDDDKADSVKD

SEQ ID NO: 109 - protein, 14 aa, Artificial Sequence
  Synthetic construct - exemplary iVHsdCDR3 of inactive anti-CD3 variable heavy domain (alpha-CD3 scFv domain)
HGNFGNSYISYWAY

SEQ ID NO: 110 - protein, 125 aa, Artificial Sequence
  Synthetic construct - exemplary inactive anti-CD3 variable heavy domain (alpha-CD3 scFv domain)
EVQLVESGGGLVQPGGSLKLSCAASGFTFNKHAMNWVRQAPGKGLEWVARIRSKYNNYATAYADSVKDRFTISRDDSKNTAYLQMNNLKTEDTAVYYCVRHGNFGNSYISYWAYWGQGTLVTVSS

SEQ ID NO: 111 - protein, 10 aa, Artificial Sequence
  Synthetic construct - exemplary iVHsdCDR1 of inactive anti-CD3 variable heavy domain (alpha-CD3 scFv domain)
GFTFNKHAMN

SEQ ID NO: 112 - protein, 19 aa, Artificial Sequence
  Synthetic construct - exemplary iVHsdCDR2 of inactive anti-CD3 variable heavy domain (alpha-CD3 scFv domain)
RIRSKYNNYATAYADSVKD

SEQ ID NO: 113 - protein, 14 aa, Artificial Sequence
  Synthetic construct - exemplary iVHsdCDR3 of inactive anti-CD3 variable heavy domain (alpha-CD3 scFv domain)
HGNFGNSYISYWAY

SEQ ID NO: 114 - protein, 15 aa, Artificial Sequence
  Synthetic construct: Normal/Non-cleavable linker
GGGGSGGGGSGGGGS

SEQ ID NO: 115 - protein, 8 aa, Artificial Sequence
  Synthetic construct: Constrained linker
GGGSGGGS

SEQ ID NO: 116 - protein, 8 aa, Artificial Sequence
  Synthetic protease cleavage site - MMP7
KRALGLPG

SEQ ID NO: 117 - protein, 24 aa, Artificial Sequence
  Synthetic protease cleavage site - MMP7
  misc_feature (1)..(8): X can be Asp or Glu
  misc_feature (17)..(24): X can be Asp or Arg
XXXXXXXXRPLALWRSXXXXXXXX

SEQ ID NO: 118 - protein, 8 aa, Artificial Sequence
  Synthetic protease cleavage site - MMP9
PRSTLIST

SEQ ID NO: 119 - protein, 5 aa, Artificial Sequence
  Synthetic protease cleavage site - MMP9
LEATA

SEQ ID NO: 120 - protein, 10 aa, Artificial Sequence
  Synthetic protease cleavage site - MMP11
GGAANLVRGG

SEQ ID NO: 121 - protein, 10 aa, Artificial Sequence
  Synthetic protease cleavage site - MMP14
SGRIGFLRTA

SEQ ID NO: 122 - protein, 6 aa, Artificial Sequence
  Synthetic protease cleavage site - MMP
PLGLAG

SEQ ID NO: 123 - protein, 6 aa, Artificial Sequence
  Synthetic protease cleavage site - MMP
  misc_feature (6)..(6): Xaa can be any naturally occurring amino acid
PLGLAX

SEQ ID NO: 124 - protein, 7 aa, Artificial Sequence
  Synthetic protease cleavage site - MMP
  misc_feature (5)..(5): X can be Met or Glu
PLGCXAG

SEQ ID NO: 125 - protein, 8 aa, Artificial Sequence
  Synthetic protease cleavage site - MMP
ESPAYYTA

SEQ ID NO: 126 - protein, 6 aa, Artificial Sequence
  Synthetic protease cleavage site - MMP
RLQLKL

SEQ ID NO: 127 - protein, 7 aa, Artificial Sequence
  Synthetic protease cleavage site - MMP
RLQLKAC

SEQ ID NO: 128 - protein, 7 aa, Artificial Sequence
  Synthetic protease cleavage site - MMP2, MMP9, MMP14
  misc_feature (3)..(3): X can be Cys or Ile or Thr
  misc_feature (5)..(5): X can be His or Orn or Phe
EPXGXYL

SEQ ID NO: 129 - protein, 5 aa, Artificial Sequence
  Synthetic protease cleavage site - Urokinase plasminogen activator (upa)
SGRSA

SEQ ID NO: 130 - protein, 4 aa, Artificial Sequence
  Synthetic protease cleavage site - Urokinase plasminogen activator (upa)
DAFK

SEQ ID NO: 131 - protein, 5 aa, Artificial Sequence
  Synthetic protease cleavage site - Urokinase plasminogen activator (upa)
GGGRR

SEQ ID NO: 132 - protein, 4 aa, Artificial Sequence
  Synthetic protease cleavage site - Lysosomal enzyme
GFLG

SEQ ID NO: 133 - protein, 4 aa, Artificial Sequence
  Synthetic protease cleavage site - Lysosomal enzyme
ALAL

SEQ ID NO: 134 - protein, 2 aa, Artificial Sequence
  Synthetic protease cleavage site - Lysosomal enzyme
FK

SEQ ID NO: 135 - protein, 3 aa, Artificial Sequence
  Synthetic protease cleavage site - Cathepsin B
NLL

SEQ ID NO: 136 - protein, 6 aa, Artificial Sequence
  Synthetic protease cleavage site - Cathepsin D
  misc_feature (4)..(4): X can be Glu or Thr
PICXFF

SEQ ID NO: 137 - protein, 8 aa, Artificial Sequence
  Synthetic protease cleavage site - Cathepsin K
GGPRGLPG

SEQ ID NO: 138 - protein, 6 aa, Artificial Sequence
  Synthetic protease cleavage site - Prostate specific antigen
HSSKLQ

SEQ ID NO: 139 - protein, 7 aa, Artificial Sequence
  Synthetic protease cleavage site - Prostate specific antigen
HSSKLQL

SEQ ID NO: 140 - protein, 9 aa, Artificial Sequence
  Synthetic protease cleavage site - Prostate specific antigen
HSSKLQEDA

SEQ ID NO: 141 - protein, 10 aa, Artificial Sequence
  Synthetic protease cleavage site - Herpes simplex virus protease
LVLASSSFGY

SEQ ID NO: 142 - protein, 10 aa, Artificial Sequence
  Synthetic protease cleavage site - Hiv protease
GVSQNYPIVG

SEQ ID NO: 143 - protein, 10 aa, Artificial Sequence
  Synthetic protease cleavage site - Cmv protease
GVVQASCRLA

SEQ ID NO: 144 - protein, 4 aa, Artificial Sequence
  Synthetic protease cleavage site - Thrombin
  misc_feature (2)..(2): X can be Pro or Ile or Pro
FXRS

SEQ ID NO: 145 - protein, 6 aa, Artificial Sequence
  Synthetic protease cleavage site - Thrombin
DPRSFL

SEQ ID NO: 146 - protein, 6 aa, Artificial Sequence
  Synthetic protease cleavage site - Thrombin
PPRSFL

SEQ ID NO: 147 - protein, 4 aa, Artificial Sequence
  Synthetic protease cleavage site - Caspase-3
DEVD

SEQ ID NO: 148 - protein, 5 aa, Artificial Sequence
  Synthetic protease cleavage site - Caspase-3
DEVDP

SEQ ID NO: 149 - protein, 8 aa, Artificial Sequence
  Synthetic protease cleavage site - Caspase-3
KGSGDVEG

SEQ ID NO: 150 - protein, 6 aa, Artificial Sequence
  Synthetic protease cleavage site - Interleukin 1-beta converting enzyme
GWEHDG

SEQ ID NO: 151 - protein, 7 aa, Artificial Sequence
  Synthetic protease cleavage site - Enterokinase
EDDDDKA

SEQ ID NO: 152 - protein, 9 aa, Artificial Sequence
  Synthetic protease cleavage site - Fap
KQEQNPGST

SEQ ID NO: 153 - protein, 6 aa, Artificial Sequence
  Synthetic protease cleavage site - Kallikrein 2
GKAFRR

SEQ ID NO: 154 - protein, 4 aa, Artificial Sequence
  Synthetic protease cleavage site - Plasmin
DAFK

SEQ ID NO: 155 - protein, 4 aa, Artificial Sequence
  Synthetic protease cleavage site - Plasmin
DVLK

SEQ ID NO: 156 - protein, 4 aa, Artificial Sequence
  Synthetic protease cleavage site - Plasmin
DAFK

SEQ ID NO: 157 - protein, 7 aa, Artificial Sequence
  Synthetic protease cleavage site - Top
ALLLALL

SEQ ID NO: 158 - protein, 8 aa, Artificial Sequence
  Synthetic protease cleavage site - MMP (CCL)
GPAGMKGL

SEQ ID NO: 159 - protein, 15 aa, Artificial Sequence
  Synthetic protease cleavage site - MMP (NCCL)
SGGPGPAGMKGLPGS

SEQ ID NO: 160 - protein, 15 aa, Artificial Sequence
  Synthetic protease cleavage site - Cathepsin S/L/K (CCL)
SGGGARLQSAAPGGS

SEQ ID NO: 161 - protein, 8 aa, Artificial Sequence
  Synthetic protease cleavage site - Cathepsin S/L/K (CCL)
ARLQSAAP

SEQ ID NO: 162 - protein, 17 aa, Artificial Sequence
  Synthetic protease cleavage site - Meprin/Granzyme B (CCL)
SGGGGVYADSLEDGGGS

SEQ ID NO: 163 - protein, 10 aa, Artificial Sequence
  Synthetic protease cleavage site - Meprin/Granzyme B (CCL)
GVYADSLEDG

SEQ ID NO: 164 - protein, 16 aa, Artificial Sequence
  Synthetic protease cleavage site - Meprin A/B (CCL)
GGGGKKLADEPEGGGS

SEQ ID NO: 165 - protein, 8 aa, Artificial Sequence
  Synthetic protease cleavage site - Meprin A/B (CCL)
KKLADEPE

SEQ ID NO: 166 - protein, 8 aa, Artificial Sequence
  Synthetic protease cleavage site - MMP9/2
GPAGMKGL

SEQ ID NO: 167 - protein, 15 aa, Artificial Sequence
  Synthetic protease cleavage site - MMP9/2
SGGPGPAGMKGLPGS

SEQ ID NO: 168 - protein, 17 aa, Artificial Sequence
  Synthetic protease cleavage site - MMP9/2
SGGGPGPAGMKGLPGGS

SEQ ID NO: 169 - protein, 17 aa, Artificial Sequence
  Synthetic protease cleavage site - Matriptase (ST14)
SGGGSFTRQARVVGGGS

SEQ ID NO: 170 - protein, 9 aa, Artificial Sequence
  Synthetic protease cleavage site - Matriptase (ST14)
SFTRQARVV

SEQ ID NO: 171 - protein, 15 aa, Artificial Sequence
  Synthetic positively charged scFv linker - Gly-Ser 15
GGGGSGGGGSGGGGS

SEQ ID NO: 172 - protein, 18 aa, Artificial Sequence
  Synthetic positively charged scFv linker - Whitlow linker
GSTSGSGKPGSGEGSTKG

SEQ ID NO: 173 - protein, 14 aa, Artificial Sequence
  Synthetic positively charged scFv linker - 6paxA_1 (+A)
IRPRAIGGSKPRVA

SEQ ID NO: 174 - protein, 15 aa, Artificial Sequence
  Synthetic positively charged scFv linker - (+B)
GKGGSGKGGSGKGGS

SEQ ID NO: 175 - protein, 15 aa, Artificial Sequence
  Synthetic positively charged scFv linker - (+C)
GGKGSGGKGSGGKGS

SEQ ID NO: 176 - protein, 15 aa, Artificial Sequence
  Synthetic positively charged scFv linker - (+D)
GGGKSGGGKSGGGKS

SEQ ID NO: 177 - protein, 15 aa, Artificial Sequence
  Synthetic positively charged scFv linker - (+E)
GKGKSGKGKSGKGKS

SEQ ID NO: 178 - protein, 15 aa, Artificial Sequence
  Synthetic positively charged scFv linker - (+F)
GGGKSGGKGSGKGGS

SEQ ID NO: 179 - protein, 15 aa, Artificial Sequence
  Synthetic positively charged scFv linker - (+G)
GKPGSGKPGSGKPGS

SEQ ID NO: 180 - protein, 20 aa, Artificial Sequence
  Synthetic positively charged scFv linker - (+H)
GKPGSGKPGSGKPGSGKPGS

SEQ ID NO: 181 - protein, 20 aa, Artificial Sequence
  Synthetic positively charged scFv linker - (+I)
GKGKSGKGKSGKGKSGKGKS

SEQ ID NO: 182 - protein, 20 aa, Artificial Sequence
  Synthetic negatively charged scFv linker - Gly-Ser 15
GGGGSGGGGSGGGGSGGGGS

SEQ ID NO: 183 - protein, 14 aa, Artificial Sequence
  Synthetic negatively charged scFv linker - 3hsc_2 (-A)
STAGDTHLGGEDFD

SEQ ID NO: 184 - protein, 15 aa, Artificial Sequence
  Synthetic negatively charged scFv linker - 3hsc_2 (-B)
GEGGSGEGGSGEGGS

SEQ ID NO: 185 - protein, 15 aa, Artificial Sequence
  Synthetic negatively charged scFv linker - 3hsc_2 (-C)
GGEGSGGEGSGGEGS

SEQ ID NO: 186 - protein, 15 aa, Artificial Sequence
  Synthetic negatively charged scFv linker - 3hsc_2 (-D)
GGGESGGGESGGGES

SEQ ID NO: 187 - protein, 15 aa, Artificial Sequence
  Synthetic negatively charged scFv linker - 3hsc_2 (-E)
GEGESGEGESGEGES

SEQ ID NO: 188 - protein, 15 aa, Artificial Sequence
  Synthetic negatively charged scFv linker - 3hsc_2 (-F)
GGGESGGEGSGEGGS

SEQ ID NO: 189 - protein, 20 aa, Artificial Sequence
  Synthetic negatively charged scFv linker - 3hsc_2 (-G)
GEGESGEGESGEGESGEGES

SEQ ID NO: 190 - protein, 8 aa, Artificial Sequence
  Synthetic scFv linker
GGGSGGGS

SEQ ID NO: 191 - protein, 9 aa, Artificial Sequence
  Synthetic scFv linker
GGGGSGGGS

SEQ ID NO: 192 - protein, 9 aa, Artificial Sequence
  Synthetic scFv linker
GGGSGGGGS

SEQ ID NO: 193 - protein, 10 aa, Artificial Sequence
  Synthetic scFv linker
GGGGSGGGGS

SEQ ID NO: 194 - protein, 15 aa, Artificial Sequence
  Synthetic scFv linker
GGGGSGGGGSGGGGS

SEQ ID NO: 195 - protein, 20 aa, Artificial Sequence
  Synthetic scFv linker
GGGGSGGGGSGGGGSGGGGS

SEQ ID NO: 196 - protein, 18 aa, Artificial Sequence
  Synthetic scFv linker
GSTSGSGKPGSGEGSTKG

SEQ ID NO: 197 - protein, 18 aa, Artificial Sequence
  Synthetic scFv linker
GSTSGSGKPGSGEGSTKG

SEQ ID NO: 198 - protein, 18 aa, Artificial Sequence
  Synthetic scFv linker
GSTSGSGKPGSGEGSTKG

SEQ ID NO: 199 - protein, 19 aa, Artificial Sequence
  Synthetic scFv linker
PRGASKSGSASQTGSAPGS

SEQ ID NO: 200 - protein, 19 aa, Artificial Sequence
  Synthetic scFv linker
GTAAAGAGAAGGAAAGAAG

SEQ ID NO: 201 - protein, 19 aa, Artificial Sequence
  Synthetic scFv linker
GTSGSSGSGSGGSGSGGGG

SEQ ID NO: 202 - protein, 20 aa, Artificial Sequence
  Synthetic scFv linker
GKPGSGKPGSGKPGSGKPGS

SEQ ID NO: 203 - protein, 109 aa, Artificial Sequence
  Synthetic construct - exemplary inactive anti-CD3 variable light domain (alpha-CD3 scFv domain)
QTVVTQEPSLTVSPGGTVTLTCGSSTGAVTSGHYPNWVQQKPGQAPRGLIGGTSNKHSWTPARFSGSLLGGKAALTLSGVQPEDEAEYYCVLWGSRRWVFGGGTKLTVL

SEQ ID NO: 204 - protein, 14 aa, Artificial Sequence
  Synthetic construct - exemplary aVLiGLCDR1 of inactive anti-CD3 variable light domain (alpha-CD3 scFv domain)
GSSTGAVTSGHYPN

SEQ ID NO: 205 - protein, 7 aa, Artificial Sequence
  Synthetic construct - exemplary aVLiGLCDR2 of inactive anti-CD3 variable light domain (alpha-CD3 scFv domain)
GTSNKHS

SEQ ID NO: 206 - protein, 9 aa, Artificial Sequence
  Synthetic construct - exemplary aVLiGLCDR3 of inactive anti-CD3 variable light domain (alpha-CD3 scFv domain)
VLWGSRRWV

SEQ ID NO: 207 - protein, 125 aa, Artificial Sequence
  Synthetic construct - exemplary inactive anti-CD3 variable heavy domain (alpha-CD3 scFv domain)
EVQLVESGGGLVQPGGSLKLSCAASGFTFSGYAMNWVRQAPGKGLEWVARIRSKANSYATEYAASVKDRFTISRDDSKNTAYLQMNNLKTEDTAVYYCVRHGNAGNSAISYWAYWGQGTLVTVSS

SEQ ID NO: 208 - protein, 10 aa, Artificial Sequence
  Synthetic construct - exemplary aVHiGL4CDR1 of inactive anti-CD3 variable heavy domain (alpha-CD3 scFv domain)
GFTFSGYAMN

SEQ ID NO: 209 - protein, 19 aa, Artificial Sequence
  Synthetic construct - exemplary aVHiGL4CDR2 of inactive anti-CD3 variable heavy domain (alpha-CD3 scFv domain)
RIRSKANSYATEYAASVKD

SEQ ID NO: 210 - protein, 14 aa, Artificial Sequence
  Synthetic construct - exemplary aVHiGL4CDR3 of inactive anti-CD3 variable heavy domain (alpha-CD3 scFv domain)
HGNAGNSAISYWAY

(19) United States
(12) Patent Application Publication
(10) Pub. No.: US 2021/0309756 A1
(43) Pub. Date: Oct. 7, 2021

(54) COEXPRESSION AND PURIFICATION METHOD OF CONDITIONALLY ACTIVATED BINDING PROTEINS

(71) Applicant: MAVERICK THERAPEUTICS, INC., Brisbane, CA (US)

(72) Inventors: Robert B. DuBridge, Belmont, CA (US); Maia VINOGRADOVA, Moraga, CA (US); Ying ZHU, Brisbane, CA (US)

(21) Appl. No.: 17/266,065

(22) PCT Filed: Aug. 9, 2019
(86) PCT No.: PCT/US2019/045918

§ 371 Date: Feb. 4, 2021

Related U.S. Application Data
(60) Provisional application No. 62/716,755, filed on Aug. 9, 2018.

(51) Int. Cl. C07K 16/30; C07K 16/28

(52) U.S. Cl. CPC C07K 16/30; C07K 16/2827; C07K 16/2863; C07K 2319/50; C07K 2317/569; C07K 2317/94; C07K 2317/62; C07K 2317/31

(57) ABSTRACT

Provided herein are methods for co-expressing and purifying conditionally activated binding proteins such as hemi-COBRAs.

### CROSS REFERENCE TO RELATED APPLICATIONS

This application claims priority to U.S. Provisional Patent Application No. 62/716,755 filed Aug. 9, 2018, all of which is expressly incorporated herein by reference in its entirety.

### REFERENCE TO A “SEQUENCE LISTING,” A TABLE, OR A COMPUTER PROGRAM LISTING APPENDIX SUBMITTED ON A COMPACT DISK

The sequence listing contained in the file named “118459-5006_ST25.txt” and having a size of 183 kilobytes, has been submitted electronically herewith via EFS-Web, and the contents of the txt file are hereby incorporated by reference in their entirety.

### BACKGROUND OF THE INVENTION

The selective destruction of an individual cell or a specific cell type is often desirable in a variety of clinical settings. For example, it is a primary goal of cancer therapy to specifically destroy tumor cells, while leaving healthy cells and tissues as intact and undamaged as possible. One such method is by inducing an immune response against the tumor, to make immune effector cells such as natural killer (NK) cells or cytotoxic T lymphocytes (CTLs) attack and destroy tumor cells.

The use of intact monoclonal antibodies (MAb), which provide superior binding specificity and affinity for a tumor-associated antigen, have been successfully applied in the area of cancer treatment and diagnosis. However, the large size of intact MAbs, their poor bio-distribution and long persistence in the blood pool have limited their clinical applications. For example, intact antibodies can exhibit specific accumulation within the tumor area. In biodistribution studies, an inhomogeneous antibody distribution with primary accumulation in the peripheral regions is noted when precisely investigating the tumor. Due to tumor necrosis, inhomogeneous antigen distribution and increased interstitial tissue pressure, it is not possible to reach central portions of the tumor with intact antibody constructs. In contrast, smaller antibody fragments show rapid tumor localization, penetrate deeper into the tumor, and also, are removed relatively rapidly from the bloodstream.

Single chain fragments (scFv) derived from the small binding domain of the parent MAb offer better biodistribution than intact MAbs for clinical application, and can target tumor cells more efficiently. Single chain fragments can be efficiently engineered from bacteria, however, most engineered scFv have a monovalent structure and show decreased tumor accumulation e.g., a short residence time on a tumor cell, and specificity as compared to their parent MAb ((C(c),D). due to the lack of avidity that bivalent compounds experience.

Despite the favorable properties of scFv, certain features hamper their full clinical deployment in cancer chemotherapy. Of particular note is their cross-reactivity between diseased and healthy tissue due to the targeting of these agents to cell surface receptors common to both diseased and healthy tissue. ScFvs with an improved therapeutic index would offer a significant advance in the clinical utility of these agents. The present invention provides such improved scFvs and methods of manufacturing and using the same. The improved scFvs of the invention have the unexpected benefit of overcoming the lack of avidity demonstrated by a single unit by forming a dimeric compound.

### SUMMARY OF THE INVENTION

In one aspect, provided herein is an isolated cell comprising:

(a) a first polynucleotide encoding a first polypeptide comprising, from N- to C-terminal: (i) a first single domain antibody (sdAb) that binds to a human tumor target antigen (TTA); (ii) a first domain linker; (iii) a variable heavy chain comprising vhCDR1, vhCDR2, and vhCDR3; (iv) a first cleavable linker comprising a first protease cleavage site; and (v) a pseudo variable light chain; and

(b) a second polynucleotide encoding a second polypeptide comprising, from N- to C-terminal: (i) a second sdAb that binds to a human tumor target antigen (TTA); (ii) a second domain linker; (iii) a variable light chain comprising vlCDR1, vlCDR2, and vlCDR3; (iv) a second cleavable linker comprising a second protease cleavage site; and (v) a pseudo variable heavy chain; wherein the variable heavy chain of the first polypeptide and the variable light chain of said second polypeptide bind human CD3 when associated to form a Fv.

In some embodiments, the first sdAb and the second sdAb bind to the same human TTA. In some instances, the first sdAb and the second sdAb comprise the same amino acid sequence. In other instances, said first sdAb and said second sdAb comprise different amino acid sequences. In certain embodiments, the first sdAb and the second sdAb bind to different human TTAs.

In some embodiments, the first sdAb and/or the second sdAb bind a human TTA selected from the group consisting of human EGFR, human B7H3, human EpCAM, and human FOLR1.

In some embodiments of any of the inventions described herein, the first and second sdAbs bind to human EGFR. In certain embodiments, the first and second sdAbs bind to human B7H3. In particular embodiments, the first and second sdAbs bind to human EpCAM. In other embodiments, the first and second sdAbs bind to and human FOLR1. In some embodiments, the first sdAb binds to human EGFR and the second sdAb binds to human B7H3.

In some embodiments of any of the inventions described herein, the first sdAb binds to human EGFR and the second sdAb binds to human EpCAM. In some embodiments, the first sdAb binds to human EGFR and the second sdAb binds to human FOLR1. In some embodiments, the first sdAb binds to human B7H3 and the second sdAb binds to human EGFR. In some embodiments, the first sdAb binds to human EpCAM and the second sdAb binds to human EGFR. In some embodiments, the first sdAb binds to human FOLR1 and the second sdAb binds to human EGFR. In some embodiments, the first sdAb binds to human B7H3 and the second sdAb binds to human EpCAM. In some embodiments, the first sdAb binds to human FOLR1 and the second sdAb binds to human EpCAM. In some embodiments, the first sdAb binds to human B7H3 and the second sdAb binds to human FOLR1. In some embodiments, the first sdAb binds to human EpCAM and the second sdAb binds to human B7H3. In some embodiments, the first sdAb binds to human EpCAM and the second sdAb binds to human FOLR1. In some embodiments, the first sdAb binds to human FOLR1 and the second sdAb binds to human B7H3. In some embodiments, the first sdAb binds to human FOLR1 and the second sdAb binds to human EpCAM.

In some embodiments, the first and second protease cleavage sites are recognized by the same protease. In other embodiments, the first and second protease cleavage sites are recognized by different proteases.

In some embodiments, the first polypeptide further comprises a half life extension domain at the C-terminal end and/or the second polypeptide further comprises a half life extension domain at the C-terminal end. In another way, the first polypeptide further comprises a half life extension domain at the C-terminal end and the second polypeptide further comprises a half life extension domain at the C-terminal end. In some cases, the first polypeptide further comprises a half life extension domain at the C-terminal end. In some instances, the second polypeptide further comprises a half life extension domain at the C-terminal end.

In some embodiments, the variable heavy chain of the first polypeptide comprises the vhCDR1, vhCDR2, and vhCDR3 sequence of SEQ ID NO:102 of FIG. 39. In some embodiments, the pseudo variable heavy chain of the first polypeptide comprises the pseudo variable heavy chain sequence of any one selected from the group consisting of SEQ ID NO:106, SEQ ID NO:110, and SEQ ID NO:207 of FIG. 39. In some embodiments, the pseudo variable heavy chain comprises the ivhCDR1, ivhCDR2, and ivhCDR3 sequences of SEQ ID NO:107, SEQ ID NO:108, SEQ ID NO:109 of FIG. 39, respectively. In other embodiments, the pseudo variable heavy chain comprises the ivhCDR1, ivhCDR2, and ivhCDR3 sequences of SEQ ID NO:111, SEQ ID NO:112, SEQ ID NO:103 of FIG. 39, respectively. In some embodiments, the pseudo variable heavy chain comprises the ivhCDR1, ivhCDR2, and ivhCDR3 sequences of SEQ ID NO:208, SEQ ID NO:209, SEQ ID NO:210 of FIG. 39, respectively. In some embodiments, the variable light chain of the second polypeptide comprises the vlCDR1, vlCDR2, and vlCDR3 sequence of SEQ ID NO:90 of FIG. 38. In some embodiments, the pseudo variable light chain of the second polypeptide comprises the pseudo variable light chain sequence of any one selected from the group consisting of SEQ ID NO:94, SEQ ID NO:98, and SEQ ID NO:203 of FIG. 38. In some embodiments, the pseudo variable light chain comprises the ivlCDR1, ivlCDR2, and ivlCDR3 sequences of SEQ ID NO:95, SEQ ID NO:96, SEQ ID NO:97 of FIG. 38, respectively. In other embodiments, the pseudo variable light chain comprises the ivlCDR1, ivlCDR2, and ivlCDR3 sequences of SEQ ID NO:99, SEQ ID NO:100, SEQ ID NO:101 of FIG. 38, respectively. In some embodiments, the pseudo variable light chain comprises the ivlCDR1, ivlCDR2, and ivlCDR3 sequences of SEQ ID NO:204, SEQ ID NO:205, SEQ ID NO:206 of FIG. 38, respectively.

In some embodiments, the first polypeptide is selected from the group consisting of Pro16 (SEQ ID NO:5), Pro39 (SEQ ID NO:9), Pro41 (SEQ ID NO:13), Pro43 (SEQ ID NO:17), Pro45 (SEQ ID NO:21), and Pro349 (SEQ ID NO:25). In some embodiments, the second polypeptide is selected from the group consisting of Pro19 (SEQ ID NO:7), Pro40 (SEQ ID NO:11), Pro42 (SEQ ID NO:15), Pro44 (SEQ ID NO:19), Pro46 (SEQ ID NO:23), and Pro353 (SEQ ID NO:27). In some embodiments, the said first polypeptide and said second polypeptide are selected from the group consisting of Pro16+Pro19 (SEQ ID NO:5 and SEQ ID NO:7), Pro39+Pro40 (SEQ ID NO:9 and SEQ ID NO:11), Pro41+Pro42 (SEQ ID NO:13 and SEQ ID NO:15), Pro43+Pro44 (SEQ ID NO:17 and SEQ ID NO:19), Pro45+Pro46 (SEQ ID NO:21 and SEQ ID NO:23), and Pro349+Pro353 (SEQ ID NO:25 and SEQ ID NO:27).

In some embodiments, the first polynucleotide and the second polynucleotide are introduced into said cell in different expression vectors. In some embodiments, the first polynucleotide and the second polynucleotide are introduced into the cell described herein at a polynucleotide ratio to produce substantially equivalent amounts of the first polypeptide and the second polypeptide. In some instances, the ratio (the polynucleotide ratio) of the first polynucleotide to the second polynucleotide is 1:1. In some instances, the ratio of the first polynucleotide to the second polynucleotide is greater than 1:1. In certain instances, the ratio of the first polynucleotide to the second polynucleotide is less than 1:1.

Also, provided in the invention is an expression vector comprising any one of the first polynucleotides outlined herein. Similarly, provided is an expression vector comprising any one of the second polynucleotides outlined herein.

In another aspect, the invention discloses a composition comprising the first expression vector and the second expression vector outlined herein, wherein the first expression vector and the second expression vector are introduced into a host cell at a polynucleotide ratio to produce substantially equivalent amounts of said first polypeptide and said second polypeptide. In some embodiments, the ratio of the first expression vector to the second expression vector is 1:1. In other embodiments, the ratio of the first expression vector to the second expression vector is greater than 1:1. In other embodiments, the ratio of the first expression vector to the second expression vector is less than 1:1.

In an aspect, the invention provides a method of isolating a prodrug composition comprising a first polypeptide and a second polypeptide. The method comprises: (1) culturing a host cell under suitable culture conditions to produce and secrete a first polypeptide and a second polypeptide into culture media; and (2) purifying the first polypeptide and the second polypeptide from the culture media using Protein A chromatography, thereby isolating a prodrug composition comprising a first polypeptide and a second polypeptide; wherein the host cell comprises:

(a) a first polynucleotide encoding the first polypeptide comprising, from N- to C-terminal: (i) a first sdAb that binds to a human tumor target antigen (TTA); (ii) a first domain linker; (iii) a variable heavy chain comprising vhCDR1, vhCDR2, and vhCDR3; (iv) a first cleavable linker comprising a first protease cleavage site; and

(b) a second polynucleotide sequence encoding a second polypeptide comprising, from N- to C-terminal: (i) a second sdAb that binds to a human tumor target antigen (TTA); (ii) a second domain linker; (iii) a variable light chain comprising vlCDR1, vlCDR2, and vlCDR3; (iv) a second cleavable linker comprising a second protease cleavage site; and (v) a pseudo variable heavy chain; wherein the variable heavy chain of the first polypeptide and the variable light chain of the second polypeptide bind human CD3 when associated to form an Fv.

In some embodiments, the first polypeptide and the second polypeptide are purified separately. In other embodiments, the first polypeptide and the second polypeptide are purified simultaneously.

In some embodiments, purifying further comprises performing affinity chromatography after the Protein A chromatography.

In some embodiments, the prodrug composition comprises a substantially equivalent amount of the first polypeptide and the second polypeptide.

In some embodiments, the first sdAb and the second sdAb bind to the same human TTA. In some instances, the first sdAb and the second sdAb comprise the same amino acid sequence. In other instances, said first sdAb and said second sdAb comprise different amino acid sequences. In certain embodiments, the first sdAb and the second sdAb bind to different human TTAs.

In some embodiments, the first sdAb and/or the second sdAb bind a human TTA selected from the group consisting of human EGFR, human B7H3, human EpCAM, and human FOLR1.

In some embodiments, the first and second protease cleavage sites are recognized by the same protease. In certain embodiments, the first and second protease cleavage sites are recognized by different proteases.

In some embodiments, the first polypeptide comprises a half life extension domain at the C-terminal end and/or the second polypeptide comprises a half life extension domain at the C-terminal end. In some instances, the first polypeptide further comprises a half life extension domain at the C-terminal end and the second polypeptide further comprises a half life extension domain at the C-terminal end. In some cases, the first polypeptide further comprises a half life extension domain at the C-terminal end. In other cases, the second polypeptide further comprises a half life extension domain at the C-terminal end.

In some embodiments, the variable heavy chain of the first polypeptide comprises the vhCDR1, vhCDR2, and vhCDR3 sequence of SEQ ID NO:102 of FIG. 39. In some embodiments, the pseudo variable heavy chain comprises the pseudo variable heavy chain sequence of any one selected from the group consisting of SEQ ID NO:106, SEQ ID NO:110, and SEQ ID NO:207 of FIG. 39. In some embodiments, the pseudo variable heavy chain comprises the ivhCDR1, ivhCDR2, and ivhCDR3 sequences of SEQ ID NO:107, SEQ ID NO:108, SEQ ID NO:109 of FIG. 39, respectively. In other embodiments, the pseudo variable heavy chain comprises the ivhCDR1, ivhCDR2, and ivhCDR3 sequences of SEQ ID NO:111, SEQ ID NO:112, SEQ ID NO:103 of FIG. 39, respectively. In some embodiments, the pseudo variable heavy chain comprises the ivhCDR1, ivhCDR2, and ivhCDR3 sequences of SEQ ID NO:208, SEQ ID NO:209, SEQ ID NO:210 of FIG. 39, respectively. In some embodiments, the variable light chain of the second polypeptide comprises the vlCDR1, vlCDR2, and vlCDR3 sequence of SEQ ID NO:90 of FIG. 38. In some embodiments, the pseudo variable light chain comprises the pseudo variable light chain sequence of any one selected from the group consisting of SEQ ID NO:94, SEQ ID NO:98, and SEQ ID NO:203 of FIG. 38. In some embodiments, the pseudo variable light chain comprises the ivlCDR1, ivlCDR2, and ivlCDR3 sequences of SEQ ID NO:95, SEQ ID NO:96, SEQ ID NO:97 of FIG. 38, respectively. In other embodiments, the pseudo variable light chain comprises the ivlCDR1, ivlCDR2, and ivlCDR3 sequences of SEQ ID NO:99, SEQ ID NO:100, SEQ ID NO:101 of FIG. 38, respectively. In some embodiments, the pseudo variable light chain comprises the ivlCDR1, ivlCDR2, and ivlCDR3 sequences of SEQ ID NO:204, SEQ ID NO:205, SEQ ID NO:206 of FIG. 38, respectively.

In some embodiments, the first polypeptide is selected from the group consisting of Pro16 (SEQ ID NO:5), Pro39 (SEQ ID NO:9), Pro41 (SEQ ID NO:13), Pro43 (SEQ ID NO:17), Pro45 (SEQ ID NO:21), and Pro349 (SEQ ID NO:25). In some embodiments, the second polypeptide is selected from the group consisting of Pro19 (SEQ ID NO:7), Pro40 (SEQ ID NO:11), Pro42 (SEQ ID NO:15), Pro44 (SEQ ID NO:19), Pro46 (SEQ ID NO:23), and Pro353 (SEQ ID NO:27). In some embodiments, the said first polypeptide and said second polypeptide are selected from the group consisting of Pro16+Pro19 (SEQ ID NO:5 and SEQ ID NO:7), Pro39+Pro40 (SEQ ID NO:9 and SEQ ID NO:11), Pro41+Pro42 (SEQ ID NO:13 and SEQ ID NO:15), Pro43+Pro44 (SEQ ID NO:17 and SEQ ID NO:19), Pro45+Pro46 (SEQ ID NO:21 and SEQ ID NO:23), and Pro349+Pro353 (SEQ ID NO:25 and SEQ ID NO:27).

In some embodiments, the first polynucleotide and said second polynucleotide are introduced into the host cell in different expression vectors. In some embodiments, the first polynucleotide and the second polynucleotide have been introduced into the host cell in a single expression vector.

The method of isolating any one of the prodrug compositions described can further comprise introducing the first polynucleotide and the second polynucleotide into a host cell at at a polynucleotide ratio to produce substantially equivalent amounts the first polypeptide and the second polypeptide. In some embodiments, the first polynucleotide and said second polynucleotide are introduced into said host cell in different expression vectors or in a single expression vector.

In some embodiments, the first polynucleotide and the second polynucleotide are introduced into the host cell at a polynucleotide ratio to produce substantially equivalent amounts the first polypeptide and the second polypeptide. In some embodiments, the polynucleotide ratio of the first polynucleotide to the second polynucleotide is 1:1. In certain embodiments, the polynucleotide ratio of the first polynucleotide to the second polynucleotide is greater than 1:1. In other embodiments, the polynucleotide ratio of the first polynucleotide to the second polynucleotide is less than 1:1.

Provided herein is a method of treating cancer in a human subject in need thereof comprising administering the prodrug composition produced according to the any one of the methods outlined herein.

Reference is made to WO 2019/051122, filed on Sep. 6, 2018, U.S. Provisional Application No. 62/555,999, filed on Sep. 8, 2017, WO 2019/051102, filed on Sep. 6, 2018, U.S. Provisional Application No. 62/555,943, filed on Sep. 8, 2017, U.S. Provisional Application No. 62/586,627, filed on Nov. 15, 2017, U.S. Provisional Application No. 62/587,318, filed on Nov. 16, 2017, WO2017/156178, filed on Mar. 8, 2017, U.S. Provisional Application No. 62/555,999, filed on Sep. 8, 2017, which are expressly incorporated by reference in their entirety, including the Figures, Legends, and definitions, as well as all recited embodiments.

### BRIEF DESCRIPTION OF THE DRAWINGS

FIG. 1A and FIG. 1B depict a schematic diagram of exemplary hemi-COBRA constructs. The structure of the Pro16 construct includes (from the N-terminal end to C-terminal end) an anti-EGFR1 sdAb-a domain linker-an active variable heavy domain of an anti-CD3 scFv-a cleavable linker-a pseudo (inactive) variable light chain of an anti-CD3 scFv-a half life extension domain (e.g., anti-HSA). The structure of the Pro19 construct includes (from the N-terminal end to C-terminal end) an anti-EGFR2 sdAb-a domain linker—an active variable light chain of an anti-CD3 scFv-a cleavable linker-a pseudo variable heavy chain of an anti-CD3 scFv-a half life extension domain (e.g., anti-HSA). Uncleaved molecules bind to EGFR, do not bind to CD3, and are not active in T cell cytotoxicity assays. A protease (e.g., enterokinase) that recognizes the protease cleavage site of the cleavable linker can cleave the Pro16 and Pro19 hemi-COBRAs to produce an active anti-CD3 Fv molecule as the intact VH domain and the intact VL domain are both tethered to a cancer cell through the anti-EGFR1/2 sdAbs of the active construct. FIG. 1B depict generic hemi-COBRA constructs. The structure of the Pro1 construct includes (from the N-terminal end to C-terminal end) an anti-TTA1 sdAb-a domain linker-an active variable heavy domain of an anti-CD3 scFv-a cleavable linker-a pseudo (inactive) variable light chain of an anti-CD3 scFv-a half life extension domain (e.g., anti-HSA). The structure of the Pro2 construct includes (from the N-terminal end to C-terminal end) an anti-TTA2 sdAb-a domain linker-an active variable light chain of an anti-CD3 scFv-a cleavable linker-a pseudo variable heavy chain of an anti-CD3 scFv-a half life extension domain (e.g., anti-HSA). In some cases, the anti-TTA1 and anti-TTA2 bind the same tumor antigen. In other cases, the anti-TTA1 and anti-TTA2 bind different tumor antigens.

FIG. 2 shows that EK cleavage co-operatively activates T cell killing of EGFR+ target cells with complementary hemi-COBRAs Pro16 and Pro19. Pro51 represents a positive control for T cell dependent cytotoxicity.

FIG. 3 shows SDS-PAGE analysis of co-expressed hemi-COBRAs before and after proteolytic cleavage. The hemi-COBRAs were co-expressed from transiently co-transfected population of expi293 cells, and then co-purified from the resulting conditioned media using a Protein A purification column. The data shows that some hemi-COBRA pairs co-express equivalently (such as Pro45/Pro46) and others hemi-COBRA pairs do not (such as Pro43/Pro44). Additional experiments showed that adjusting the expression vector ratios of each hemi-COBRA in the co-transfection resulted in equivalent expression of the hemi-COBRA pair.

FIG. 4A and FIG. 4B show the main products of co-expression and co-purification of the Pro16 and Pro19 constructs are equivalent to a monomeric hemi-COBRA (Pro19 monomer), as measured by analytical size exclusion chromatography (SEC). The data shows that co-expression and co-purification of the Pro16 and Pro19 constructs does not result in protein aggregation.

FIG. 5 shows SEC analysis of co-expressed and co-purified of Pro16 and Pro19 compared to an equimolar mixture of each monomer.

FIG. 6A and FIG. 6B show that the Pro16 construct results in a monomer peak on analytical SEC. FIG. 6B shows a comparison of the Pro16 construct relative to the Pro19 monomer.

FIG. 7A and FIG. 7B show the main products of co-expression and co-purification of the Pro39 and Pro40 constructs are equivalent to a monomeric hemi-COBRA (Pro19 monomer), as measured by analytical SEC.

FIG. 8 shows SEC analysis of co-expressed and co-purified Pro39 and Pro40 constructs compared to an equimolar mixture of each monomer.

FIG. 9A and FIG. 9B show the main products of co-expression and co-purification of the Pro41 and Pro42 constructs are equivalent to a monomeric hemi-COBRA (Pro19 monomer), as measured by analytical SEC.

FIG. 10 shows SEC analysis of co-expressed and co-purified Pro41 and Pro42 constructs compared to an equimolar mixture of each monomer.

FIG. 11A and FIG. 11B show the main products of co-expression and co-purification of the Pro43 and Pro44 constructs are equivalent to a monomeric hemi-COBRA (Pro19 monomer), as measured by analytical SEC.

FIG. 12A and FIG. 12B show the main products of co-expression and co-purification of the Pro45 and Pro46 constructs are equivalent to a monomeric hemi-COBRA (Pro19 monomer), as measured by analytical SEC.

FIG. 13A, FIG. 13B, FIG. 13C, FIG. 13D, FIG. 13E, and FIG. 13F show TDCC assays with co-expressed hemi-COBRAs. The T cell killing assays demonstrate that the co-expressed hemi-COBRA pairs exhibit expected levels of potency consistent with the lower expressed hemi-COBRA for each pair. FIG. 13A depicts results of the cleaved Pro16 and Pro19 pair. FIG. 13B depicts results of the cleaved Pro39 and Pro40 pair. FIG. 13C depicts results of the cleaved Pro41 and Pro42 pair. FIG. 13D depicts results of the cleaved Pro43 and Pro44 pair. FIG. 13E depicts results of the cleaved Pro45 and Pro46 pair. FIG. 13F depicts results of the cleaved Pro59 and Pro60 pair. Pro51 represents a positive control for T cell dependent cytotoxicity.

FIG. 14A, FIG. 14B, and FIG. 14C provide a number of suitable protease cleavage sites. As will be appreciated by those in the art, these cleavable sites can be used as cleavable linkers. In some embodiments, for example, when more flexible cleavable linkers are required, there can be additional amino acids (generally glycines and serines) that are either or both N-terminal and C-terminal to these cleavage sites.

FIG. 15 depicts exemplary linker sequences such as domain linker sequences for use in embodiments described herein.

FIG. 16A and FIG. 16B depict an exemplary anti-EGFR hemi-COBRA. FIG. 16A-FIG. 16B show the amino acid sequence (SEQ ID NO:1) and the nucleic acid sequence (SEQ ID NO:2) of Pro6, respectively. Pro6 and Pro7 can form a hemi-COBRA pair.

FIG. 17A and FIG. 17B depict an exemplary anti-EGFR hemi-COBRA. FIG. 17A-FIG. 17B show the amino acid sequence (SEQ ID NO:3) and the nucleic acid sequence (SEQ ID NO:4) of Pro7, respectively.

FIG. 18A and FIG. 18B depict an exemplary anti-EGFR hemi-COBRA. FIG. 18A-FIG. 18B show the amino acid sequence (SEQ ID NO:5) and the nucleic acid sequence (SEQ ID NO:6) of Pro16, respectively. Pro16 and Pro19 can form a hemi-COBRA pair.

FIG. 19A and FIG. 19B depict an exemplary anti-EGFR hemi-COBRA. FIG. 19A-FIG. 19B show the amino acid sequence (SEQ ID NO:7) and the nucleic acid sequence (SEQ ID NO:8) of Pro19, respectively.

FIG. 20A and FIG. 20B depict an exemplary anti-EGFR hemi-COBRA. FIG. 20A-FIG. 20B show the amino acid sequence (SEQ ID NO:9) and the nucleic acid sequence (SEQ ID NO:10) of Pro39, respectively. Pro39 and Pro40 can form a hemi-COBRA pair.

FIG. 21A and FIG. 21B depict an exemplary anti-EGFR hemi-COBRA. FIG. 21A-FIG. 21B show the amino acid sequence (SEQ ID NO:11) and the nucleic acid sequence (SEQ ID NO:12) of Pro40, respectively.

FIG. 22A and FIG. 22B depict an exemplary anti-EGFR hemi-COBRA. FIG. 22A-FIG. 22B show the amino acid sequence (SEQ ID NO:13) and the nucleic acid sequence (SEQ ID NO:14) of Pro41, respectively. Pro41 and Pro42 can form a hemi-COBRA pair.

FIG. 23A and FIG. 23B depict an exemplary anti-EGFR hemi-COBRA. FIG. 23A-FIG. 23B show the amino acid sequence (SEQ ID NO:15) and the nucleic acid sequence (SEQ ID NO:16) of Pro42, respectively.

FIG. 24A and FIG. 24B depict an exemplary anti-EGFR hemi-COBRA. FIG. 24A-FIG. 24B show the amino acid sequence (SEQ ID NO:17) and the nucleic acid sequence (SEQ ID NO:18) of Pro43, respectively. Pro43 and Pro44 can form a hemi-COBRA pair.

FIG. 25A and FIG. 25B depict an exemplary anti-EGFR hemi-COBRA. FIG. 25A-FIG. 25B show the amino acid sequence (SEQ ID NO:19) and the nucleic acid sequence (SEQ ID NO:20) of Pro44, respectively.

FIG. 26A and FIG. 26B depict an exemplary anti-EGFR hemi-COBRA. FIG. 26A-FIG. 26B show the amino acid sequence (SEQ ID NO:21) and the nucleic acid sequence (SEQ ID NO:22) of Pro45, respectively. Pro45 and Pro46 can form a hemi-COBRA pair.

FIG. 27A and FIG. 27B depict an exemplary anti-EGFR hemi-COBRA. FIG. 27A-FIG. 27B show the amino acid sequence (SEQ ID NO:23) and the nucleic acid sequence (SEQ ID NO:24) of Pro46, respectively.

FIG. 28A and FIG. 28B depict an exemplary anti-EGFR hemi-COBRA. FIG. 28A-FIG. 28B show the amino acid sequence (SEQ ID NO:25) and the nucleic acid sequence (SEQ ID NO:26) of Pro349, respectively. Pro349 and Pro353 can form a hemi-COBRA pair.

FIG. 29A and FIG. 29B depict an exemplary anti-EGFR hemi-COBRA. FIG. 29A-FIG. 29B show the amino acid sequence (SEQ ID NO:27) and the nucleic acid sequence (SEQ ID NO:28) of Pro353, respectively.

FIG. 30A and FIG. 30B depict an exemplary anti-B7H3 hemi-COBRA. FIG. 30A-FIG. 30B show the amino acid sequence (SEQ ID NO:29) and the nucleic acid sequence (SEQ ID NO:30) of Pro348, respectively. Pro348 and Pro352 can form a hemi-COBRA pair.

FIG. 31A and FIG. 31B depict an exemplary anti-B7H3 hemi-COBRA. FIG. 31A-FIG. 31B show the amino acid sequence (SEQ ID NO:31) and the nucleic acid sequence (SEQ ID NO:32) of Pro352, respectively.

FIG. 32A and FIG. 32B depict an exemplary anti-EGFR hemi-COBRA. FIG. 32A-FIG. 32B show the amino acid sequence (SEQ ID NO:23) and the nucleic acid sequence (SEQ ID NO:34) of Pro350, respectively. Pro350 and Pro354 can form a hemi-COBRA pair.

FIG. 33A and FIG. 33B depict an exemplary anti-EGFR hemi-COBRA. FIG. 33A-FIG. 33B show the amino acid sequence (SEQ ID NO:35) and the nucleic acid sequence (SEQ ID NO:36) of Pro354, respectively.

For the antigen binding domains in FIG. 16A-FIG. 16B, FIG. 17A-FIG. 17B, FIG. 18A-FIG. 18B, FIG. 19A-FIG. 19B, FIG. 20A-FIG. 20B, FIG. 21A-FIG. 21B, FIG. 22A-FIG. 22B, FIG. 23A-FIG. 23B, FIG. 24A-FIG. 24B, FIG. 25A-FIG. 25B, FIG. 26A-FIG. 26B, FIG. 27A-FIG. 27B, FIG. 28A-FIG. 28B, FIG. 29A-FIG. 29B, FIG. 30A-FIG. 30B, FIG. 31A-FIG. 31B, FIG. 32A-FIG. 32B, and FIG. 33A-FIG. 33B, the CDRs are bolded and single underlined. Linkers are double underlined. Cleavable linkers are italicized and double underlined. Slashes (“/”) depict domain separators.

FIG. 34 provides amino acid sequences of exemplary TTA ABDs of the invention, such as anti-EGFR sdAbs of SEQ ID NOS:37, 41, 45, and 50. The CDRs are bolded and single underlined and correspond to SEQ ID NO identifiers depicted in the Figure.

FIG. 35 provides amino acid sequences of exemplary TTA ABDs of the invention, such as anti-FOLR1 sdAbs of SEQ ID NOS:54, 58, and 62. The CDRs are bolded and single underlined and correspond to SEQ ID NO identifiers depicted in the Figure.

FIG. 36 provides amino acid sequences of exemplary TTA ABDs of the invention, such as anti-B7H3 sdAbs of SEQ ID NOS:66 and 68, and anti-EpCAM sdAbs of SEQ ID NOS:74 and 78. The CDRs are bolded and single underlined and correspond to SEQ ID NO identifiers depicted in the Figure.

FIG. 37 provides amino acid sequences of exemplary anti-HSA binding domains (half-life extension domains) of the invention such as those of SEQ ID NOS:82 and 86. The CDRs are bolded and single underlined and correspond to SEQ ID NO identifiers depicted in the Figure.

FIG. 38 provides amino acid sequences of exemplary active anti-CD3 variable light domains (e.g., αCD3 VL, SEQ ID NO:90) and exemplary inactive anti-CD3 variable light domains (e.g., αCD3 VLi, SEQ ID NO:94; αCD3 VLi2, SEQ ID NO:98; and αCD3 VLiGL, SEQ ID NO:203) of the invention. The CDRs are bolded and single underlined and correspond to SEQ ID NO identifiers depicted in the Figure.

FIG. 39 provides amino acid sequences of exemplary active anti-CD3 variable heavy domains (e.g., αCD3 VH, SEQ ID NO:102) and exemplary inactive anti-CD3 variable heavy domains (e.g., αCD3 VHi, SEQ ID NO:106; αCD3 VHi2, SEQ ID NO:110; and αCD3 VHiGL4, SEQ ID NO:207) of the invention. The CDRs are bolded and single underlined and correspond to SEQ ID NO identifiers depicted in the Figure.

FIG. 40 provides amino acid sequences of an exemplary anti-CD3 scFv linkers. In one embodiment, the linker is a noncleavable linker having the amino acid sequence of SEQ ID NO:114. In a different embodiment, the linker has the amino acid sequence of SEQ ID NO:115.

FIG. 41 depicts the components for two pairs of hemi-COBRAs (Pro348/Pro352 and Pro350/Pro354). The table also provides the yield of each hemi-COBRA when produced individually by transient transfection of Expi293 cells.

FIG. 42 shows data obtained from plasmid DNA ratio optimization experiments using the hemi-COBRA pairs Pro348/Pro352 and Pro350/Pro354. The solid black line depicts the Pro348/Pro352 as detected via the His6 tag. The solid grey line depicts the Pro348/Pro352 as detected via the Strep2 tag. The hashed black line depicts the Pro350/Pro354 as detected via the Strep2 tag. The hashed grey line depicts the Pro350/Pro354 as detected via the His6 tag.

FIG. 43 shows the optimal plasmid DNA ratio of each member of a hemi-COBRA pair results in a similar productivity of the co-expressed hemi-COBRAs. In some cases, the optimal DNA ratio for the pair Pro348 and Pro352 is 2.5:7.5 (Pro348:Pro352). In some cases, the optimal DNA ratio for the pair Pro350 and Pro354 is 2:8 (Pro350:Pro354).

FIG. 44 shows productivities of stable cell lines expressing a pair of hemi-COBRAs. Four stable clones expressing Pro348/Pro352 (clones 1E9, 1F4, 3B9, and 4B12) were evaluated. Three stable clones expressing Pro350/Pro354 (clones 3C8, 4H3, and 2G2) were evaluated. The amount of each Pro construct produced by clones in different culturing formats (such as 96-well plates, 12-well plates, 24-well plates, and in a suspension culture) was measured by Octet analysis.

FIG. 45A, FIG. 45B, and FIG. 45C show exemplary data from colony screening experiments of stable clones expressing a hemi-COBRA pair such as Pro348/Pro352 or Pro350/Pro354. FIG. 45A represents the clone ID numbers. FIG. 45B shows data using the Octet His1K biosensors to detect His-tagged Pro352 and Pro354. FIG. 45C shows data using the Octet SAX biosensors to detect Strep2-tagged Pro348 and Pro350.

FIG. 46 shows Octet data from 3 top clones from the Pro348/Pro352 (clones 1F4, 3B9, and 4B12) and Pro350/Pro354 (clones 3C8, 4H3, and 2G2) stable cell lines.

### DETAILED DESCRIPTION OF THE INVENTION

### I. Introduction

The present invention is directed to methods of making compositions that are useful in the production of therapeutic moieties. Specifically, the methods of the invention allow the creation of therapeutic proteins that reduce the toxicity and side effects of bispecific antibodies (including antibody-like functional proteins) that bind to important physiological targets such as CD3 and tumor antigens. Many antigen binding proteins, such as antibodies, can have significant off-target side effects, and thus there is a need to only activate the binding capabilities of a therapeutic molecule in the vicinity of the disease tissue, to avoid off-target interactions. Accordingly, the present invention is directed to methods of making multivalent conditionally effective (“MCE”) proteins that have a number of functional protein domains. In general, one of these domains is an antigen binding domain (ABD) that will bind a target tumor antigen (TTA), and another is an ABD that will bind a T-cell antigen such as CD3 under certain conditions. Additionally, the MCE proteins also include one or more protease cleavage sites. That is, the therapeutic molecules are made in a “pro-drug” like format, wherein the CD3 binding domain is inactive until exposed to a tumor environment. The tumor environment has an elevated level of proteolytic activity, such that upon exposure to a protease in the tumor environment, the prodrug is cleaved and becomes active.

This is generally accomplished herein by using proteins that include a “pseudo” variable heavy domain and a “pseudo” variable light domain directed to the T-cell antigen such as CD3, that restrain the CD3 Fvs of the MCE into an inactive format as is discussed herein. As the TTA targets the MCE into the proximity of the tumor, the MCE is thus exposed to the protease. Upon cleavage, the active variable heavy domain and active light domain are now able to pair to form one or more active ABDs to CD3 and thus recruit T cells to the tumor, resulting in treatment.

However, the use of two proteins for use in a single mechanism creates a problem for producing these molecules as a drug. One solution is to make two expression cell lines, each expressing a different hemi-COBRA, then the two hemi-COBRAs can be expressed and purified separately and mixed to produce a pro-drug cocktail. Unfortunately, this is a time-consuming and expensive process to generate large doses of the drug cocktail. Another solution is to co-express the two hemi-COBRAs in the same cell and then purify them either separately or together from the same conditioned media.

Accordingly, the present invention is directed to methods of co-expressing and co-purifying a complementary pair of therapeutic molecules that can bind CD3 and tumor antigens under certain conditions. The method can be used to produce each of the proteins of the complementary pair in an about equimolar ratio. Also provided are stable cell lines expressing the complementary pair of therapeutic molecules.

The present invention is also directed to methods of producing therapeutic molecules that can reduce the toxicity and side effects of bispecific antibodies (including antibody-like functional proteins) that bind to important physiological targets such as CD3 and tumor antigens. Many antigen binding proteins, such as antibodies, can have significant off-target side effects, and thus there is a need to only activate the binding capabilities of a therapeutic molecule in the vicinity of the disease tissue, to avoid off-tumor interactions. Accordingly, the present invention is directed to conditionally effective proteins that have a number of functional protein domains. In general, one of these domains is an antigen binding domain (ABD) that will bind a target tumor antigen (TTA), and another is a portion of an ABD that will bind a T-cell antigen such as CD3 under certain conditions such as when the portion of an ABD is in close proximity to a complementary portion of the ABD to form an anti-CD3 Fv binding domain. That is, the therapeutic molecules are made in a “pro-drug” like format, wherein the CD3 binding domain is inactive until exposed to a tumor environment. As tumor environments overexpress proteases, the pro-drug of the therapeutic molecule undergoes cleavage to produce an active therapeutic molecule.

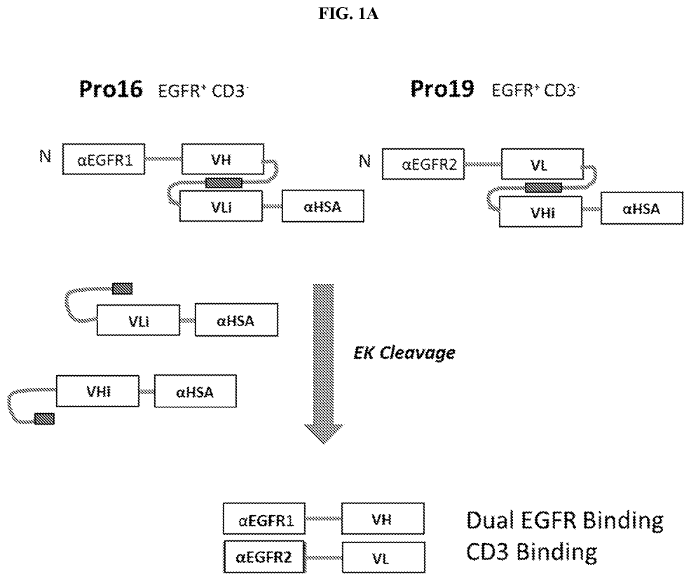

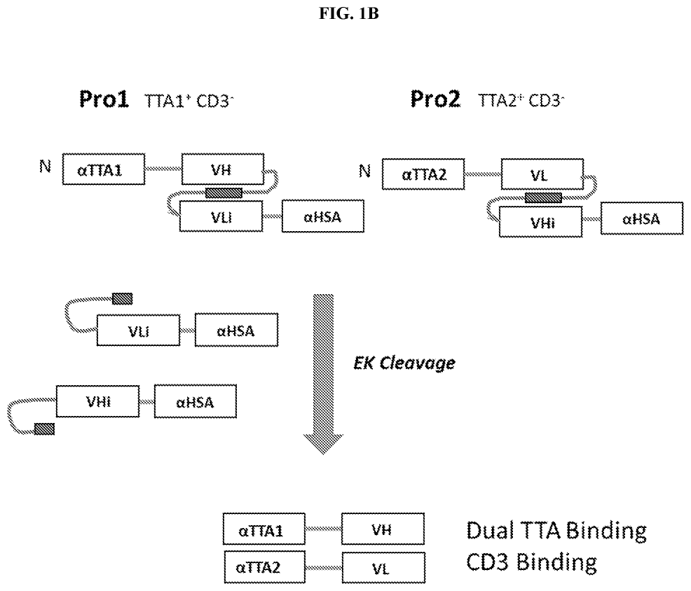

In the present invention, the prodrug (e.g. uncleaved) format, the prodrug polypeptide comprises a “pseudo VL domain” or “pseudo VH domain”. Schematic diagrams of complementary prodrug constructs are shown in FIG. 1. The pseudo variable heavy domain and pseudo variable light domain contain standard framework regions, but “inactive” (“inert” or “dummy”) CDRs. However, due to the “inert” CDRs of the pseudo domain, the resulting ABDs will not bind CD3, thus preventing off tumor toxicities in proteolytically inactive tissues. However, in the presence of proteases that are in or near the tumor, the prodrug constructs are cleaved in such a way as to allow the “real” variable heavy and variable light domains to associate, thus triggering active CD3 binding and the resulting in anti-tumor efficacy.

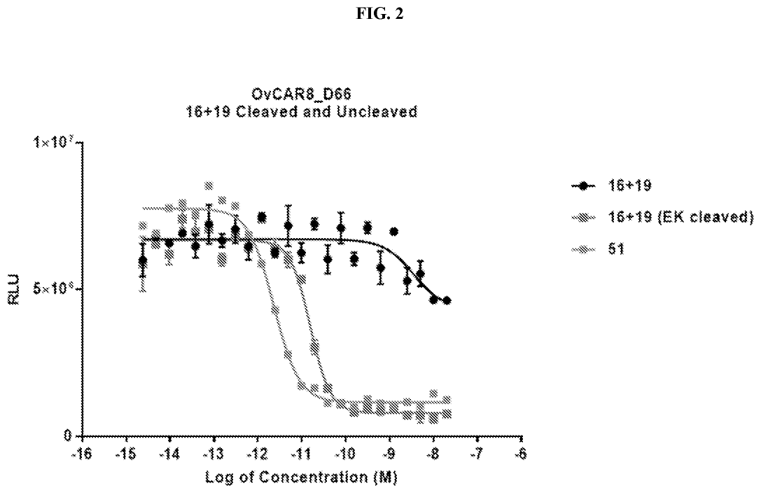

The prodrug activation can happen as is generally shown in FIG. 1 and FIG. 2. In FIG. 1, the prodrug construct Pro16 has one cleavage site (e.g., a FLAG site) between the active VH domain and the pseudo VL domain (also referred to as the VLi domain) and the prodrug construct Pro19 has one cleavage site (e.g., a FLAG site) between the pseudo VH domain (also referred to as the VHi domain) and the active VL domain. Upon proteolytic cleavage by EK, the VLi domain of Pro16 and the VHi domain of Pro19 are released from their respective prodrug constructs, leaving two molecules that associate on the surface of an EGFR-expressing cell due to innate self-assembly of the anti-CD3 variable heavy and variable light domains, each having an antigen binding domain to a tumor antigen as well, thus allowing the recruitment of T cells to the tumor site.

### II. Definitions

In order that the application may be more completely understood, several definitions are set forth below. Such definitions are meant to encompass grammatical equivalents.

The term “COBRA™” and “conditional bispecific redirected activation” refers to a bispecific conditionally effective protein that has a number of functional protein domains. In some embodiments, one of the functional domain is an antigen binding domain (ABD) that binds a target tumor antigen (TTA). In certain embodiments, another domain is an ABD that binds to a T cell antigen under certain conditions. The T cell antigen includes but is not limited to CD3. The term “hemi-COBRA™” refers to a conditionally effective protein that can bind a T cell antigen when a variable heavy chain of a hemi-COBRA can associate to a variable light chain of another hemi-COBRA™ (a complementary hemi-COBRA™) due to innate self-assembly when concentrated on the surface of a target expressing cell.

By “amino acid” and “amino acid identity” as used herein is meant one of the 20 naturally occurring amino acids or any non-natural analogues that may be present at a specific, defined position. In many embodiments, “amino acid” means one of the 20 naturally occurring amino acids. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs”, such as peptoids (see, e.g., Simon et al., PNAS USA 89(20):9367 (1992)), although in general, proteins comprising only naturally occurring amino acids are preferred. Thus “amino acid”, or “peptide residue”, as used herein means both naturally occurring and synthetic amino acids. For example, homophenylalanine, citrulline and noreleucine are considered amino acids for the purposes of the invention. “Amino acid” also includes imino acid residues such as proline and hydroxyproline. The side chain may be in either the (R) or the (S) configuration. In the preferred embodiment, the amino acids are in the (S) or L-configuration. If non-naturally occurring side chains are used, non-amino acid substituents may be used, for example to prevent or retard in vivo degradation.

By “amino acid modification” herein is meant an amino acid substitution, insertion, and/or deletion in a polypeptide sequence or an alteration to a moiety chemically linked to a protein. For example, a modification may be an altered carbohydrate or PEG structure attached to a protein. For clarity, unless otherwise noted, the amino acid modification is always to an amino acid coded for by DNA, e.g., the 20 amino acids that have codons in DNA and RNA. The preferred amino acid modification herein is a substitution.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For clarity, a protein which has been engineered to change the nucleic acid coding sequence but not change the starting amino acid (for example, exchanging CGG (encoding arginine) to CGA (still encoding arginine) to increase host organism expression levels) is not an “amino acid substitution”; that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence.

By “amino acid deletion” or “deletion” as used herein is meant the removal of an amino acid sequence at a particular position in a parent polypeptide sequence.

The polypeptides of the invention specifically bind to CD3 and target tumor antigens (TTAs) such as target cell receptors, as outlined herein. “Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore assay or Octet as is known in the art.

By “parent polypeptide” or “precursor polypeptide” (including Fc parent or precursors) as used herein is meant a polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it. Accordingly, by “parent Fc polypeptide” as used herein is meant an unmodified Fc polypeptide that is modified to generate a variant, and by “parent antibody” as used herein is meant an unmodified antibody that is modified to generate a variant antibody.

By “position” as used herein is meant a location in the sequence of a protein. Positions may be numbered sequentially, or according to an established format, for example the EU index for antibody numbering.

By “target antigen” as used herein is meant the molecule that is bound specifically by the variable region of a given antibody. A target antigen may be a protein, carbohydrate, lipid, or other chemical compound. A range of suitable exemplary target antigens are described herein.

By “target cell” as used herein is meant a cell that expresses a target antigen.

By “Fv” or “Fv domain” or “Fv region” as used herein is meant a polypeptide that comprises the VL and VH domains of an antibody, generally a human IgG1 antibody. Fv domains usually form an “antigen binding domain” or “ABD” as discussed herein, if they contain active VH and VL domains and do not have a constrained linker. As discussed below, Fv domains can be organized in a number of ways in the present invention, and can be “active” or “inactive”, such as in a scFv format, a constrained Fv format, a pseudo Fv format, etc.

By “variable domain” herein is meant the region of an immunoglobulin that comprises one or more Ig domains substantially encoded by any of the V.kappa., V.lamda., and/or VH genes that make up the kappa, lambda, and heavy chain immunoglobulin genetic loci respectively.

Each VH and VL is composed of three hypervariable regions (“complementary determining regions,” “CDRs”) and four “framework regions”, or “FRs”, arranged from amino-terminus to carboxy-terminus in the following order: FR1-CDR1-FR2-CDR2-FR3-CDR3-FR4. Thus, the VH domain has the structure vhFR1-vhCDR1-vhFR2-vhCDR2-vhFR3-vhCDR3-vhFR4 and the VL domain has the structure vlFR1-vlCDR1-vlFR2-vlCDR2-vlFR3-vlCDR3-vlFR4. As is more fully described herein, the vhFR regions and the vlFR regions self assemble to form Fv domains. In general, in the prodrug formats of the invention, there are “pseudo Fv domains” for which the CDRs do not form antigen binding domains when self associated.

The hypervariable regions confer antigen binding specificity and generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk (1987) J. Mol. Biol. 196:901-917. Specific CDRs of the invention are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g. vhCDR1, vhCDR2 and vhCDR3) and the disclosure of each variable light region is a disclosure of the vhCDRs (e.g. vlCDR1, vlCDR2 and vlCDR3).

A useful comparison of CDR numbering is as below, see Lafranc et al., Dev. Comp. Immunol. 27(1):55-77 (2003):

TABLE 1

| | Kabat + | | | | | |
|---|---|---|---|---|---|---|
| | Chothia | IMGT | Kabat | AbM | Chothia | Contact |
| vhCDR1 | 26-35 | 27-38 | 31-35 | 26-35 | 26-32 | 30-35 |
| vhCDR2 | 50-65 | 56-65 | 50-65 | 50-58 | 52-56 | 47-58 |
| vhCDR3 | 95-102 | 105-117 | 95-102 | 95-102 | 95-102 | 93-101 |
| vlCDR1 | 24-34 | 27-38 | 24-34 | 24-34 | 24-34 | 30-36 |
| vlCDR2 | 50-56 | 56-65 | 50-56 | 50-56 | 50-56 | 46-55 |
| vlCDR3 | 89-97 | 105-117 | 89-97 | 89-97 | 89-97 | 89-96 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present invention provides a large number of different CDR sets. In this case, a “full CDR set” comprises the three variable light and three variable heavy CDRs, e.g. a vlCDR1, vlCDR2, vlCDR3, vhCDR1, vhCDR2 and vhCDR3. As will be appreciated by those in the art, each set of CDRs, the VH and VL CDRs, can bind to antigens, both individually and as a set. For example, in constrained Fv domains, the vhCDRs can bind, for example to CD3 and the vhCDRs can bind to CD3, but in the constrained format they cannot bind to CD3.

These CDRs can be part of a larger variable light or variable heavy domain, respectfully. In addition, as more fully outlined herein, the variable heavy and variable light domains can be on separate polypeptide chains or on a single polypeptide chain in the case of scFv sequences.

The CDRs contribute to the formation of the antigen-binding, or more specifically, epitope binding sites. “Epitope” refers to a determinant that interacts with a specific antigen binding site in the variable regions known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

The variable heavy and variable light domains of the invention can be “active” or “inactive”.

As used herein, “inactive VH” (“VHi”) and “inactive VL” (“VLi”) refer to components of a pseudo Fv domain, which, when paired with their cognate VL or VH partners, respectively, form a resulting VH/VL pair that does not specifically bind to the antigen to which the “active” VH or “active” VL would bind were it bound to an analogous VL or VH, which was not “inactive”. Exemplary “inactive VH” and “inactive VL” domains are formed by mutation of a wild type VH or VL sequence. Exemplary mutations are within CDR1, CDR2 or CDR3 of VH or VL. An exemplary mutation includes placing a domain linker within CDR2, thereby forming an “inactive VH” or “inactive VL” domain. In contrast, an “active VH” or “active VL” is one that, upon pairing with its “active” cognate partner, i.e., VL or VH, respectively, is capable of specifically binding to its target antigen.

In contrast, as used herein, the term “active” refers to a CD3 binding domain that is capable of specifically binding to CD3. This term is used in two contexts: (a) when referring to a single member of an Fv binding pair (i.e., VH or VL), which is of a sequence capable of pairing with its cognate partner and specifically binding to CD3; and (b) the pair of cognates (i.e., VH and VL) of a sequence capable of specifically binding to CD3. An exemplary “active” VH, VL or VH/VL pair is a wild type or parent sequence.

“CD-x” refers to a cluster of differentiation (CD) protein. In exemplary embodiments, CD-x is selected from those CD proteins having a role in the recruitment or activation of T-cells in a subject to whom a polypeptide construct of the invention has been administered. In an exemplary embodiment, CD-x is CD3.

The term “binding domain” characterizes, in connection with the present invention, a domain which (specifically) binds to/interacts with/recognizes a given target epitope or a given target site on the target molecules (antigens), for example: EGFR and CD3, respectively. The structure and function of the target antigen binding domain (recognizing EGFR), and preferably also the structure and/or function of the CD3 binding domain (recognizing CD3), is/are based on the structure and/or function of an antibody, e.g. of a full-length or whole immunoglobulin molecule, including sdAbs. According to the invention, the target antigen binding domain is generally characterized by the presence of three CDRs that bind the target tumor antigen (generally referred to in the art as variable heavy domains, although no corresponding light chain CDRs are present). Alternatively, ABDs to TTAs can include three light chain CDRs (i.e., CDR1, CDR2 and CDR3 of the VL region) and/or three heavy chain CDRs (i.e. CDR1, CDR2 and CDR3 of the VH region). The CD3 binding domain preferably also comprises at least the minimum structural requirements of an antibody which allow for the target binding. More preferably, the CD3 binding domain comprises at least three light chain CDRs (i.e. CDR1, CDR2 and CDR3 of the VL region) and/or three heavy chain CDRs (i.e. CDR1, CDR2 and CDR3 of the VH region). It is envisaged that in exemplary embodiments the target antigen and/or CD3 binding domain is produced by or obtainable by phage-display or library screening methods.

By “domain” as used herein is meant a protein sequence with a function, as outlined herein. Domains of the invention include tumor target antigen binding domains (TTA domains), variable heavy domains, variable light domains, linker domains, and half life extension domains.

By “domain linker” (“DL”) herein is meant an amino acid sequence that joins two domains as outlined herein. Domain linkers can be cleavable linkers, constrained cleavable linkers, non-cleavable linkers, constrained non-cleavable linkers, scFv linkers, etc.

By “cleavable linker” (“CL”) herein is meant an amino acid sequence that can be cleaved by a protease, preferably a human protease in a disease tissue as outlined herein. Cleavable linkers generally are at least 3 amino acids in length, with from 4, 5, 6, 7, 8, 9, 10, 11, 12, 13, 14, 15 or more amino acids finding use in the invention, depending on the required flexibility.

By “non cleavable linker” (“NCL”) herein is meant an amino acid sequence that cannot be cleaved by a human protease under normal physiological conditions.

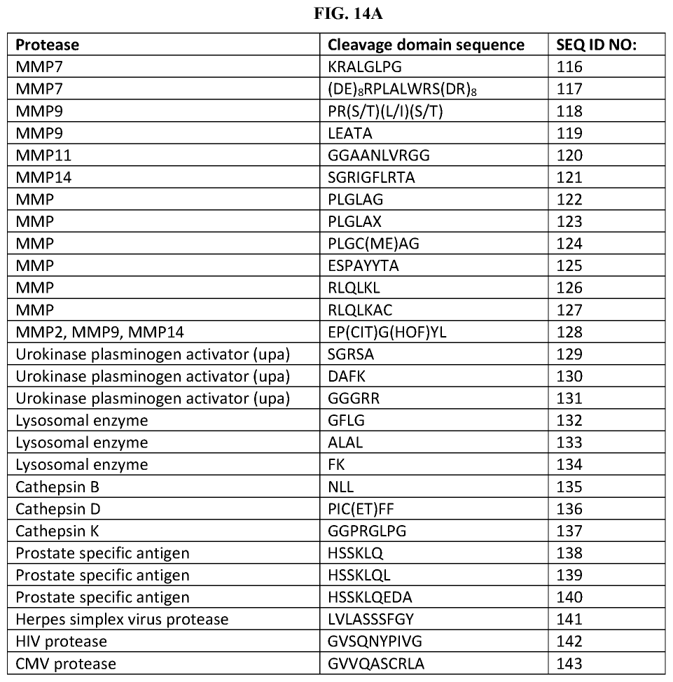

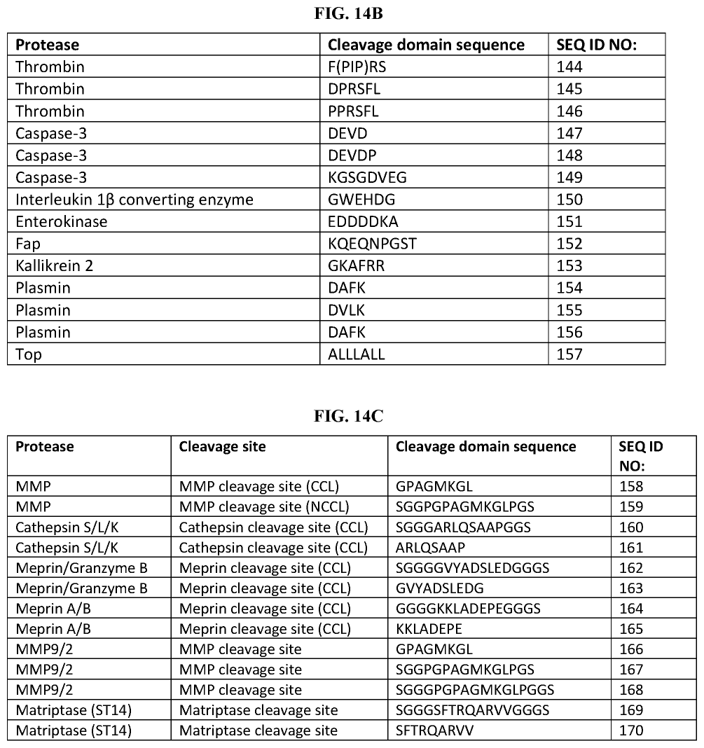

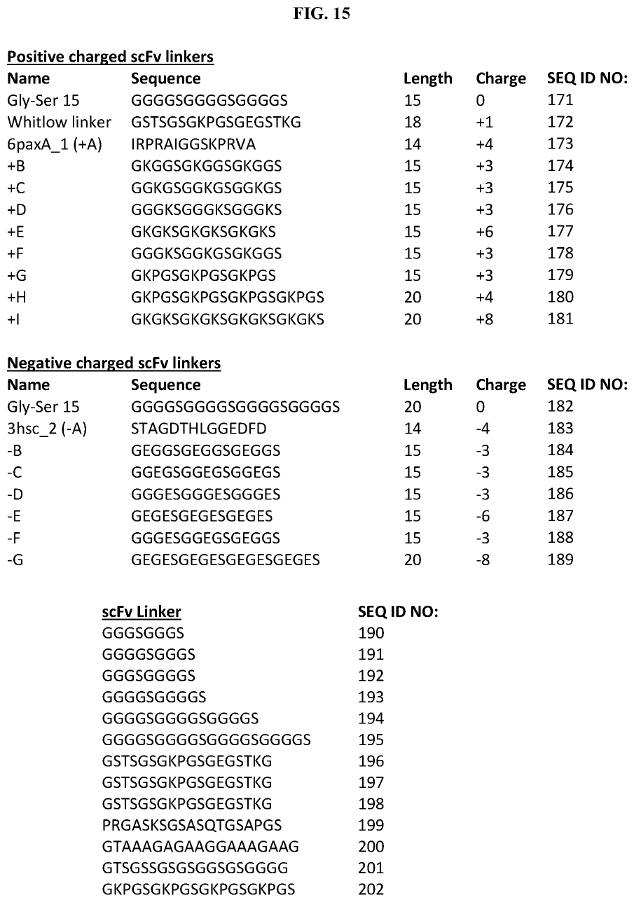

By “constrained cleavable linker” (“CCL”) herein is meant a short polypeptide that contains a protease cleavage site (as defined herein) that joins two domains as outlined herein in such a manner that the two domains cannot significantly interact with each other until after they reside on different polypeptide chains, e.g., after cleavage. When the CCL joins a VH and a VL domain as defined herein, the VH and VL cannot self-assemble to form a functional Fv prior to cleavage due to steric constraints in an intramolecular way. Upon cleavage by the relevant protease, the VH and VL can assemble to form an active antigen binding domain in a intermolecular way. In general, CCLs are less than 10 amino acids in length, with 9, 8, 7, 6, 5 and 4 amino acids finding use in the invention. In general, protease cleavage sites generally are at least 4+ amino acids in length to confer sufficient specificity, as shown in FIG. 14A-FIG. 14C.

By “constrained non-cleavable linker” (“CNCL”) herein is meant a short polypeptide that that joins two domains as outlined herein in such a manner that the two domains cannot significantly interact with each other, and that is not significantly cleaved by human proteases under physiological conditions.

By “constrained Fv domain” herein is meant an Fv domain that comprises an active variable heavy domain and an active variable light domain, linked covalently with a constrained linker as outlined herein, in such a way that the active heavy and light variable domains cannot intramolecularly interact to form an active Fv that will bind an antigen such as CD3. Thus, a constrained Fv domain is one that is similar to an scFv but is not able to bind an antigen due to the presence of a constrained linker.

By “pseudo Fv domain” herein is meant a domain that comprises a pseudo or inactive variable heavy domain and a pseudo or inactive variable light domain, linked using a domain linker (which can be cleavable, constrained, non-cleavable, non-constrained, etc.). The VHi and VLi domains of a pseudo Fv domain do not bind to a human antigen when either associated with each other (VHi/VLi) or when associated with an active VH or VL; thus VHi/VLi, VHi/VL and VLi/VH Fv domains do not appreciably bind to a human protein, such that these domains are inert in the human body.

By “single chain Fv” or “scFv” herein is meant a variable heavy (VH) domain covalently attached to a variable light (VL) domain, generally using a scFv linker as discussed herein, to form a scFv or scFv domain. A scFv domain can be in either orientation from N- to C-terminus (VH-linker-VL or VL-linker-VH).

By “single domain Fv”, “sdFv”, “single domain antibody” or “sdAb” herein is meant an antigen binding domain that only has three CDRs, generally based on camelid antibody technology. See: Protein Engineering 9(7):1129-35 (1994); Rev Mol Biotech 74:277-302 (2001); Ann Rev Biochem 82:775-97 (2013).

By “protease cleavage site” refers to the amino acid sequence recognized and cleaved by a protease. Suitable protease cleavage sites are outlined below.

As used herein, “protease cleavage domain” refers to the peptide sequence incorporating the “protease cleavage site” and any linkers between individual protease cleavage sites and between the protease cleavage site(s) and the other functional components of the constructs of the invention (e.g., V_{H}, V_{L}, V_{Hi}, V_{Li}, target antigen binding domain(s), half-life extension domain, etc.).

By “Fc” or “Fc region” or “Fc domain” as used herein is meant the polypeptide comprising the constant region of an antibody excluding the first constant region immunoglobulin domain and in some cases, part of the hinge. Thus Fc refers to the last two constant region immunoglobulin domains of IgA, IgD, and IgG, the last three constant region immunoglobulin domains of IgE and IgM, and the flexible hinge N-terminal to these domains. For IgA and IgM, Fc may include the J chain. For IgG, the Fc domain comprises immunoglobulin domains Cγ2 and Cγ3 (Cγ2 and Cγ3) and the lower hinge region between Cγ1 (Cγ1) and Cγ2 (Cγ2). Although the boundaries of the Fc region may vary, the human IgG heavy chain Fc region is usually defined to include residues C226 or P230 to its carboxyl-terminus, wherein the numbering is according to the EU index as in Kabat.

By “substantially equivalent” in the context of an amount, ratio, level, and the like refers to a near equal or equal quantity. In some embodiments, a substantially equivalent amount refers to a 10% difference or less, e.g., a 10%, 9%, 8%, 7%, 6%, 5%, 4%, 3%, 2%, 1% or less difference, between two amounts. In some embodiments, a substantially equivalent amount refers two equal or same amounts, ratios, levels, or quantities. In some cases, a substantially equivalent amount of polypeptides refers to an equal amount of a first polypeptide and a second polypeptide. In some cases, a substantially equivalent ratio of polynucleotides refers to an equal amount of a first polynucleotide and a second polynucleotide.

### III. Description of Embodiments

A. Polypeptide Pairs and Prodrug Constructs

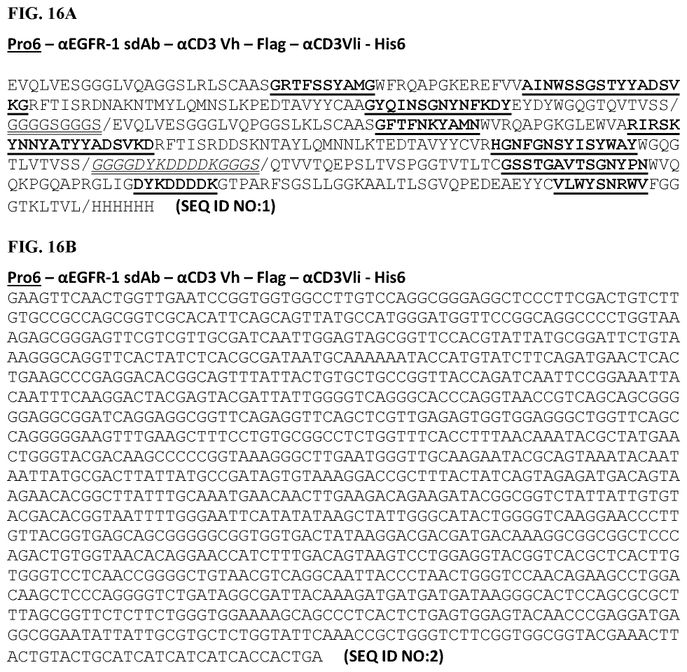

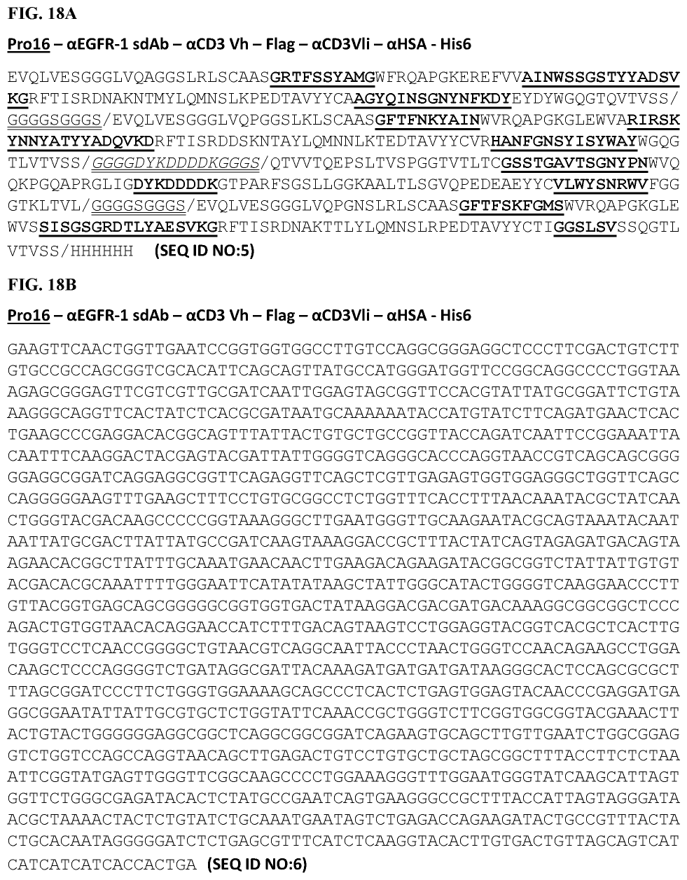

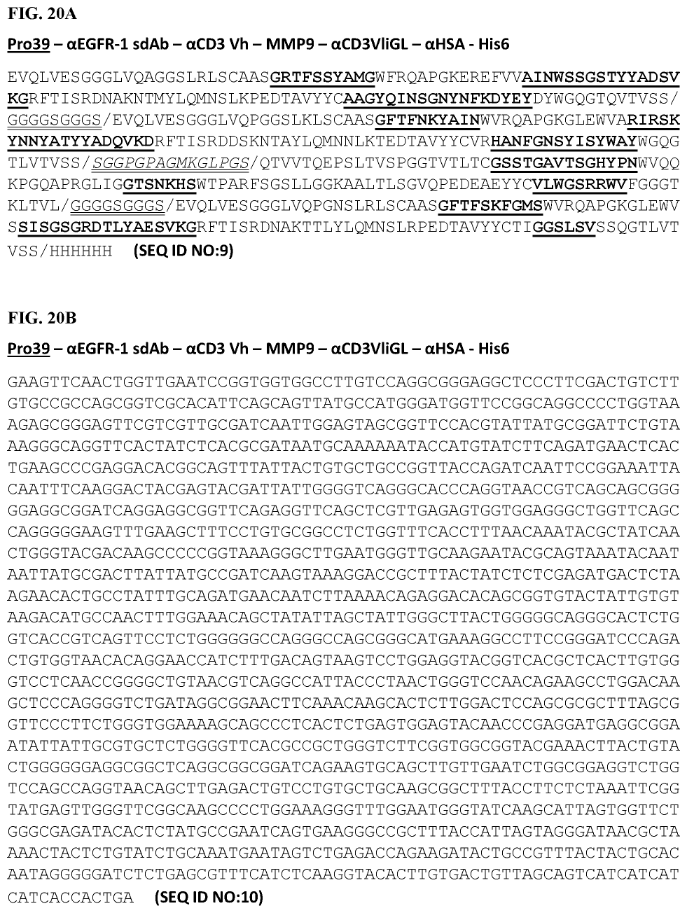

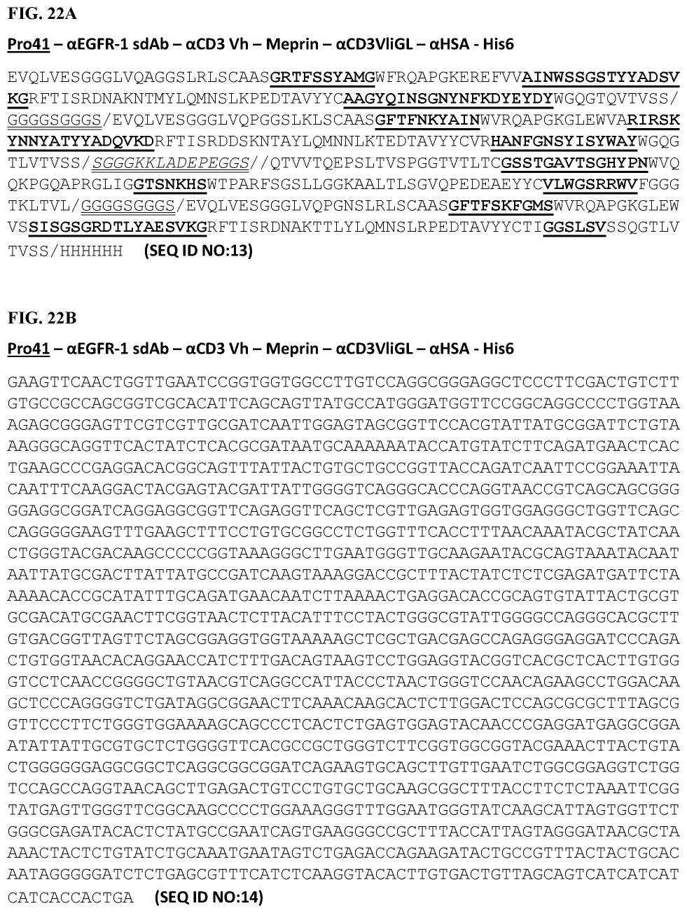

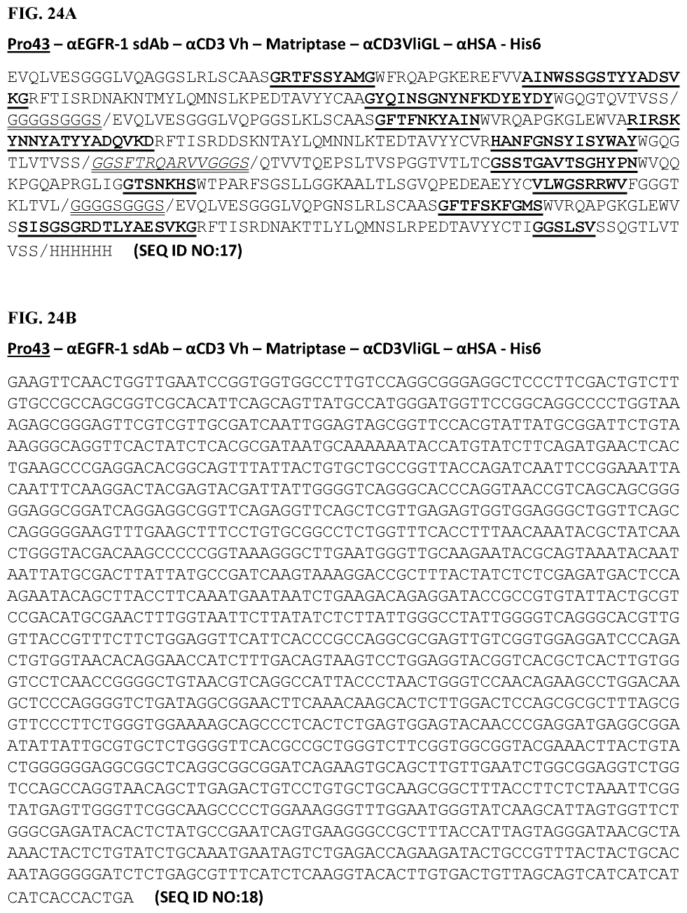

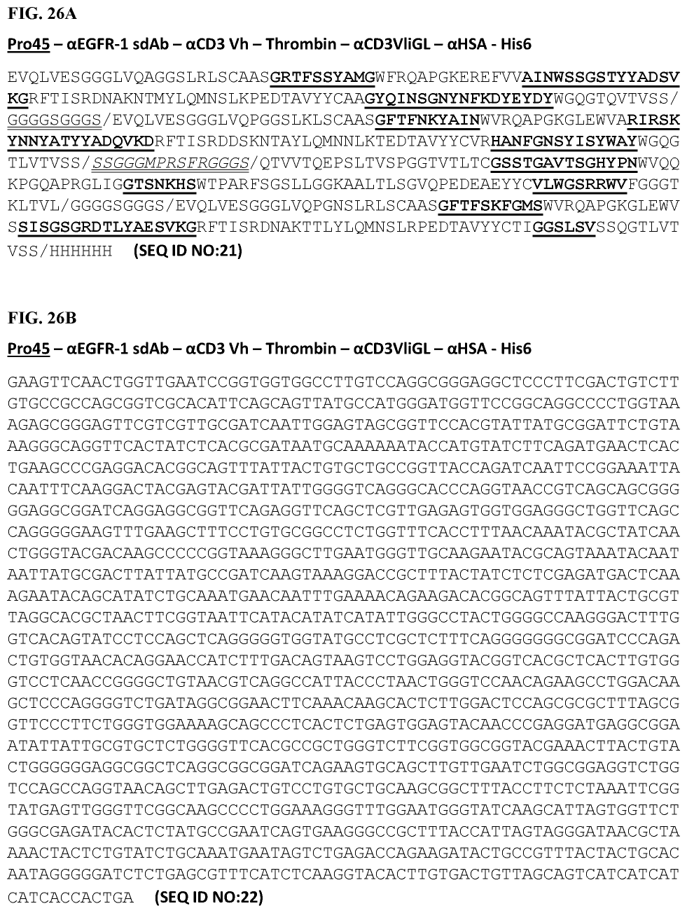

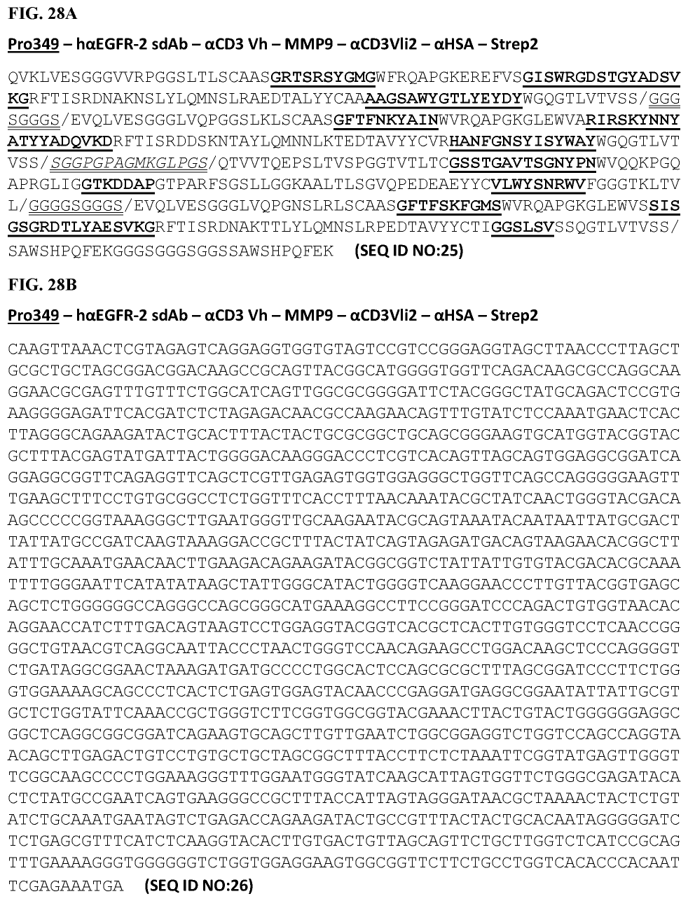

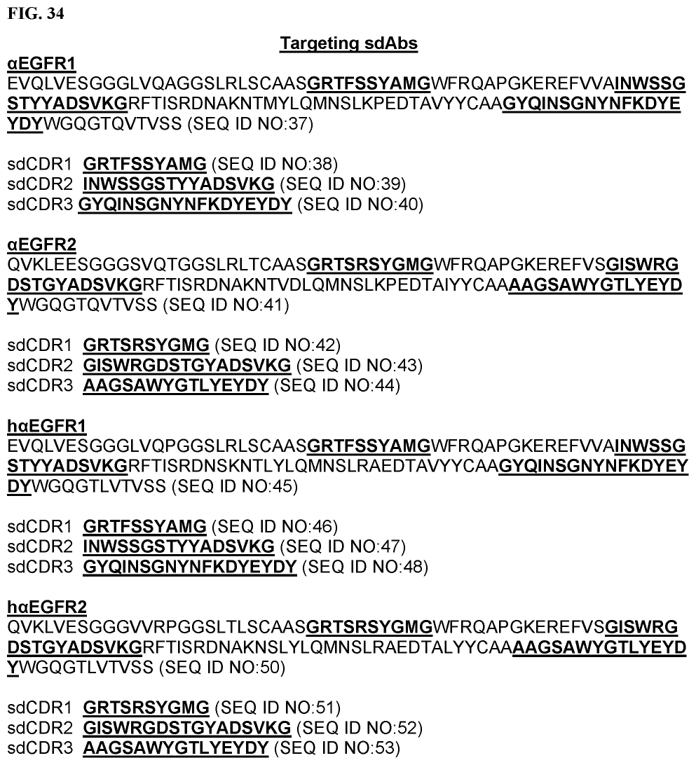

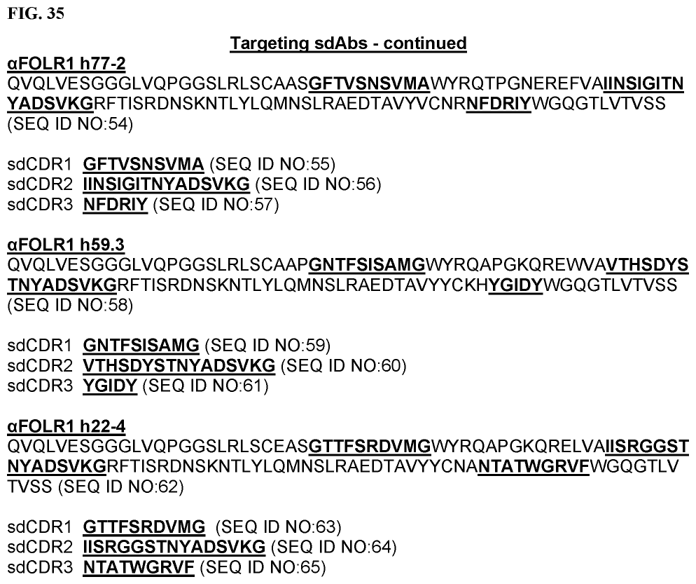

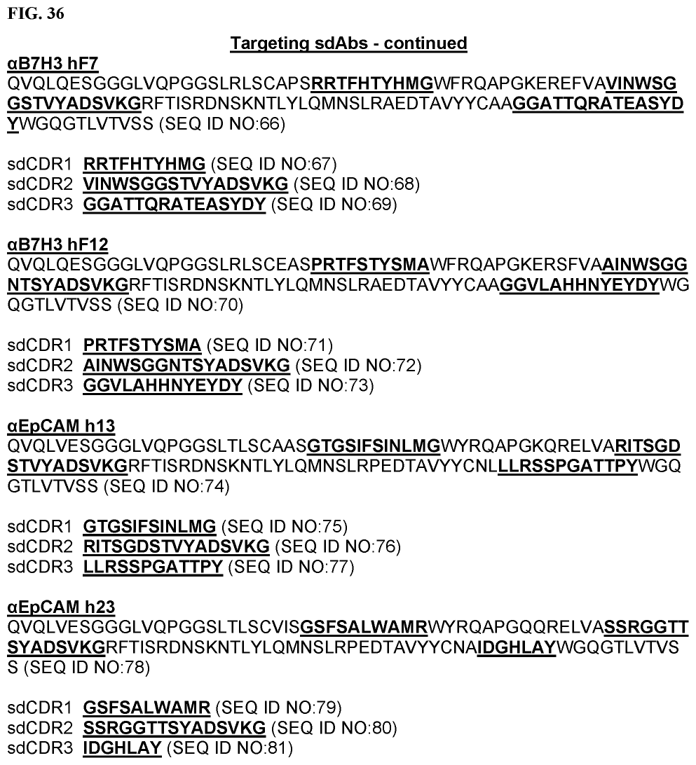

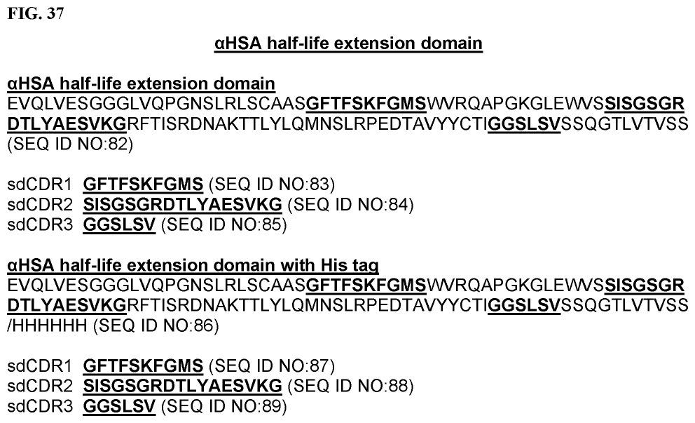

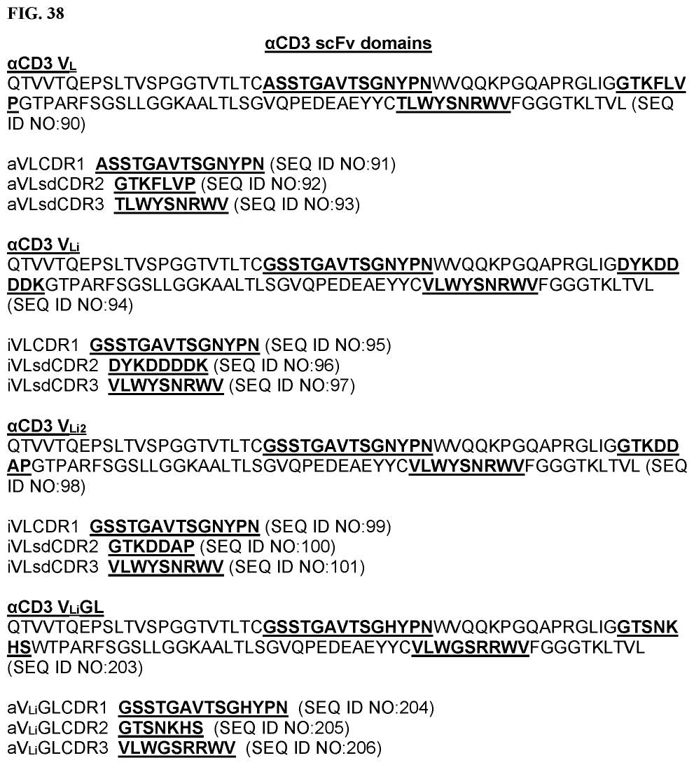

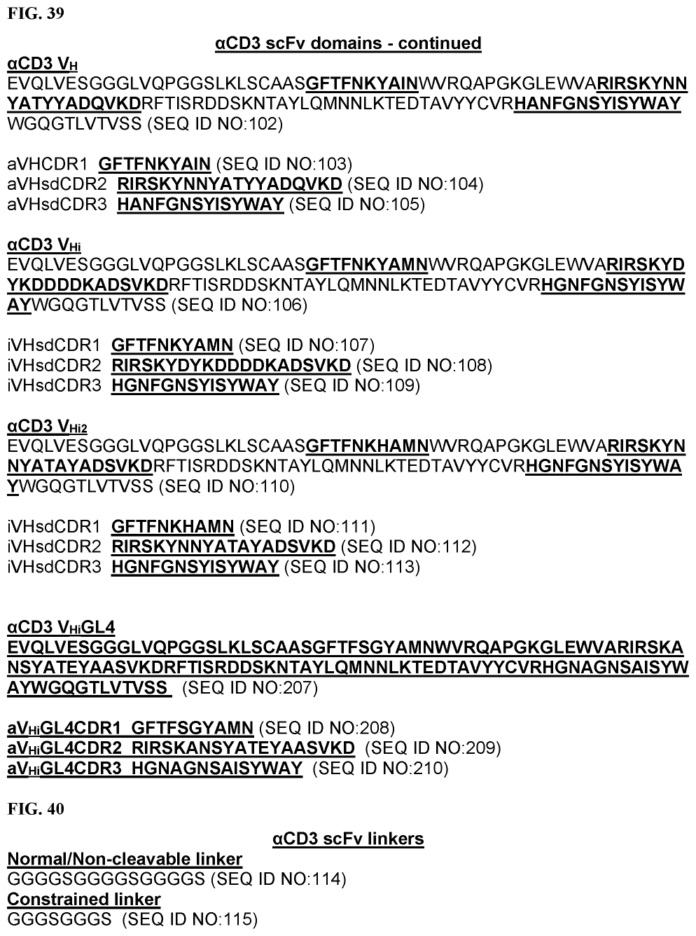

In some embodiments, the polypeptide has the structure (N-terminus to C-terminus): sdAb(TTA)-DL-hFR1-vhCDR1-vhFR2-vhCDR2-vhFR3-vhCDR3-vhFR4-CL-vlFR1-vliCDR1-vlFR2-vliCDR2-vlFR3-vliCDR3-vlFR4. In some embodiments, the CDRs and/or variable domains (e.g., active heavy chain domain or pseudo light chain) are any of those of depicted in FIG. 16A, FIG. 18A, FIG. 20A, FIG. 22A, FIG. 24A, FIG. 26A, FIG. 28A, FIG. 38, and FIG. 39. In some embodiments, the sdAb(TTA) of the polypeptide are any one of those depicted in FIG. 34, FIG. 35, and FIG. 36. In certain embodiments, the polypeptide has the structure of any one selected from the group consisting of Pro6 (SEQ ID NO:2), Pro16 (SEQ ID NO:5), Pro39 (SEQ ID NO:9), Pro41 (SEQ ID NO:13), Pro43 (SEQ ID NO:17), Pro45 (SEQ ID NO:21), and Pro349 (SEQ ID NO:25).

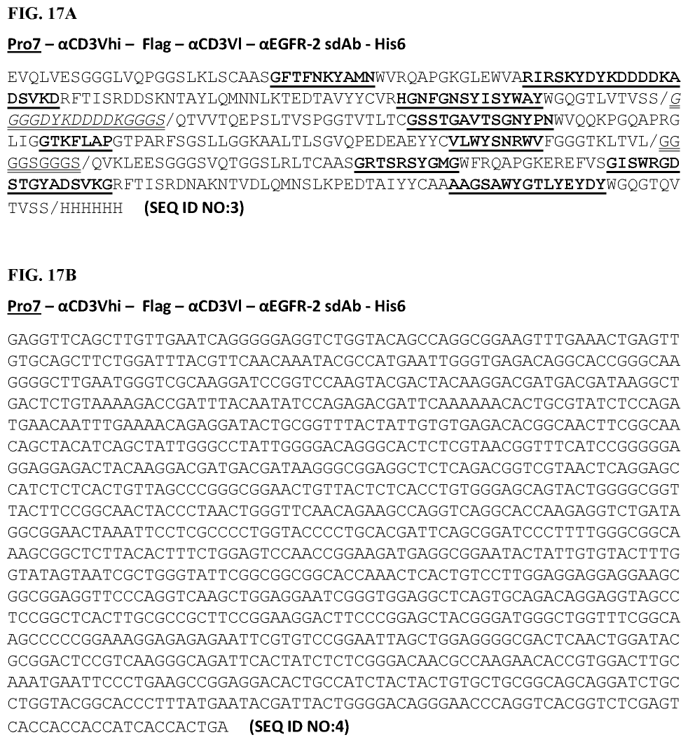

In one embodiment, the polypeptide has the structure (N-terminus to C-terminus): vhFR1-vhiCDR1-vhFR2-vhiCDR2-vhFR3-vhiCDR3-vhFR4-CL-vlFR1-vlCDR1-vlFR2-vlCDR2-vlFR3-vlCDR3-vlFR4-DL-sdAb(TTA). In some embodiments, the CDRs and/or variable domains (e.g., pseudo heavy chain domain or active light chain) are those of FIG. 17A, FIG. 38, and FIG. 39. In some embodiments, the sdAb(TTA) of the polypeptide are any one of those depicted in FIG. 34, FIG. 35, and FIG. 36. In certain embodiments, the polypeptide has the structure of Pro7 (SEQ ID NO:3).

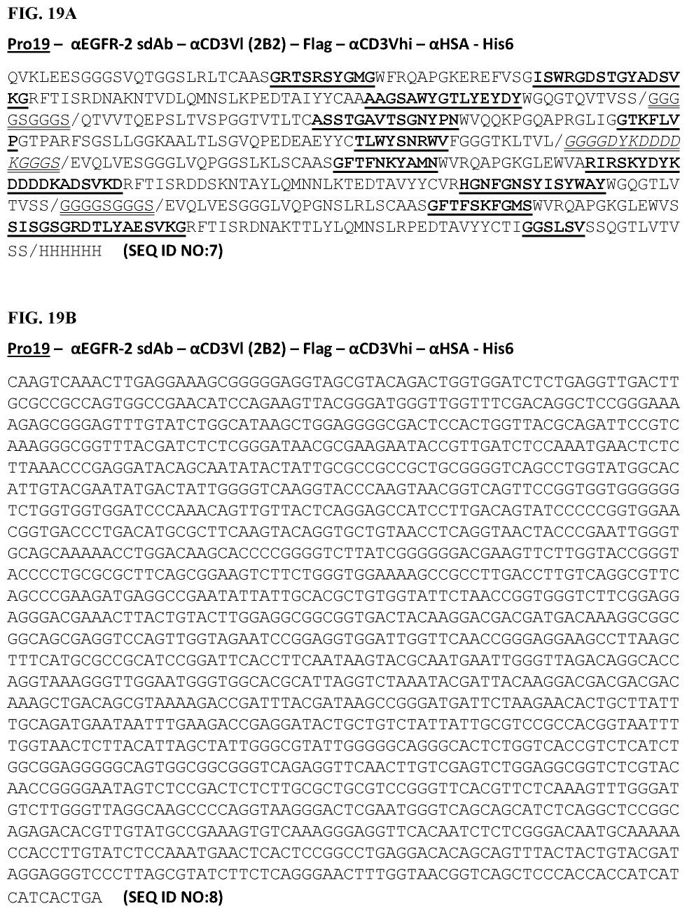

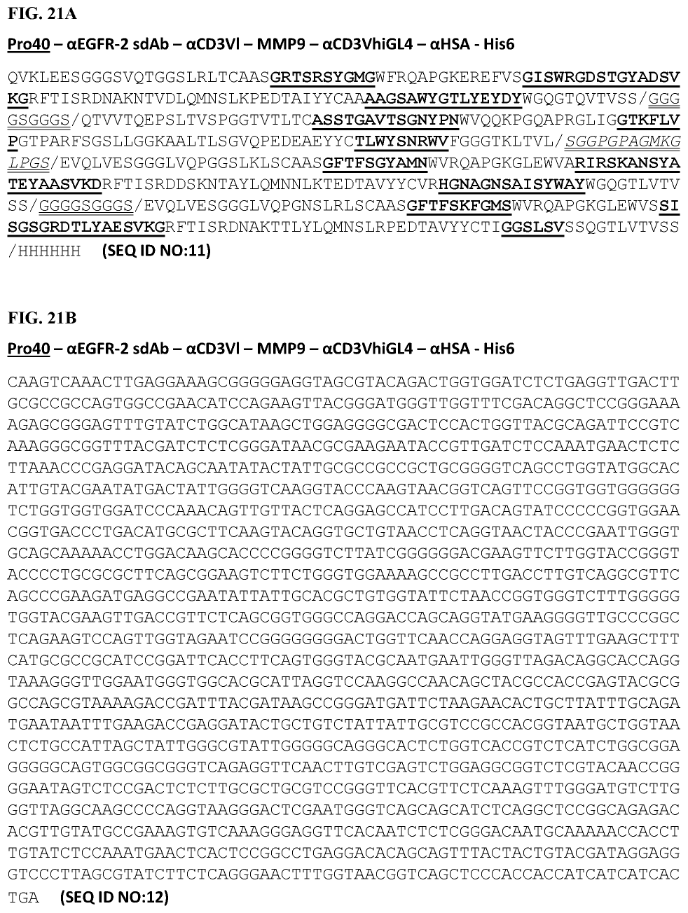

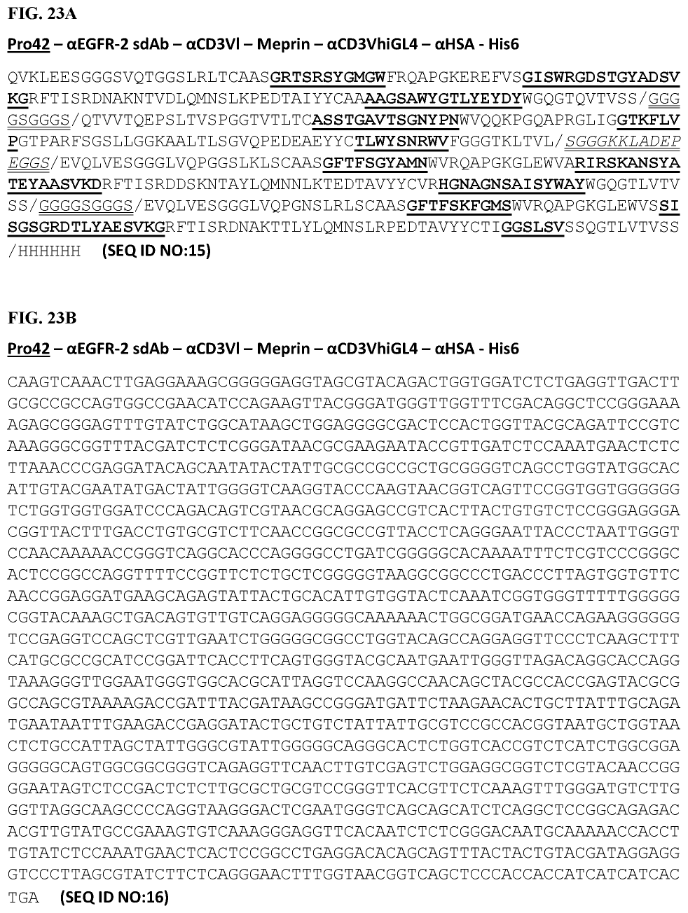

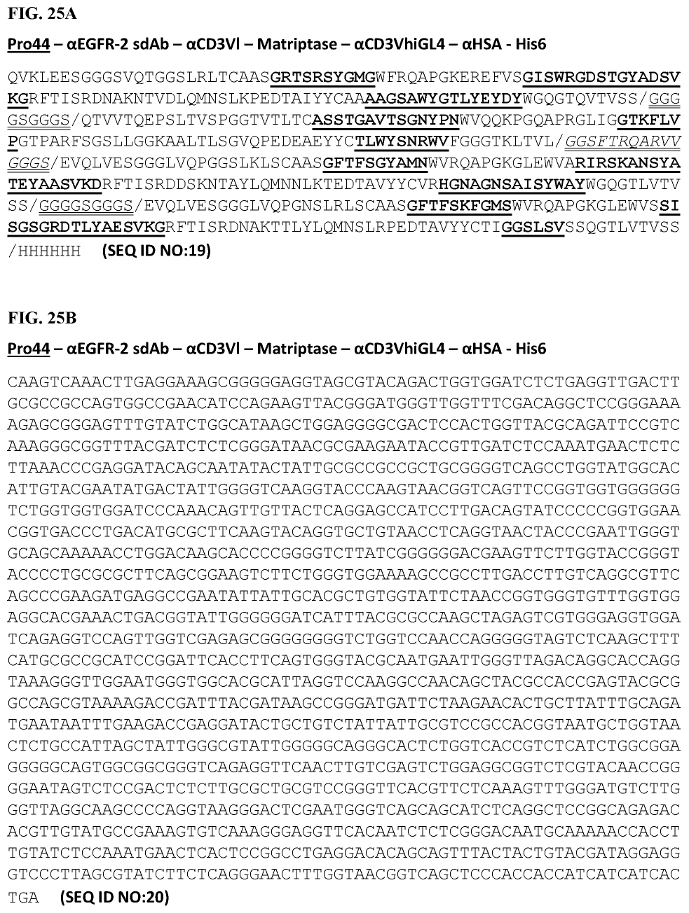

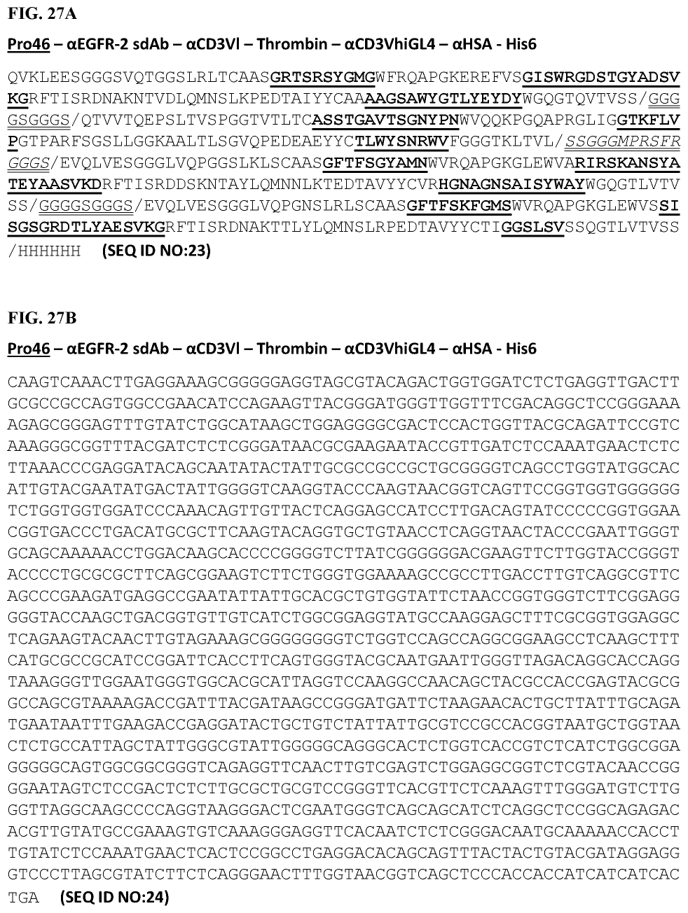

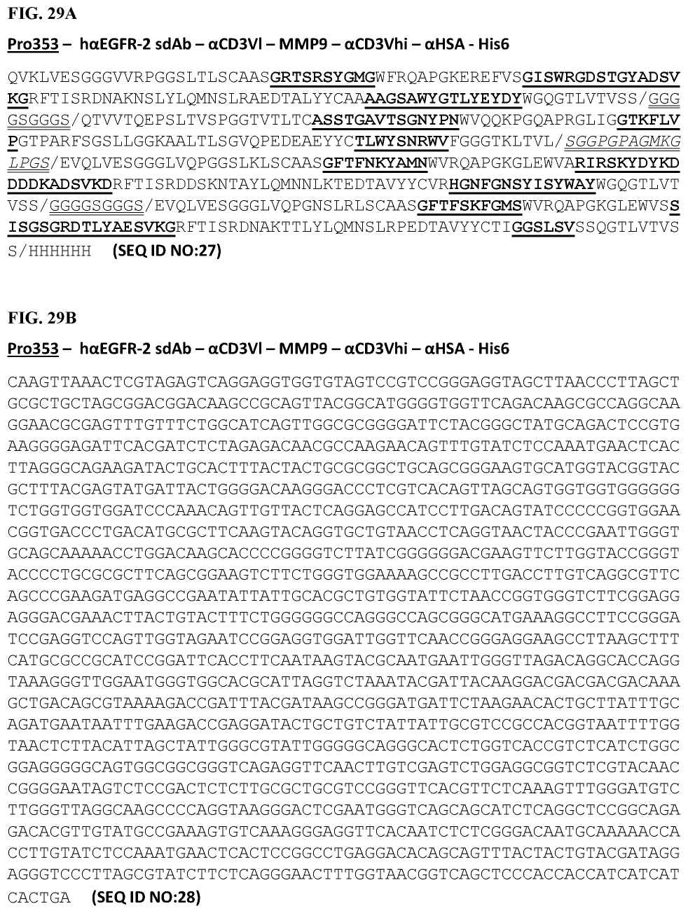

In various embodiments, the polypeptide has the structure (N-terminus to C-terminus): sdAb(TTA)-DL-vlFR1-vlCDR1-vlFR2-vlCDR2-vlFR3-vlCDR3-vlFR4-CL-vhFR1-vhiCDR1-vhFR2-vhiCDR2-vhFR3-vhiCDR3-vhFR4. In some embodiments, the CDRs and/or variable domains (e.g., pseudo heavy chain domain or active light chain) are any of those of depicted in FIG. 19A, FIG. 21A, FIG. 23A, FIG. 25A, FIG. 27A, FIG. 29A, FIG. 38, and FIG. 39. In some embodiments, the sdAb(TTA) of the polypeptide are any one of those depicted in FIG. 34, FIG. 35, and FIG. 36. In certain embodiments, the polypeptide has the structure of any one selected from the group consisting of Pro19 (SEQ ID NO:7), Pro40 (SEQ ID NO:11), Pro42 (SEQ ID NO:15), Pro44 (SEQ ID NO:19), Pro46 (SEQ ID NO:23), and Pro353 (SEQ ID NO:27).

In one embodiment, provided herein is a prodrug composition (or prodrug construct) comprises a first polypeptide comprising a structure of sdAb(TTA)-DL-vhFR1-vhCDR1-vhFR2-vhCDR2-vhFR3-vhCDR3-vhFR4-CL-vlFR1-vliCDR1-vlFR2-vliCDR2-vlFR3-vliCDR3-vlFR4 and a second polypeptide comprising a structure of vhFR1-vhiCDR1-vhFR2-vhiCDR2-vhFR3-vhiCDR3-vhFR4-CL-vlFR1-vlCDR1-vlFR2-vlCDR2-vlFR3-vlCDR3-vlFR4-DL-sdAb(TTA). In some embodiments, the prodrug composition (or prodrug construct) comprises a first polypeptide comprising a structure shown in FIG. 16A, and a second polypeptide comprising a structure shown in FIG. 17A. In certain embodiments, the prodrug composition (or prodrug construct) comprises Pro6 and Pro7. In some instances, the sdAb of the first polypeptide binds to the same antigen as the sdAb of the second polypeptide. In other instances, the sdAb of the first polypeptide binds to the same epitope of the antigen as the sdAb of the second polypeptide. In yet other instances, the sdAb of the first polypeptide binds a different antigen than the sdAb of the second polypeptide.

In some embodiments, provided herein is a prodrug composition (or prodrug construct) comprises a first polypeptide comprising a structure of sdAb(TTA)-DL-vhFR1-vhCDR1-vhFR2-vhCDR2-vhFR3-vhCDR3-vhFR4-CL-vlFR1-vliCDR1-vlFR2-vliCDR2-vlFR3-vliCDR3-vlFR4 and a second polypeptide comprising a structure of sdAb(TTA)-DL-vlFR1-vlCDR1-vlFR2-vlCDR2-vlFR3-vlCDR3-vlFR4-CL-vhFR1-vhiCDR1-vhFR2-vhiCDR2-vhFR3-vhiCDR3-vhFR4. In other instances, the sdAb of the first polypeptide binds to the same epitope of the antigen as the sdAb of the second polypeptide. In yet other instances, the sdAb of the first polypeptide binds a different antigen than the sdAb of the second polypeptide.

In some embodiments, the prodrug composition (or prodrug construct) comprises a first polypeptide comprising a structure shown in FIG. 18A and a second polypeptide comprising a structure shown in FIG. 19A. In some embodiments, the prodrug composition (or prodrug construct) comprises Pro16 and Pro19.

In some embodiments, the prodrug composition (or prodrug construct) comprises a first polypeptide comprising a structure shown in FIG. 20A and a second polypeptide comprising a structure shown in FIG. 21A. In certain embodiments, the prodrug composition (or prodrug construct) comprises Pro39 and Pro40.

In some embodiments, the prodrug composition (or prodrug construct) comprises a first polypeptide comprising a structure shown in FIG. 22A and a second polypeptide comprising a structure shown in FIG. 23A. In particular embodiments, the prodrug composition (or prodrug construct) comprises Pro41 and Pro42.

In some embodiments, the prodrug composition (or prodrug construct) comprises a first polypeptide comprising a structure shown in FIG. 24A and a second polypeptide comprising a structure shown in FIG. 25A. In some embodiments, the prodrug composition (or prodrug construct) comprises Pro43 and Pro44.

In some embodiments, the prodrug composition (or prodrug construct) comprises a first polypeptide comprising a structure shown in FIG. 26A and a second polypeptide comprising a structure shown in FIG. 27A. In some embodiments, the prodrug composition (or prodrug construct) comprises Pro45 and Pro46.

In some embodiments, the prodrug composition (or prodrug construct) comprises a first polypeptide comprising a structure shown in FIG. 28A and a second polypeptide comprising a structure shown in FIG. 29A. In certain embodiments, the prodrug composition (or prodrug construct) comprises Pro349 and Pro353.

B. Expression Vectors and Host Cells

In one embodiment, provided herein is an expression vector comprising a first polynucleotide encoding a first polypeptide comprising a structure of sdAb(TTA)-DL-hFR1-vhCDR1-vhFR2-vhCDR2-vhFR3-vhCDR3-vhFR4-CL-vlFR1-vliCDR1-vlFR2-vliCDR2-vlFR3-vliCDR3-vlFR4, and/or a second polynucleotide encoding a second polypeptide comprising a structure of vhFR1-vhiCDR1-vhFR2-vhiCDR2-vhFR3-vhiCDR3-vhFR4-CL-vlFR1-vlCDR1-vlFR2-vlCDR2-vlFR3-vlCDR3-vlFR4-DL-sdAb(TTA). In some cases, the sdAb of the first polypeptide binds to the same antigen (TTA) as the sdAb of the second polypeptide. In other instances, the sdAb of the first polypeptide binds to the same epitope of the antigen as the sdAb of the second polypeptide. In yet other instances, the sdAb of the first polypeptide binds a different antigen than the sdAb of the second polypeptide.

In another embodiment, provided herein is a host cell comprising a first polynucleotide encoding a first polypeptide comprising a structure of sdAb(TTA)-DL-hFR1-vhCDR1-vhFR2-vhCDR2-vhFR3-vhCDR3-vhFR4-CL-vlFR1-vliCDR1-vlFR2-vliCDR2-vlFR3-vliCDR3-vlFR4, and/or a second polynucleotide encoding a second polypeptide comprising a structure of vhFR1-vhiCDR1-vhFR2-vhiCDR2-vhFR3-vhiCDR3-vhFR4-CL-vlFR1-vlCDR1-vlFR2-vlCDR2-vlFR3-vlCDR3-vlFR4-DL-sdAb(TTA). In some cases, the sdAb of the first polypeptide binds to the same antigen as the sdAb of the second polypeptide. In other instances, the sdAb of the first polypeptide binds to the same epitope of the antigen as the sdAb of the second polypeptide. In yet other instances, the sdAb of the first polypeptide binds a different antigen than the sdAb of the second polypeptide. In one embodiment, provided herein is a cell that expresses (produces or secretes) a first polypeptide comprising a structure of sdAb(TTA)-DL-hFR1-vhCDR1-vhFR2-vhCDR2-vhFR3-vhCDR3-vhFR4-CL-vlFR1-vliCDR1-vlFR2-vliCDR2-vlFR3-vliCDR3-vlFR4 and a second polypeptide comprising a structure of vhFR1-vhiCDR1-vhFR2-vhiCDR2-vhFR3-vhiCDR3-vhFR4-CL-vlFR1-vlCDR1-vlFR2-vlCDR2-vlFR3-vlCDR3-vlFR4-DL-sdAb(TTA).

In some embodiments, the host cell comprises an expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:1, as shown in FIG. 16A and/or a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:3, as shown in FIG. 17A. In some embodiments, the host cell comprises an expression vector comprising a first polynucleotide encoding a first polypeptide comprising a structure shown in FIG. 16A and/or a second polynucleotide encoding a second polypeptide comprising a structure shown in FIG. 17A. In certain embodiments, the expression vector comprises a first nucleic acid sequence shown in FIG. 16B and/or a second nucleic acid sequence shown in FIG. 17B. In some embodiments, the expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:2 and/or a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:4. In some embodiments, the expression vector comprises a nucleic acid sequence encoding Pro6 (SEQ ID NO: 2) and/or a nucleic acid sequence encoding Pro7 (SEQ ID NO: 4).

In some embodiments of the invention, a host cell comprises a first expression vector comprises a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:1, as shown in FIG. 16A and a second expression vector comprises a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:3, as shown in FIG. 17A. In some cases, the host cell comprises a first expression vector comprising a first nucleic acid sequence shown in FIG. 16B and a second expression vector comprising a second nucleic acid sequence shown in FIG. 17B. In some instances, the host cell comprises a first expression vector comprising a nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:2 and a second expression vector comprising a nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:4. In some instances, the host cell comprises a first expression vector comprising a nucleic acid sequence encoding Pro6 such as SEQ ID NO: 2 and a second expression vector comprising a nucleic acid sequence encoding Pro7 such as SEQ ID NO: 4.

In some embodiments, provided herein is an expression vector comprising a first polynucleotide encoding a first polypeptide comprising a structure of sdAb(TTA)-DL-hFR1-vhCDR1-vhFR2-vhCDR2-vhFR3-vhCDR3-vhFR4-CL-vlFR1-vliCDR1-vlFR2-vliCDR2-vlFR3-vliCDR3-vlFR4 and/or a second polynucleotide encoding a second polypeptide comprising a structure of sdAb(TTA)-DL-vlFR1-vlCDR1-vlFR2-vlCDR2-vlFR3-vlCDR3-vlFR4-CL-vhFR1-vhiCDR1-vhFR2-vhiCDR2-vhFR3-vhiCDR3-vhFR4. In some cases, the sdAb of the first polypeptide binds to the same antigen as the sdAb of the second polypeptide. In other instances, the sdAb of the first polypeptide binds to the same epitope of the antigen as the sdAb of the second polypeptide. In some instances, the sdAb of the first polypeptide binds to a different epitope of the antigen as the sdAb of the second polypeptide. In yet other instances, the sdAb of the first polypeptide binds a different antigen than the sdAb of the second polypeptide.

In some aspects, provided herein is a host cell comprising a first polynucleotide encoding a first polypeptide comprising a structure of sdAb(TTA)-DL-hFR1-vhCDR1-vhFR2-vhCDR2-vhFR3-vhCDR3-vhFR4-CL-vlFR1-vliCDR1-vlFR2-vliCDR2-vlFR3-vliCDR3-vlFR4 and/or a second polynucleotide encoding a second polypeptide comprising a structure of sdAb(TTA)-DL-vlFR1-vlCDR1-vlFR2-vlCDR2-vlFR3-vlCDR3-vlFR4-CL-vhFR1-vhiCDR1-vhFR2-vhiCDR2-vhFR3-vhiCDR3-vhFR4. In some cases, the sdAb of the first polypeptide binds to the same antigen as the sdAb of the second polypeptide. In other instances, the sdAb of the first polypeptide binds to the same epitope of the antigen as the sdAb of the second polypeptide. In some instances, the sdAb of the first polypeptide binds to a different epitope of the antigen as the sdAb of the second polypeptide. In yet other instances, the sdAb of the first polypeptide binds a different antigen than the sdAb of the second polypeptide. In some embodiments, provided herein is a cell that expresses (produces or secretes) a first polypeptide comprising a structure of sdAb(TTA)-DL-hFR1-vhCDR1-vhFR2-vhCDR2-vhFR3-vhCDR3-vhFR4-CL-vlFR1-vliCDR1-vlFR2-vliCDR2-vlFR3-vliCDR3-vlFR4 and a second polypeptide comprising a structure of sdAb(TTA)-DL-vlFR1-vlCDR1-vlFR2-vlCDR2-vlFR3-vlCDR3-vlFR4-CL-vhFR1-vhiCDR1-vhFR2-vhiCDR2-vhFR3-vhiCDR3-vhFR4.

In some embodiments, the host cell comprises an expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:5, as shown in FIG. 18A and/or a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:7, as shown in FIG. 19A. In some embodiments, the host cell comprises an expression vector comprising a first polynucleotide encoding a first polypeptide comprising a structure shown in FIG. 18A and/or a second polynucleotide encoding a second polypeptide comprising a structure shown in FIG. 19A. In certain embodiments, the expression vector comprises a first nucleic acid sequence shown in FIG. 18B and/or a second nucleic acid sequence shown in FIG. 19B. In some embodiments, the expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:6 and/or a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:8. In some embodiments, the expression vector comprises a nucleic acid sequence encoding Pro16 such as SEQ ID NO: 6 and/or a nucleic acid sequence encoding Pro19 such as SEQ ID NO:8.

In some embodiments, the host cell comprises a first expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:5, as shown in FIG. 18A and a second expression vector comprising a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:7, as shown in FIG. 19A. In some embodiments, the host cell comprises a first expression vector comprising a first polynucleotide encoding a first polypeptide comprising a structure shown in FIG. 18A and a second expression vector comprising a second polynucleotide encoding a second polypeptide comprising a structure shown in FIG. 19A. In certain embodiments, the first expression vector comprises a first nucleic acid sequence shown in FIG. 18B and the first expression vector comprises a second nucleic acid sequence shown in FIG. 19B. In some embodiments, the first expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:6 and the second expression vector comprises a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO: 8. In some embodiments, the first expression vector comprises a nucleic acid sequence encoding Pro16 such as SEQ ID NO: 6 and the second expression vector comprises nucleic acid sequence encoding Pro19 such as SEQ ID NO:8.

In some embodiments, the host cell comprises an expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:9, as shown in FIG. 20A and/or a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:11, as shown in FIG. 21A. In some embodiments, the host cell comprises an expression vector comprising a first polynucleotide encoding a first polypeptide comprising a structure shown in FIG. 20A and/or a second polynucleotide encoding a second polypeptide comprising a structure shown in FIG. 21A. In certain embodiments, the expression vector comprises a first nucleic acid sequence shown in FIG. 20B and/or a second nucleic acid sequence shown in FIG. 21B. In some embodiments, the expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:10 and/or a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:12. In some embodiments, the expression vector comprises a nucleic acid sequence encoding Pro39 such as SEQ ID NO:10 and/or a nucleic acid sequence encoding Pro40 such as SEQ ID NO:12.

In some embodiments, the host cell comprises a first expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:9, as shown in FIG. 20A and a second expression vector comprising a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:11, as shown in FIG. 21A. In some embodiments, the host cell comprises a first expression vector comprising a first polynucleotide encoding a first polypeptide comprising a structure shown in FIG. 20A and a second expression vector comprising a second polynucleotide encoding a second polypeptide comprising a structure shown in FIG. 21A. In certain embodiments, the first expression vector comprises a first nucleic acid sequence shown in FIG. 20B and the first expression vector comprises a second nucleic acid sequence shown in FIG. 21B. In some embodiments, the first expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:10 and the second expression vector comprises a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:12. In some embodiments, the first expression vector comprises a nucleic acid sequence encoding Pro39 such as SEQ ID NO:10 and the second expression vector comprises nucleic acid sequence encoding Pro40 such as SEQ ID NO:12.

In some embodiments, the host cell comprises an expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:13, as shown in FIG. 22A and/or a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:15, as shown in FIG. 23A. In some embodiments, the host cell comprises an expression vector comprising a first polynucleotide encoding a first polypeptide comprising a structure shown in FIG. 22A and/or a second polynucleotide encoding a second polypeptide comprising a structure shown in FIG. 23A. In certain embodiments, the expression vector comprises a first nucleic acid sequence shown in FIG. 22B and/or a second nucleic acid sequence shown in FIG. 23B. In some embodiments, the expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:14 and/or a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:16. In some embodiments, the expression vector comprises a nucleic acid sequence encoding Pro41 such as SEQ ID NO:14 and/or a nucleic acid sequence encoding Pro42 such as SEQ ID NO:16.

In some embodiments, the host cell comprises a first expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:13, as shown in FIG. 22A and a second expression vector comprising a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:15, as shown in FIG. 23A. In some embodiments, the host cell comprises a first expression vector comprising a first polynucleotide encoding a first polypeptide comprising a structure shown in FIG. 22A and a second expression vector comprising a second polynucleotide encoding a second polypeptide comprising a structure shown in FIG. 23A. In certain embodiments, the first expression vector comprises a first nucleic acid sequence shown in FIG. 22B and the first expression vector comprises a second nucleic acid sequence shown in FIG. 23B. In some embodiments, the first expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:14 and the second expression vector comprises a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:16. In some embodiments, the first expression vector comprises a nucleic acid sequence encoding Pro41 such as SEQ ID NO:14 and the second expression vector comprises nucleic acid sequence encoding Pro42 such as SEQ ID NO:16.

In some embodiments, the host cell comprises an expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:17, as shown in FIG. 24A and/or a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:19, as shown in FIG. 25A. In some embodiments, the host cell comprises an expression vector comprising a first polynucleotide encoding a first polypeptide comprising a structure shown in FIG. 24A and/or a second polynucleotide encoding a second polypeptide comprising a structure shown in FIG. 25A. In certain embodiments, the expression vector comprises a first nucleic acid sequence shown in FIG. 24B and/or a second nucleic acid sequence shown in FIG. 25B. In some embodiments, the expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:18 and/or a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:20. In some embodiments, the expression vector comprises a nucleic acid sequence encoding Pro43 such as SEQ ID NO:18 and/or a nucleic acid sequence encoding Pro44 such as SEQ ID NO:20.

In some embodiments, the host cell comprises a first expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:17, as shown in FIG. 24A and a second expression vector comprising a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:19, as shown in FIG. 25A. In some embodiments, the host cell comprises a first expression vector comprising a first polynucleotide encoding a first polypeptide comprising a structure shown in FIG. 24A and a second expression vector comprising a second polynucleotide encoding a second polypeptide comprising a structure shown in FIG. 25A. In certain embodiments, the first expression vector comprises a first nucleic acid sequence shown in FIG. 24B and the first expression vector comprises a second nucleic acid sequence shown in FIG. 25B. In some embodiments, the first expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:18 and the second expression vector comprises a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:20. In some embodiments, the first expression vector comprises a nucleic acid sequence encoding Pro43 such as SEQ ID NO:18 and the second expression vector comprises nucleic acid sequence encoding Pro44 such as SEQ ID NO:20.

In some embodiments, the host cell comprises an expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:21, as shown in FIG. 26A and/or a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:23, as shown in FIG. 27A. In some embodiments, the host cell comprises an expression vector comprising a first polynucleotide encoding a first polypeptide comprising a structure shown in FIG. 26A and/or a second polynucleotide encoding a second polypeptide comprising a structure shown in FIG. 27A. In certain embodiments, the expression vector comprises a first nucleic acid sequence shown in FIG. 26B and/or a second nucleic acid sequence shown in FIG. 27B. In some embodiments, the expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:22 and/or a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:24. In some embodiments, the expression vector comprises a nucleic acid sequence encoding Pro45 such as SEQ ID NO:22 and/or a nucleic acid sequence encoding Pro46 such as SEQ ID NO:24.

In some embodiments, the host cell comprises a first expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:21 as shown in FIG. 26A and a second expression vector comprising a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:23, as shown in FIG. 27A. In some embodiments, the host cell comprises a first expression vector comprising a first polynucleotide encoding a first polypeptide comprising a structure shown in FIG. 26A and a second expression vector comprising a second polynucleotide encoding a second polypeptide comprising a structure shown in FIG. 27A. In certain embodiments, the first expression vector comprises a first nucleic acid sequence shown in FIG. 26B and the first expression vector comprises a second nucleic acid sequence shown in FIG. 27B. In some embodiments, the first expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:22 and the second expression vector comprises a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:24. In some embodiments, the first expression vector comprises a nucleic acid sequence encoding Pro45 such as SEQ ID NO:22 and the second expression vector comprises nucleic acid sequence encoding Pro46 such as SEQ ID NO:24.

In some embodiments, the host cell comprises an expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:25, as shown in FIG. 28A and/or a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:27, as shown in FIG. 29A. In some embodiments, the host cell comprises an expression vector comprising a first polynucleotide encoding a first polypeptide comprising a structure shown in FIG. 28A and/or a second polynucleotide encoding a second polypeptide comprising a structure shown in FIG. 29A. In certain embodiments, the expression vector comprises a first nucleic acid sequence shown in FIG. 28B and/or a second nucleic acid sequence shown in FIG. 29B. In some embodiments, the expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:26 and/or a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:28. In some embodiments, the expression vector comprises a nucleic acid sequence encoding Pro349 such as SEQ ID NO:26 and/or a nucleic acid sequence encoding Pro353 such as SEQ ID NO:28.

In some embodiments, the host cell comprises a first expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity, to SEQ ID NO:25 as shown in FIG. 28A and a second expression vector comprising a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity, to SEQ ID NO:27, as shown in FIG. 29A. In some embodiments, the host cell comprises a first expression vector comprising a first polynucleotide encoding a first polypeptide comprising a structure shown in FIG. 28A and a second expression vector comprising a second polynucleotide encoding a second polypeptide comprising a structure shown in FIG. 29A. In certain embodiments, the first expression vector comprises a first nucleic acid sequence shown in FIG. 28B and the first expression vector comprises a second nucleic acid sequence shown in FIG. 29B. In some embodiments, the first expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:26 and the second expression vector comprises a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% or more sequence identity, to SEQ ID NO:28. In some embodiments, the first expression vector comprises a nucleic acid sequence encoding Pro349 such as SEQ ID NO:26 and the second expression vector comprises nucleic acid sequence encoding Pro353 such as SEQ ID NO:28.

In some aspects, the cell of the invention expresses (produces or secretes) any one of the polypeptide pairs or hemi-COBRAs described herein. In some embodiments, the cell expresses (produces or secretes) Pro16 and Pro19. In certain embodiments, the cell expresses (produces or secretes) Pro39 and Pro40. In particular embodiments, the cell expresses (produces or secretes) Pro41 and Pro42. In some embodiments, the cell expresses (produces or secretes) Pro43 and Pro44. In some embodiments, the cell expresses (produces or secretes) Pro45 and Pro46. In certain embodiments, the cell expresses (produces or secretes) Pro349 and Pro353.

In some embodiments, any one of the polypeptides described herein also comprises a half life extension domain. In certain instances, the half life extension domain such as an anti-HSA domain is located at the C-terminal end of the pseudo heavy chain domain, pseudo light chain domain, or polypeptide. In some embodiments, any one of the polypeptides described herein also comprises a protein tag that is useful for purification. In some embodiments, any one of the polypeptides comprises a histidine (His6) tag, a streptavidin (StrepII) tag, a fragment thereof, or a variant thereof.

C. Stable Cell Expressing Polypeptide Pairs

The complementary pair of proteins that bind to CD3 and one or more tumor target antigens (TTAs) as described herein can be produced by co-expressing polynucleotides encoding each polypeptide of the complementary pair in a host cell and co-purifying the complementary pair. In some embodiments, the cell produces and secretes the complementary pair of proteins. In some embodiments, the first polypeptide and the second polypeptide of the complementary pair of proteins are produced at an about equimolar ratio (e.g., an about 1:1 ratio). In other words, the host cell produces a substantially equivalent (a near equal or equal) amount of the first and second polypeptides. In other embodiments, the first polypeptide and the second polypeptide are produced at a ratio that is not equimolar (e.g., not an about 1:1 ratio).

In some embodiments, the first polynucleotide and the second polynucleotide are introduced into a host cell at a polynucleotide ratio of about 1:1. In other embodiments, the amount of the first polynucleotide (or first expression vector) encoding the first polypeptide and the amount of the second polynucleotide (or expression vector) encoding the second polypeptide are introduced into a host cell in a ratio that is not 1:1, such as a ratio that is greater than 1:1 or a ratio that is less than 1:1. In some embodiments, the first polynucleotide and the second polynucleotide is introduced into a host cell at a polynucleotide ratio selected from the group consisting of a ratio of 8:2, 7:3, 6:4, 5:5, 4:6, 3:7, 2:8, 50:1, 45:1, 40:1, 35:1, 30:1, 25:1, 20:1, 15:1 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, and 1:50. In some embodiments, the polynucleotide ratio is selected from the group consisting of 8:2, 7:3, 4:6, 5:5, 4:6, and 3:7 of the first polynucleotide encoding a first polypeptide described herein and the second polynucleotide encoding a second polypeptide described herein, respectively. In some embodiments, the polynucleotide ratio is selected from the group consisting of 2:8, 3:7, 4:6, 5:5, 6:4, and 7:3 of the first polynucleotide encoding a first polypeptide described herein and the second polynucleotide encoding a second polypeptide described herein, respectively.

In some embodiments, the polynucleotide ratio introduced into a host cell described herein is 2:8, 3:7, 4:6, 5:5, 6:4, and 7:3 of the first polynucleotide encoding a first polypeptide and the second polynucleotide encoding a second polypeptide, respectively, wherein the first polypeptide comprises an amino acid sequence having at least 90% sequence identity to SEQ ID NO:2, SEQ ID NO:5, SEQ ID NO:9, SEQ ID NO:13, SEQ ID NO:17, SEQ ID NO:21, or SEQ ID NO:25, and the second polypeptide comprises an amino acid sequence having at least 90% sequence identity to SEQ ID NO:3, SEQ ID NO:7, SEQ ID NO:11, SEQ ID NO:15, SEQ ID NO:19, SEQ ID NO:23, or SEQ ID NO:27. In some embodiments, the polynucleotide ratio is 2:8, 3:7, 4:6, 5:5, 6:4, and 7:3 of the first polynucleotide encoding a first polypeptide and the second polynucleotide encoding a second polypeptide, respectively, wherein the first polypeptide is selected from the group consisting of Pro6 having SEQ ID NO:2, Pro16 having SEQ ID NO:5, Pro39 having SEQ ID NO:9, Pro41 having SEQ ID NO:13, Pro43 having SEQ ID NO:17, Pro45 having SEQ ID NO:21, and Pro349 having SEQ ID NO:25, and the second polypeptide is selected from the group consisting of Pro7 having SEQ ID NO:3, Pro19 having SEQ ID NO:7, Pro40 having SEQ ID NO:11, Pro42 having SEQ ID NO:15, Pro44 having SEQ ID NO:19, Pro46 having SEQ ID NO:23, and Pro353 having SEQ ID NO:27.

In some embodiments, the polynucleotide ratio introduced into a host cell is 2:8, 3:7, 4:6, 5:5, 6:4, and 7:3 of the first polynucleotide encoding a first polypeptide and the second polynucleotide encoding a second polypeptide, respectively, wherein the first polypeptide and second polypeptide pairs (e.g., first polypeptide/second polypeptide) are selected from Pro6/Pro7, Pro16/Pro19, Pro39/Pro40, Pro41/Pro42, Pro43/Pro44, Pro45/Pro46, and Pro349/Pro353.

In some embodiments, the polynucleotide ratio introduced into a host cell is 2:8, 3:7, 4:6, 5:5, 6:4, and 7:3 of the first polynucleotide encoding a first polypeptide and the second polynucleotide encoding a second polypeptide, respectively, wherein the first polypeptide comprises an amino acid sequence having at least 90% sequence identity e.g., 90%, 91%, 92%, 93%, 94%, 95%, 96%, 97%, 98%, 99% or more sequence identity, to SEQ ID NO:5 and the second polypeptide comprises an amino acid sequence having at least 90% sequence identity e.g., 90%, 91%, 92%, 93%, 94%, 95%, 96%, 97%, 98%, 99% or more sequence identity, to SEQ ID NO:7.

In some embodiments, the polynucleotide ratio introduced into a host cell is 2:8, 3:7, 4:6, 5:5, 6:4, and 7:3 of the first polynucleotide encoding a first polypeptide and the second polynucleotide encoding a second polypeptide, respectively, wherein the first polypeptide comprises an amino acid sequence having at least 90% sequence identity, e.g., 90%, 91%, 92%, 93%, 94%, 95%, 96%, 97%, 98%, 99% or more sequence identity, to SEQ ID NO:9 and the second polypeptide comprises an amino acid sequence having at least 90% sequence identity, e.g., 90%, 91%, 92%, 93%, 94%, 95%, 96%, 97%, 98%, 99% or more sequence identity, to SEQ ID NO:11. In some embodiments, the polynucleotide ratio is 2:8, 3:7, 4:6, 5:5, 6:4, and 7:3 of the first polynucleotide encoding a first polypeptide and the second polynucleotide encoding a second polypeptide, respectively, wherein the first polynucleotide comprises a nucleic acid sequence having at least 85% sequence identity to SEQ ID NO:10 and the second polynucleotide comprises a nucleic acid sequence having at least 85% sequence identity to SEQ ID NO:12.

In some embodiments, the polynucleotide ratio introduced into a host cell is 2:8, 3:7, 4:6, 5:5, 6:4, and 7:3 of the first polynucleotide encoding a first polypeptide and the second polynucleotide encoding a second polypeptide, respectively, wherein the first polypeptide comprises an amino acid sequence having at least 90% sequence identity, e.g., 90%, 91%, 92%, 93%, 94%, 95%, 96%, 97%, 98%, 99% or more sequence identity, to SEQ ID NO:13 and the second polypeptide comprises an amino acid sequence having at least 90% sequence identity, e.g., 90%, 91%, 92%, 93%, 94%, 95%, 96%, 97%, 98%, 99% or more sequence identity, to SEQ ID NO:15. In some embodiments, the polynucleotide ratio is 2:8, 3:7, 4:6, 5:5, 6:4, and 7:3 of the first polynucleotide encoding a first polypeptide and the second polynucleotide encoding a second polypeptide, respectively, wherein the first polynucleotide comprises a nucleic acid sequence having at least 85% sequence identity to SEQ ID NO:14 and the second polynucleotide comprises a nucleic acid sequence having at least 85% sequence identity to SEQ ID NO:16.

In some embodiments, the polynucleotide ratio introduced into a host cell is 2:8, 3:7, 4:6, 5:5, 6:4, and 7:3 of the first polynucleotide encoding a first polypeptide and the second polynucleotide encoding a second polypeptide, respectively, wherein the first polypeptide comprises an amino acid sequence having at least 90% sequence identity, e.g., 90%, 91%, 92%, 93%, 94%, 95%, 96%, 97%, 98%, 99% or more sequence identity, to SEQ ID NO:17 and the second polypeptide comprises an amino acid sequence having at least 90% sequence identity, e.g., 90%, 91%, 92%, 93%, 94%, 95%, 96%, 97%, 98%, 99% or more sequence identity, to SEQ ID NO:19. In some embodiments, the polynucleotide ratio is 2:8, 3:7, 4:6, 5:5, 6:4, and 7:3 of the first polynucleotide encoding a first polypeptide and the second polynucleotide encoding a second polypeptide, respectively, wherein the first polynucleotide comprises a nucleic acid sequence having at least 85% sequence identity to SEQ ID NO:18 and the second polynucleotide comprises a nucleic acid sequence having at least 85% sequence identity to SEQ ID NO:20.

In some embodiments, the polynucleotide ratio introduced into a host cell is 2:8, 3:7, 4:6, 5:5, 6:4, and 7:3 of the first polynucleotide encoding a first polypeptide and the second polynucleotide encoding a second polypeptide, respectively, wherein the first polypeptide comprises an amino acid sequence having at least 90% sequence identity, e.g., 90%, 91%, 92%, 93%, 94%, 95%, 96%, 97%, 98%, 99% or more sequence identity, to SEQ ID NO:21 and the second polypeptide comprises an amino acid sequence having at least 90% sequence identity, e.g., 90%, 91%, 92%, 93%, 94%, 95%, 96%, 97%, 98%, 99% or more sequence identity, to SEQ ID NO:23. In some embodiments, the polynucleotide ratio is 2:8, 3:7, 4:6, 5:5, 6:4, and 7:3 of the first polynucleotide encoding a first polypeptide and the second polynucleotide encoding a second polypeptide, respectively, wherein the first polynucleotide comprises a nucleic acid sequence having at least 85% sequence identity to SEQ ID NO:22 and the second polynucleotide comprises a nucleic acid sequence having at least 85% sequence identity to SEQ ID NO:24.

In some embodiments, the polynucleotide ratio introduced into a host cell is 2:8, 3:7, 4:6, 5:5, 6:4, and 7:3 of the first polynucleotide encoding a first polypeptide and the second polynucleotide encoding a second polypeptide, respectively, wherein the first polypeptide comprises an amino acid sequence having at least 90% sequence identity, e.g., 90%, 91%, 92%, 93%, 94%, 95%, 96%, 97%, 98%, 99% or more sequence identity, to SEQ ID NO:25 and the second polypeptide comprises an amino acid sequence having at least 90% sequence identity, e.g., 90%, 91%, 92%, 93%, 94%, 95%, 96%, 97%, 98%, 99% or more sequence identity, to SEQ ID NO:27. In some embodiments, the polynucleotide ratio is 2:8, 3:7, 4:6, 5:5, 6:4, and 7:3 of the first polynucleotide encoding a first polypeptide and the second polynucleotide encoding a second polypeptide, respectively, wherein the first polynucleotide comprises a nucleic acid sequence having at least 85% sequence identity to SEQ ID NO:26 and the second polynucleotide comprises a nucleic acid sequence having at least 85% sequence identity to SEQ ID NO:28.

### IV. Proteins of the Invention

The proteins of the invention have a number of different components, generally referred to herein as domains, that are linked together in a variety of ways. Some of the domains are binding domains, that each bind to a target antigen (e.g., a TTA or CD3, for example). As they bind to more than one antigen, they are referred to herein as “multispecific”; for example, a prodrug construct of the invention may bind to a TTA and CD3, and thus are “bispecific”.

The proteins of the invention can include CD3 antigen binding domains arranged in a variety of ways as outlined herein, tumor target antigen binding domains, half-life extension domains, linkers, etc.

In some embodiments, a first protein comprises a first tumor target antigen domain and a second protein comprises a second tumor target antigen domain such that the first tumor target antigen domain and second tumor target antigen domain bind to the same tumor target antigen. In certain instances, the first tumor target antigen domain and second tumor target antigen domain bind different epitopes, regions, or portions of the same tumor target antigen. In some instances, the first tumor target antigen domain and second tumor target antigen domain bind different tumor target antigens.

The proteins of the invention can be produced by co-expression in a cell and co-purification to obtain a complementary pair of proteins that can bind to CD3 and a tumor target antigen. In some embodiments, each of the complementary pair of proteins are purified separately. In some embodiments, each of the complementary pair of proteins are purified simultaneously or concomitantly.

In some embodiments, an expression vector comprises a nucleic acid sequence encoding one protein of the complementary pair of proteins and a nucleic acid sequence encoding the other protein of the complementary pair of proteins. In some embodiments, a host cell comprises such an expression vector. In some instances, such a host cell can be cultured under suitable conditions in a culture media to produce the proteins. In some embodiments, the host cell is cultured under suitable conditions to secrete the proteins described herein into the culture media. In certain embodiments, the culture media comprising the secreted proteins of the invention is purified to obtain proteins of the complementary pair of proteins. Useful methods of purification include, but are not limited to, protein A chromatography, protein G chromatography, heparin binding, reverse phase chromatography, HIC chromatography, CHT chromatography affinity chromatography, anion exchange chromatography, cation exchange chromatography, size exclusion chromatography, and the like.

D. CD3 Antigen Binding Domains

The specificity of the response of T cells is mediated by the recognition of antigen (displayed in context of a major histocompatibility complex, MHC) by the T cell receptor complex. As part of the T cell receptor complex, CD3 is a protein complex that includes a CD3γ (gamma) chain, a CD3δ (delta) chain, and two CD3ε (epsilon) chains which are present on the cell surface. CD3 associates with the α (alpha) and β (beta) chains of the T cell receptor (TCR) as well as and CD-ζ (zeta) altogether to comprise the T cell receptor complex. Clustering of CD3 on T cells, such as by Fv domains that bind to CD3 leads to T cell activation similar to the engagement of the T cell receptor but independent of its clone-typical specificity.

However, as is known in the art, CD3 activation can cause a number of toxic side effects, and accordingly the present invention is directed to providing active CD3 binding of the polypeptides of the invention only in the presence of tumor cells, where specific proteases are found, that then cleave the prodrug polypeptides of the invention to provide an active CD3 binding domain. Thus, in the present invention, binding of an anti-CD3 Fv domain to CD3 is regulated by a protease cleavage domain which restricts binding of the CD3 Fv domain to CD3 only in the microenvironment of a diseased cell or tissue with elevated levels of proteases, for example in a tumor microenvironment as is described herein.

Accordingly, the present invention provides two sets of VH and VL domains, an active set (VH and VL) and an inactive set (VHi and VLi) with all four being present in the prodrug construct(s). The construct is formatted such that the VH and VL set cannot self-associate, but rather associates with an inactive partner, e.g. VHi and VL and VLi and VH as is shown herein.

There are a number of suitable active CDR sets, and/or VH and VL domains, that are known in the art that find use in the present invention. For example, the CDRs and/or VH and VL domains are derived from known anti-CD3 antibodies, such as, for example, muromonab-CD3 (OKT3), otelixizumab (TRX4), teplizumab (MGA031), visilizumab (Nuvion), SP34 or I2C, TR-66 or X35-3, VIT3, BMA030 (BW264/56), CLB-T3/3, CRIS7, YTH12.5, F111-409, CLB-T3.4.2, TR-66, WT32, SPv-T3b, 11D8, XIII-141, XIII-46, XIII-87, 12F6, T3/RW2-8C8, T3/RW2-4B6, OKT3D, M-T301, SMC2, F101.01, UCHT-1 and WT-31.

In some embodiments, the VH sequences that form an active Fv domain that binds to human CD3 when in close proximity to an active VL domain are shown in FIG. 16A as Pro6, FIG. 18A as Pro16, FIG. 20A as Pro39, FIG. 22A as Pro41, FIG. 24A as Pro43, FIG. 26A as Pro45, or FIG. 28A as Pro349. The amino acid sequence of an active VH domain is SEQ ID NO:102, as shown in FIG. 39.

In some embodiments, the VL sequences that form an active Fv domain that binds to human CD3 when in close proximity to an active VH domain are shown in FIG. 17A as Pro7, FIG. 19A as Pro19, FIG. 21A as Pro40, FIG. 23A as Pro42, FIG. 25A as Pro44, FIG. 27A as Pro46, or FIG. 29A as Pro353. The amino acid sequence of an active VL domain is SEQ ID NO:90, as shown in FIG. 38.

The inactive VHi and VLi domains contain “regular” framework regions (FRs) that allow association, such that an inactive variable domain will associate with an active variable domain, rendering the pair inactive, e.g., unable to bind CD3. In one embodiment, the VHi and VLi that form inactive Fv domains when one or both of the inactive domains are present in a complementary construct pair. In some embodiments, amino acid sequences of an inactive VLi domain are shown in FIG. 16A as Pro6, FIG. 18A as Pro16, FIG. 20A as Pro39, FIG. 22A as Pro41, FIG. 24A as Pro43, FIG. 26A as Pro45, and FIG. 28A as Pro349. In some instances, the amino acid sequence of an inactive VL domain is SEQ ID NO:94, as shown in FIG. 38. In other instances, the amino acid sequence of an inactive VL domain is SEQ ID NO:98, as shown in FIG. 38. In some embodiments, amino acid sequences of an inactive VHi domain are shown in FIG. 17A as Pro7, FIG. 19A as Pro19, FIG. 21A as Pro40, FIG. 23A as Pro42, FIG. 25A as Pro44, FIG. 27A as Pro46, and FIG. 29A as Pro353. In some instances, the amino acid sequence of an inactive VH domain is SEQ ID NO:106, as shown in FIG. 39. In other instances, the amino acid sequence of an inactive VL domain is SEQ ID NO:110, as shown in FIG. 39.

In some embodiments, the inactive VHi domain comprises one or more, e.g., 1, 2, 3, 4, 5, 6, 7, 8, 9, or more, amino acid modifications (e.g., amino acid insertions, deletions, or substitutions) that when paired with an active VL domain renders the paired VHi-VL domain unable to bind the target antigen. In other embodiments, the inactive VLi domain comprises one or more, e.g., 1, 2, 3, 4, 5, 6, 7, 8, 9, or more, amino acid modifications (e.g., amino acid insertions, deletions, or substitutions) that when paired with an active VH domain renders the paired VH-VLi unable to bind the target antigen.

As will be appreciated by those in the art, there are a number of “inactive” variable domains that find use in the invention. Basically, any variable domain with human framework regions that allows self-assembly with another variable domain, no matter what amino acids are in the CDR location in the variable region can be used. For clarity, the inactive domains are said to include CDRs, although technically the inactive variable domains do not confer binding capabilities.

In some cases, the inactive domains can be engineered to promote selective binding in the prodrug format, to encourage formation of intramolecular VHi-VL and VH-VLi domains prior to cleavage (over, for example, intermolecular pair formation). See, for example, Igawa et al., Protein Eng. Des. Selection 23(8):667-677 (2010), hereby expressly incorporated by reference in its entirety and specifically for the interface residue amino acid substitutions.

In one aspect, the polypeptide constructs described herein comprise a domain which specifically binds to CD3 when activated by a protease. In one aspect, the polypeptide constructs described herein comprise two or more domains which when activated by a protease specifically bind to human CD3. In some embodiments, the polypeptide constructs described herein comprise two or more domains which when activated by a protease which specifically binds to CD3E. In some embodiments, the polypeptide constructs described herein comprise two or more domains which when activated by a protease specifically bind to CD3ε.

In some embodiments, the protease cleavage site is between the anti-CD3 active VH and inactive VL domains on a first monomer and keeps them from folding and binding to CD3 on a T cell. In some embodiments, the protease cleavage site is between the anti-CD3 inactive VH and active VL domains on a second monomer and keeps them from folding and binding to CD3 on a T cell. Once protease cleavage sites are cleaved by a protease present at the target cell, the anti-CD3 active VH domain of the first monomer and the anti-CD3 active VL domain of the second monomer are able to bind to CD3 on a T cell.

In certain embodiments, the CD3 binding domain of the polypeptide constructs described herein exhibit not only potent CD3 binding affinities with human CD3, but show also excellent cross reactivity with the respective cynomolgus monkey CD3 proteins. In some instances, the CD3 binding domain of the polypeptide constructs is cross-reactive with CD3 from cynomolgus monkey. In certain instances, human:cynomolgous KD ratios for CD3 are between 5 and 0.2.

In some embodiments, the CD3 binding domain of the antigen binding protein can be any domain that binds to CD3 including but not limited to domains from a monoclonal antibody, a polyclonal antibody, a recombinant antibody, a human antibody, a humanized antibody. In some instances, it is beneficial for the CD3 binding domain to be derived from the same species in which the antigen binding protein will ultimately be used in. For example, for use in humans, it may be beneficial for the CD3 binding domain of the antigen binding protein to comprise human or humanized residues from the antigen binding domain of an antibody or antibody fragment.

Thus, in one aspect, the antigen-binding domain comprises a humanized or human binding domain. In one embodiment, the humanized or human anti-CD3 binding domain comprises one or more (e.g., all three) light chain complementary determining region 1 (LC CDR1), light chain complementary determining region 2 (LC CDR2), and light chain complementary determining region 3 (LC CDR3) of a humanized or human anti-CD3 binding domain described herein, and/or one or more (e.g., all three) heavy chain complementary determining region 1 (HC CDR1), heavy chain complementary determining region 2 (HC CDR2), and heavy chain complementary determining region 3 (HC CDR3) of a humanized or human anti-CD3 binding domain described herein, e.g., a humanized or human anti-CD3 binding domain comprising one or more, e.g., all three, LC CDRs and one or more, e.g., all three, HC CDRs.

In some embodiments, the humanized or human anti-CD3 binding domain comprises a humanized or human light chain variable region specific to CD3 where the light chain variable region specific to CD3 comprises human or non-human light chain CDRs in a human light chain framework region. In certain instances, the light chain framework region is a λ (lambda) light chain framework. In other instances, the light chain framework region is a κ (kappa) light chain framework.

In some embodiments, one or more CD3 binding domains are humanized or fully human. In some embodiments, one or more activated CD3 binding domains have a KD binding of 1000 nM or less to CD3 on CD3 expressing cells. In some embodiments, one or more activated CD3 binding domains have a KD binding of 100 nM or less to CD3 on CD3 expressing cells. In some embodiments, one or more activated CD3 binding domains have a KD binding of 10 nM or less to CD3 on CD3 expressing cells. In some embodiments, one or more CD3 binding domains have cross-reactivity with cynomolgus CD3. In some embodiments, one or more CD3 binding domains comprise an amino acid sequence provided herein.

In some embodiments, the humanized or human anti-CD3 binding domain comprises a humanized or human heavy chain variable region specific to CD3 where the heavy chain variable region specific to CD3 comprises human or non-human heavy chain CDRs in a human heavy chain framework region.

In one embodiment, the anti-CD3 binding domain is an Fv comprising a light chain and a heavy chain of an amino acid sequence provided herein. In an embodiment, the anti-CD3 binding domain comprises: a light chain variable region comprising an amino acid sequence having at least one, two or three modifications (e.g., substitutions, insertions, and deletions) but not more than 30, 20 or 10 modifications (e.g., substitutions, insertions, and deletions) of an amino acid sequence of a light chain variable region provided herein, or a sequence with 95-99% identity with an amino acid sequence provided herein; and/or a heavy chain variable region comprising an amino acid sequence having at least one, two or three modifications (e.g., substitutions, insertions, and deletions) but not more than 30, 20 or 10 modifications (e.g., substitution, insertions, and deletions s) of an amino acid sequence of a heavy chain variable region provided herein, or a sequence with 95-99% identity to an amino acid sequence provided herein. In one embodiment, the humanized or human anti-CD3 binding domain is a scFv, and a light chain variable region comprising an amino acid sequence described herein, is attached to a heavy chain variable region comprising an amino acid sequence described herein, via a scFv linker. The light chain variable region and heavy chain variable region of a scFv can be, e.g., in any of the following orientations: light chain variable region-scFv linker-heavy chain variable region or heavy chain variable region-scFv linker-light chain variable region.

In some embodiments, CD3 binding domain of an antigen binding protein has an affinity to CD3 on CD3-expressing cells with a KD of 1000 nM or less, 100 nM or less, 50 nM or less, 20 nM or less, 10 nM or less, 5 nM or less, 1 nM or less, or 0.5 nM or less. In some embodiments, the CD3 binding domain of an antigen binding protein has an affinity to CD3E with a KD of 1000 nM or less, 100 nM or less, 50 nM or less, 20 nM or less, 10 nM or less, 5 nM or less, 1 nM or less, or 0.5 nM or less. In further embodiments, CD3 binding domain of an antigen binding protein has low affinity to CD3, i.e., about 100 nM or greater.

The affinity to bind to CD3 can be determined, for example, by the ability of the antigen binding protein itself or its CD3 binding domain to bind to CD3 coated on an assay plate; displayed on a microbial cell surface; in solution; etc., as is known in the art, generally using Biacore or Octet assays. The binding activity of the antigen binding protein itself or its CD3 binding domain of the present disclosure to CD3 can be assayed by immobilizing the ligand (e.g., CD3) or the antigen binding protein itself or its CD3 binding domain, to a bead, substrate, cell, etc. Agents can be added in an appropriate buffer and the binding partners incubated for a period of time at a given temperature. After washes to remove unbound material, the bound protein can be released with, for example, SDS, buffers with a high pH, and the like and analyzed, for example, by Surface Plasmon Resonance (SPR).

E. Antigen Binding Domains to Tumor Target Antigens

In addition to the described CD3 and half-life extension domains, the polypeptide constructs described herein also comprise at least one or at least two, or more domains that bind to one or more target antigens or one or more regions on a single target antigen. It is contemplated herein that a polypeptide construct of the invention is cleaved, for example, in a disease-specific microenvironment or in the blood of a subject at the protease cleavage domain and that each target antigen binding domain will bind to a target antigen on a target cell, thereby activating the CD3 binding domain to bind a T cell. In general, the TTA binding domains can bind to their targets before protease cleavage, so they can “wait” on the target cell to be activated as T-cell engagers. At least one target antigen is involved in and/or associated with a disease, disorder or condition. Exemplary target antigens include those associated with a proliferative disease, a tumorous disease, an inflammatory disease, an immunological disorder, an autoimmune disease, an infectious disease, a viral disease, an allergic reaction, a parasitic reaction, a graft-versus-host disease or a host-versus-graft disease. In some embodiments, a target antigen is a tumor antigen expressed on a tumor cell. Alternatively in some embodiments, a target antigen is associated with a pathogen such as a virus or bacterium. At least one target antigen may also be directed against healthy tissue.

In some embodiments, a target antigen is a cell surface molecule such as a protein, lipid or polysaccharide. In some embodiments, a target antigen is a on a tumor cell, virally infected cell, bacterially infected cell, damaged red blood cell, arterial plaque cell, or fibrotic tissue cell. It is contemplated herein that upon binding more than one target antigen, two inactive CD3 binding domains are co-localized and form an active CD3 binding domain on the surface of the target cell. In some embodiments, the antigen binding protein comprises more than one target antigen binding domain to activate an inactive CD3 binding domain in the antigen binding protein. In some embodiments the antigen binding protein comprises more than one target antigen binding domain to enhance the strength of binding to the target cell. In some embodiments the antigen binding protein comprises more than one target antigen binding domain to enhance the strength of binding to the target cell. In some embodiments, more than one antigen binding domain comprise the same antigen binding domain. In some embodiments, more than one antigen binding domain comprise different antigen binding domains. For example, two different antigen binding domains known to be dually expressed in a diseased cell or tissue, for example a tumor or cancer cell, can enhance binding or selectivity of an antigen binding protein for a target.

Polypeptide constructs contemplated herein include at least one antigen binding domain, wherein the antigen binding domain binds to at least one target antigen. Target antigens, in some cases, are expressed on the surface of a diseased cell or tissue, for example a tumor or a cancer cell. Target antigens include but are not limited to EpCAM, EGFR, FOLR1, B7H3, HER-2, HER-3, c-Met, LyPD3, and CEA. Polypeptide constructs disclosed herein, also include proteins comprising two antigen binding domains that bind to two different target antigens known to be expressed on a diseased cell or tissue. Exemplary pairs of antigen binding domains include but are not limited to EGFR/CEA, EpCAM/CEA, EGFR/EpCAM, EGFR/B7H3, EGFR/FOLR1, EpCAM/B7H3, EpCAM/FOLR1, B7H3/FOLR1, and HER-2/HER-3.

The design of the polypeptide constructs described herein allows the binding domain to one or more target antigens to be flexible in that the binding domain to a target antigen can be any type of binding domain, including but not limited to, domains from a monoclonal antibody, a polyclonal antibody, a recombinant antibody, a human antibody, a humanized antibody. In some embodiments, the binding domain to a target antigen is a single chain variable fragment (scFv), single-domain antibody such as a heavy chain variable domain (VH), a light chain variable domain (VL) and a variable domain (VHH) of camelid derived nanobody. In other embodiments, the binding domain to a target antigen is a non-Ig binding domain, i.e., antibody mimetic, such as anticalins, affilins, affibody molecules, affimers, affitins, alphabodies, avimers, DARPins, fynomers, kunitz domain peptides, and monobodies. In further embodiments, the binding domain to one or more target antigens is a ligand, a receptor domain, a lectin, or peptide that binds to or associates with one or more target antigens.

In some embodiments, the target cell antigen binding domains independently comprise a scFv, a VH domain, a VL domain, a non-Ig domain, or a ligand that specifically binds to the target antigen. In some embodiments, the target antigen binding domains specifically bind to a cell surface molecule. In some embodiments, the target antigen binding domains specifically bind to a tumor antigen. In some embodiments, the target antigen binding domains specifically and independently bind to an antigen selected from at least one of EpCAM, EGFR, B7H3, HER-2, HER-3, cMet, LyPD3, CEA, and FOLR1. In some embodiments, the target antigen binding domains specifically and independently bind to two different antigens, wherein at least one of the antigens is selected from one of EpCAM, EGFR, B7H3, HER-2, HER-3, cMet, CEA, LyPD3, and FOLR1. In some embodiments, the protein prior to cleavage of the protease cleavage domain is less than about 100 kDa. In some embodiments, the protein after cleavage of the protease cleavage domain is about 25 to about 75 kDa. In some embodiments, the protein prior to protease cleavage has a size that is above the renal threshold for first-pass clearance. In some embodiments, the protein prior to protease cleavage has an elimination half-time of at least about 50 hours. In some embodiments, the protein prior to protease cleavage has an elimination half-time of at least about 100 hours. In some embodiments, the protein has increased tissue penetration as compared to an IgG to the same target antigen. In some embodiments, the protein has increased tissue distribution as compared to an IgG to the same target antigen.

In some embodiments, the human TTA of the invention selected from the group consisting of human EGFR, human B7H3, human EpCAM, and human FOLR1. Any one of the polypeptides described herein can include an sdAb that binds a human TTA selected from the group consisting of human EGFR, human B7H3, human EpCAM, and human FOLR1.

F. Half Life Extension

The proteins of the invention optionally include half-life extension domains. Such domains are contemplated to include but are not limited to HSA binding domains, Fc domains, small molecules, and other half-life extension domains known in the art.

Human serum albumin (HSA) (molecular mass ˜67 kDa) is the most abundant protein in plasma, present at about 50 mg/ml (600 μM), and has a half-life of around 20 days in humans. HSA serves to maintain plasma pH, contributes to colloidal blood pressure, functions as carrier of many metabolites and fatty acids, and serves as a major drug transport protein in plasma.

Noncovalent association with albumin extends the elimination half-time of short lived proteins. For example, a recombinant fusion of an albumin binding domain to a Fab fragment resulted in a reduced in vivo clearance of 25- and 58-fold and a half-life extension of 26- and 37-fold when administered intravenously to mice and rabbits respectively as compared to the administration of the Fab fragment alone. In another example, when insulin is acylated with fatty acids to promote association with albumin, a protracted effect was observed when injected subcutaneously in rabbits or pigs. Together, these studies demonstrate a linkage between albumin binding and prolonged action.

In one aspect, the antigen-binding proteins described herein comprise a half-life extension domain, for example a domain which specifically binds to HSA. In some embodiments, the HSA binding domain of an antigen binding protein can be any domain that binds to HSA including but not limited to domains from a monoclonal antibody, a polyclonal antibody, a recombinant antibody, a human antibody, a humanized antibody. In some embodiments, the HSA binding domain is a single chain variable fragments (scFv), single-domain antibody such as a heavy chain variable domain (VH), a light chain variable domain (VL) and a variable domain (VHH) of camelid derived nanobody, peptide, ligand or small molecule specific for HSA. In certain embodiments, the HSA binding domain is a single-domain antibody (sdAb). In other embodiments, the HSA binding domain is a peptide. In further embodiments, the HSA binding domain is a small molecule. It is contemplated that the HSA binding domain of an antigen binding protein is fairly small and no more than 25 kD, no more than 20 kD, no more than 15 kD, or no more than 10 kD in some embodiments. In certain instances, the HSA binding domain is 5 kD or less if it is a peptide or small molecule.

The half-life extension domain of an antigen binding protein provides for altered pharmacodynamics and pharmacokinetics of the antigen binding protein itself. As above, the half-life extension domain extends the elimination half-time. The half-life extension domain also alters pharmacodynamic properties including alteration of tissue distribution, penetration, and diffusion of the antigen-binding protein. In some embodiments, the half-life extension domain provides for improved tissue (including tumor) targeting, tissue penetration, tissue distribution, diffusion within the tissue, and enhanced efficacy as compared with a protein without a half-life extension binding domain. In one embodiment, therapeutic methods effectively and efficiently utilize a reduced amount of the antigen-binding protein, resulting in reduced side effects, such as reduced non-tumor cell cytotoxicity.

Further, characteristics of the half-life extension domain, for example a HSA binding domain, include the binding affinity of the HSA binding domain for HSA. Affinity of the HSA binding domain can be selected so as to target a specific elimination half-time in a particular polypeptide construct. Thus, in some embodiments, the HSA binding domain has a high binding affinity. In other embodiments, the HSA binding domain has a medium binding affinity. In yet other embodiments, the HSA binding domain has a low or marginal binding affinity. Exemplary binding affinities include KD concentrations at 10 nM or less (high), between 10 nM and 100 nM (medium), and greater than 100 nM (low). As above, binding affinities to HSA are determined by known methods such as Surface Plasmon Resonance (SPR).

G. Protease Cleavage Sites

The polypeptide (e.g., protein) compositions of the invention, and particularly the prodrug constructs, include one or more protease cleavage sites, generally resident in cleavable linkers, as outlined herein.

As described herein, the prodrug constructs of the invention include at least one protease cleavage site comprising an amino acid sequence that is cleaved by at least one protease. In some cases, the proteins described herein comprise 1, 2, 3, 4, 5, 6, 7, 8, 9, 10, 11, 12, 13, 14, 15, 16, 17, 18, 19, 20, or more protease cleavage sites that are cleaved by at least one protease. As is more fully discussed herein, when more than one protease cleavage site is used in a prodrug construction, they can be the same (e.g., multiple sites that are cleaved by a single protease) or different (two or more cleavage sites are cleaved by at least two different proteases). As will be appreciated by those in the art, constructs containing three or more protease cleavage sites can utilize one, two, three, etc.; e.g. some constructs can utilize three sites for two different proteases, etc.

The amino acid sequence of the protease cleavage site will depend on the protease that is targeted. As is known in the art, there are a number of human proteases that are found in the body and can be associated with disease states.

Proteases are known to be secreted by some diseased cells and tissues, for example tumor or cancer cells, creating a microenvironment that is rich in proteases or a protease-rich microenvironment. In some cases, the blood of a subject is rich in proteases. In some cases, cells surrounding the tumor secrete proteases into the tumor microenvironment. Cells surrounding the tumor secreting proteases include but are not limited to the tumor stromal cells, myofibroblasts, blood cells, mast cells, B cells, NK cells, regulatory T cells, macrophages, cytotoxic T lymphocytes, dendritic cells, mesenchymal stem cells, polymorphonuclear cells, and other cells. In some cases, proteases are present in the blood of a subject, for example proteases that target amino acid sequences found in microbial peptides. This feature allows for targeted therapeutics such as antigen-binding proteins to have additional specificity because T cells will not be bound by the antigen binding protein except in the protease rich microenvironment of the targeted cells or tissue.

Proteases are proteins that cleave proteins, in some cases, in a sequence-specific manner. Proteases include but are not limited to serine proteases, cysteine proteases, aspartate proteases, threonine proteases, glutamic acid proteases, metalloproteases, asparagine peptide lyases, serum proteases, Cathepsins (e.g., Cathepsin B, Cathepsin C, Cathepsin D, Cathepsin E, Cathepsin K, Cathepsin L, CathepsinS), kallikreins, hK1, hK10, hK15, KLK7, GranzymeB, plasmin, collagenase, Type IV collagenase, stromelysin, factor XA, chymotrypsin-like protease, trypsin-like protease, elastase-like protease, subtilisin-like protease, actinidain, bromelain, calpain, Caspases (e.g., Caspase-3), Mir1-CP, papain, HIV-1 protease, HSV protease, CMV protease, chymosin, renin, pepsin, matriptase, legumain, plasmepsin, nepenthesin, metalloexopeptidases, metalloendopeptidases, matrix metalloproteases (MMP), MMP1, MMP2, MMP3, MMP8, MMP9, MMP13, MMP11, MMP14, meprin, urokinase plasminogen activator (uPA), enterokinase, prostate-specific antigen (PSA, hK3), interleukin-1β converting enzyme, thrombin, FAP (FAP-α), dipeptidyl peptidase, and dipeptidyl peptidase IV (DPPIV/CD26).

Some suitable proteases and protease cleavage sequences are shown in FIG. 14A, FIG. 14B and FIG. 14C.

### V. Expression Methods

Provided herein are methods for producing proteins of the invention by co-expression in a cell (e.g., a host cell) and co-purification to obtain a complementary pair of proteins that can bind to CD3 and one or more tumor target antigens (TTAs). In some embodiments, the complementary pair of proteins (e.g., a first polypeptide and a second polypeptide) are produced at an about equimolar ratio (e.g., an about 1:1 ratio). In other embodiments, the complementary pair of proteins (e.g., a first polypeptide and a second polypeptide) are produced at a ratio that is not equimolar (e.g., not an about 1:1 ratio). In other words, the method described herein can be used to obtain a ratio of the first polypeptide to the second polypeptide such as, but not limited to, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1 10:1, 9:1, 8:1, 7:1, 6:1, 5:1, 4:1, 3:1, 2:1, 1:2, 1:3, 1:4, 1:5, 1:6, 1:7, 1:8, 1:9, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, and the like.

In some embodiments, a first polypeptide comprising a TTA domain can be purified from a cell at a close to 1:1 ratio (at approximately a 1:1: ratio) to a second polypeptide comprising the same TTA domain. The first polypeptide and the second polypeptide can include substantially similar domain linkers, cleavable linkers, half life extension domains, and any combination thereof.

In other embodiments, a first polypeptide comprising a TTA domain can be purified from a cell at a non-equimolar ratio to a second polypeptide comprising a different TTA domain. The TTA domain of the first polypeptide and the TTA domain of the second polypeptide can have different binding affinities. In some cases, the first polypeptide and the second polypeptide can include different domain linkers, cleavable linkers, half life extension domains, and any combination thereof.

A specific amount of the polynucleotide (or expression vector) encoding the polypeptide can be expressed in a cell to produce a desired amount of the polypeptide. In some embodiments, the amount of the first polynucleotide (or first expression vector) encoding the first polypeptide and the amount of the polynucleotide (or expression vector) encoding the second polypeptide that are introduced (e.g., transfected, electroporated, transduced, and the like) into the cell are the same. For instance, the first polynucleotide and the second polynucleotide can be introduced into a cell at a polynucleotide ratio of about 1:1. In other embodiments, the amount of the first polynucleotide (or first expression vector) encoding the first polypeptide and the amount of the second polynucleotide (or expression vector) encoding the second polypeptide that is introduced into the cell are different. For example, the first polynucleotide and the second polynucleotide can be introduced into a cell at a polynucleotide ratio such as, but not limited to, 8:2, 7:3, 6:4, 5:5, 4:6, 3:7, 50:1, 45:1, 40:1, 35:1, 30:1, 25:1, 20:1, 15:1 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:30, 1:35, 1:40, 1:45, 1:50, and the like.

The expression vectors for the polypeptides can include one or more components (e.g., promoters, regulatory elements, enhancers, and the like) that enable production of the polypeptides at a desired ratio by the cell. In some cases, the first expression vector of the first polypeptide comprises components that increase the expression level of the vector compared to the expression level of the second expression vector of the second polypeptide. In other cases, the second expression vector of the second polypeptide comprises components that increase the expression level of the vector compared to the expression level of the first expression vector of the first polypeptide. In certain cases, the first expression vector of the first polypeptide comprises components such that the expression level of the vector is the same as the expression level of the second expression vector of the second polypeptide.

In some cases, a nucleic acid described herein provides for production of bispecific conditionally effective proteins of the present disclosure, e.g., in a mammalian cell. A nucleotide sequence encoding the first and/or the second polypeptide of the present disclosure can be operably linked to a transcriptional control element, e.g., a promoter, and enhancer, etc.

Suitable promoter and enhancer elements are known in the art. For expression in a bacterial cell, suitable promoters include, but are not limited to, lad, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters.

A nucleic acid or nucleotide sequence encoding a protein, e.g., a prodrug construct described herein can be present in an expression vector and/or a cloning vector. Where a protein, e.g., a prodrug construct comprises two separate polypeptides, nucleotide sequences encoding the two polypeptides can be cloned in the same or separate vectors. An expression vector can include a selectable marker, an origin of replication, and other features that provide for replication and/or maintenance of the vector. Suitable expression vectors include, e.g., plasmids, viral vectors, and the like.

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

The present disclosure provides a mammalian cell that is modified to produce a protein, e.g., a prodrug construct of the present disclosure. A polynucleotide described herein can be introduced into a mammalian cell using any method known to one skilled in the art such as, but not limited to, transfection, electroporation, viral infection, and the like.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HEK293 cells, expi293 cells, HLHepG2 cells, Hut-78, Jurkat, HL-60, NK cell lines (e.g., NKL, NK92, and YTS), and the like.

In some embodiments, the host cell or stable host cell line is selected according to the amount of polypeptide produced and secreted by the cell. The host cell or stable host cell line can produce and secrete the prodrug composition described herein. In some instances, a suitable cell may produce an equimolar ratio (e.g., an about 1:1 ratio) of any one of the first polypeptides and any one of the second polypeptides described herein. In other embodiments, a suitable cell produces a non-equimolar ratio (e.g., a ratio that differs from 1:1) of any one of the first polypeptides and any one of the second polypeptides.

### VI. Useful Embodiments of the Invention

In some embodiments, the host cell or stable host cell line is selected according to the amount of polypeptide produced and secreted by the cell. The host cell or stable host cell line can produce and secrete the prodrug composition described herein. In some instances, a suitable cell may produce an equimolar ratio (e.g., an about 1:1 ratio) of any one of the first polypeptides and any one of the second polypeptides described herein. In other embodiments, a suitable cell produces a non-equimolar ratio (e.g., a ratio that differs from 1:1) of any one of the first polypeptides and any one of the second polypeptides.

In one aspect, provided herein is a cell comprising: (a) a first polynucleotide sequence encoding a first polypeptide comprising, from N- to C-terminal: (i) a first sdAb that binds to a human tumor target antigen (TTA); (ii) a first domain linker; (iii) a variable heavy chain comprising vhCDR1, vhCDR2, and vhCDR3; (iv) a first cleavable linker comprising a first protease cleavage site; and (v) a pseudo variable light chain; and (b) a second polynucleotide sequence encoding a second polypeptide comprising, from N- to C-terminal: (i) a second sdAb that binds to a human tumor target antigen (TTA); (ii) a second domain linker; (iii) a variable light chain comprising vlCDR1, vlCDR2, and vlCDR3; (iv) a second cleavable linker comprising a second protease cleavage site; and (v) a pseudo variable heavy chain. In some cases, the variable heavy chain of the first polypeptide and the variable light chain of the second polypeptide are capable of binding human CD3 when associated to form a Fv (e.g., a paired Vh-Vl).

In some embodiments, the variable heavy chain comprises vhFR1-vhCDR1-vhFR2-vhCDR2-vhFR3-vhCDR3-vhFR4. In certain embodiments, the variable light chain comprises vlFR1-vlCDR1-vlFR2-vlCDR2-vlFR3-vlCDR3-vlFR4. In particular embodiments, the pseudo variable heavy chain comprises vhFR1-vhiCDR1-vhFR2-vhiCDR2-vhFR3-vhiCDR3-vhFR4. In some embodiments, the pseudo variable light chain comprises vlFR1-vliCDR1-vlFR2-vliCDR2-vlFR3-vliCDR3-vlFR4.

In some embodiments, the first sdAb and the second sdAb bind to the same human TTA. In certain instances, the first sdAb and the second sdAb bind to the same human EGFR (e.g., the same EGFR molecule). In some embodiments, the first sdAb and the second sdAb comprise the same sequence. In certain embodiments, the first sdAb and the second sdAb comprise different sequences.

In some embodiments, the first and second protease cleavage sites are recognized by the same protease.

In various embodiments, the first polypeptide further comprises a half life extension domain at the C-terminal end. In other embodiments, the second polypeptide further comprises a half life extension domain at the C-terminal end.

In some embodiments, the first polypeptide is selected from the group consisting of Pro16 (SEQ ID NO:3), Pro39 (SEQ ID NO:5), Pro41 (SEQ ID NO:7), Pro43 (SEQ ID NO:9), Pro45 (SEQ ID NO:11), and Pro349 (SEQ ID NO:13). In some embodiments, the second polypeptide is selected from the group consisting of Pro19 (SEQ ID NO:4), Pro40 (SEQ ID NO:6), Pro42 (SEQ ID NO:8), Pro44 (SEQ ID NO:10), Pro46 (SEQ ID NO:12), and Pro353 (SEQ ID NO:14).

In some embodiments, the first polypeptide and the second polypeptide are selected from the group consisting of Pro16+Pro19, Pro39+Pro40, Pro41+Pro42, Pro43+Pro44, Pro45+Pro46, and Pro349+Pro353.

In some embodiments, the first polynucleotide sequence and the second polynucleotide sequence have been introduced into the cell on the same expression vector. In some embodiments, the first polynucleotide sequence and the second polynucleotide sequence have been introduced into the cell on different expression vectors.

In another aspect, provided herein is a method of isolating a prodrug composition comprising a first polypeptide and a second polypeptide. The method comprises (1) culturing a host cell under suitable conditions wherein a first polypeptide and a second polypeptide are produced and secreted into culture medial; and (2) purifying the first polypeptide and the second polypeptide from the culture media using Protein A chromatography, thereby isolating a prodrug composition comprising the first polypeptide and the second polypeptide. The host cell comprises (a) a first polynucleotide encoding the first polypeptide comprising, from N- to C-terminal: (i) a first sdAb that binds to a human tumor target antigen (TTA); (ii) a first domain linker; (iii) a variable heavy chain comprising vhCDR1, vhCDR2, and vhCDR3; (iv) a first cleavable linker comprising a first protease cleavage site; and (v) a pseudo variable light chain; and (b) a second polynucleotide encoding a second polypeptide comprising, from N- to C-terminal: (i) a second sdAb that binds to a human tumor target antigen (TTA); (ii) a second domain linker; (iii) a variable light chain comprising vlCDR1, vlCDR2, and vlCDR3; (iv) a second cleavable linker comprising a second protease cleavage site; and (v) a pseudo variable heavy chain; wherein the variable heavy chain of the first polypeptide and the variable light chain of the second polypeptide are capable of binding human CD3 when associated to form a Fv (e.g., a paired Vh-Vl).

In some embodiments, the first polypeptide and the second polypeptide are purified separately. In particular embodiments, the first polypeptide and the second polypeptide are purified simultaneously.

In some embodiments, the prodrug composition comprises an equimolar ratio of the first polypeptide and the second polypeptide. In other embodiments, the prodrug composition comprises a ratio of the first polypeptide and the second polypeptide that is not about 1:1.

In some embodiments, the purifying step further comprises performing affinity chromatography after the Protein A chromatography.

In some embodiments, the variable heavy chain comprises vhFR1-vhCDR1-vhFR2-vhCDR2-vhFR3-vhCDR3-vhFR4. In certain embodiments, the variable light chain comprises vlFR1-vlCDR1-vlFR2-vlCDR2-vlFR3-vlCDR3-vlFR4. In particular embodiments, the pseudo variable heavy chain comprises vhFR1-vhiCDR1-vhFR2-vhiCDR2-vhFR3-vhiCDR3-vhFR4. In some instances, the pseudo variable heavy chain comprises comprises a sequence selected from the group consisting of SEQ ID NO:106, SEQ ID NO:110, and SEQ ID NO:207. In some embodiments, the pseudo variable light chain comprises vlFR1-vliCDR1-vlFR2-vliCDR2-vlFR3-vliCDR3-vlFR4. In some instances, the pseudo variable light chain comprises comprises a sequence selected from the group consisting of SEQ ID NO:94, SEQ ID NO:98, and SEQ ID NO:203.

In some embodiments, the first sdAb and the second sdAb bind to the same human TTA. In certain instances, the first sdAb and the second sdAb bind to the same human EGFR.

In some embodiments, the first sdAb and the second sdAb comprise the same amino acid sequence (e.g., protein sequence). In some embodiments, the first sdAb and the second sdAb comprise different amino acid sequences.

In some embodiments, the first and second protease cleavage sites are recognized by the same protease.

In some embodiments, the first polypeptide further comprises a half life extension domain at the C-terminal end. In particular embodiments, the second polypeptide further comprises a half life extension domain at the C-terminal end.

In some embodiments, the first polypeptide is selected from the group consisting of Pro16 (SEQ ID NO:3), Pro39 (SEQ ID NO:5), Pro41 (SEQ ID NO:7), Pro43 (SEQ ID NO:9), Pro45 (SEQ ID NO:11), an dPro349 (SEQ ID NO:13). In some embodiments, the second polypeptide is selected from the group consisting of Pro19 (SEQ ID NO:4), Pro40 (SEQ ID NO:6), Pro42 (SEQ ID NO:8), Pro44 (SEQ ID NO:10), Pro46 (SEQ ID NO:12), and Pro353 (SEQ ID NO:14).

In some embodiments, the first polypeptide and the second polypeptide are selected from the group consisting of Pro16+Pro19, Pro39+Pro40, Pro41+Pro42, Pro43+Pro44, Pro45+Pro46, and Pro349+Pro353.

In some embodiments, the first polynucleotide sequence and the second polynucleotide sequence have been introduced into the cell on the same expression vector. In some embodiments, the first polynucleotide and the second polynucleotide have been introduced into the cell on different expression vectors.

In one aspect, provided herein is a polypeptide comprising (a) an amino acid sequence comprising, from N- to C-terminal: (i) a first sdAb that binds to a human tumor target antigen (TTA); (ii) a domain linker; (iii) a variable heavy chain comprising vhCDR1, vhCDR2, and vhCDR3; (iv) a first cleavable linker comprising a first protease cleavage site; and (v) a pseudo variable light chain; or (b) an amino acid sequence comprising, from N- to C-terminal: (i) a second sdAb that binds to a human tumor target antigen (TTA); (ii) a second domain linker; (iii) a variable light chain comprising vlCDR1, vlCDR2, and vlCDR3; (iv) a second cleavable linker comprising a second protease cleavage site; and (v) a pseudo variable heavy chain. In some embodiments, the variable heavy chain of the polypeptide and the variable light chain of the polypeptide are capable of binding human CD3 when associated to form a Fv.

In some embodiments, the first sdAb and the second sdAb bind to the same human TTA. In certain instances, the first sdAb and the second sdAb bind to the same human EGFR.

In some embodiments, the first sdAb and the second sdAb comprise the same amino acid sequence (e.g., protein sequence). In some embodiments, the first sdAb and the second sdAb comprise different amino acid sequences.

In some embodiments, the first and second protease cleavage sites are recognized by the same protease.

In some embodiments, the polypeptide comprising the variable heavy chain further comprises a half life extension domain at the C-terminal end. In various embodiments, the polypeptide comprising the variable light chain further comprises a half life extension domain at the C-terminal end.

In some embodiments, the polypeptide is Pro349 (SEQ ID NO:13). In other embodiments, the polypeptide is selected from the group consisting of Pro353 (SEQ ID NO:14). In some embodiments, Pro349 (SEQ ID NO:13) is paired with Pro353 (SEQ ID NO:14).

In some embodiments, provided herein is a polynucleotide sequence encoding any polypeptide comprising the variable heavy chain described herein, e.g., the variable heavy chain that can bind CD3. In certain embodiments, provided herein is a polynucleotide sequence encoding any polypeptide comprising the variable light chain described herein, e.g., the variable light chain that can bind CD3.

In some embodiments, provided herein is an expression vector comprising the polynucleotide sequence encoding any polypeptide comprising the variable heavy chain described herein, e.g., the variable heavy chain that can bind CD3. In other embodiments, provided herein is an expression vector comprising the polynucleotide sequence encoding any polypeptide comprising the variable light chain described herein, e.g., the variable light chain that can bind CD3. In some embodiments, an expression vector comprising the polynucleotide sequence encoding any polypeptide comprising the variable heavy chain described herein, e.g., the variable heavy chain that can associate with the variable light chain to bind CD3 and the polynucleotide sequence encoding any polypeptide comprising the variable light chain described herein, e.g., the variable light chain that can associate with the variable light chain to bind CD3. In particular embodiments, provided herein is a host cell comprising any of the expression vectors of the invention. In some instances, the host cell is a mammalian cell.

In some aspect, provided herein is a prodrug comprising a polypeptide comprising the variable heavy chain described herein and the polypeptide comprising the variable light chain described herein, wherein the variable heavy chain and the variable light chain are capable of binding human CD3 when associated to form a Fv (e.g., a paired Vh-Vl).

In another aspect, provided herein is a method of treating cancer in a human subject in need thereof comprising administering any prodrug composition described herein.

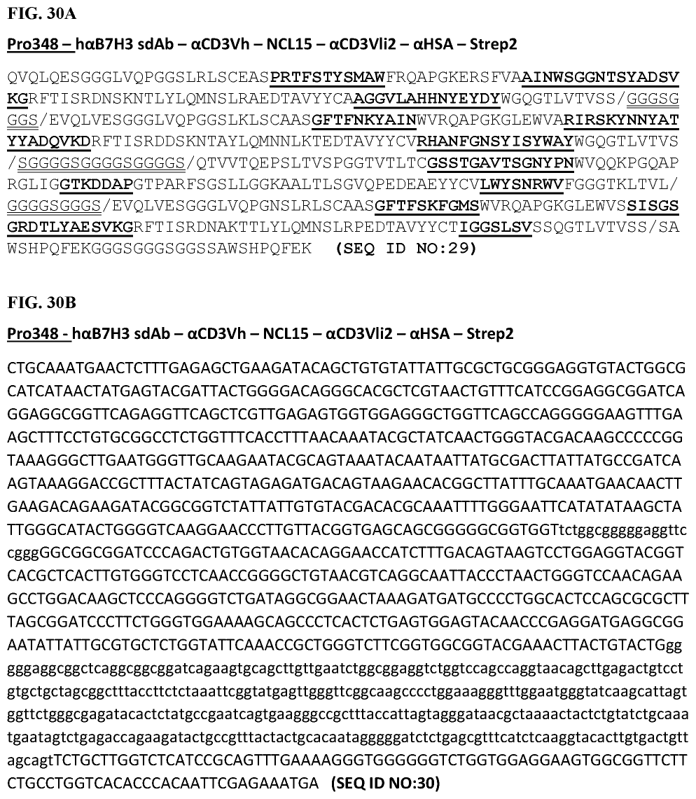

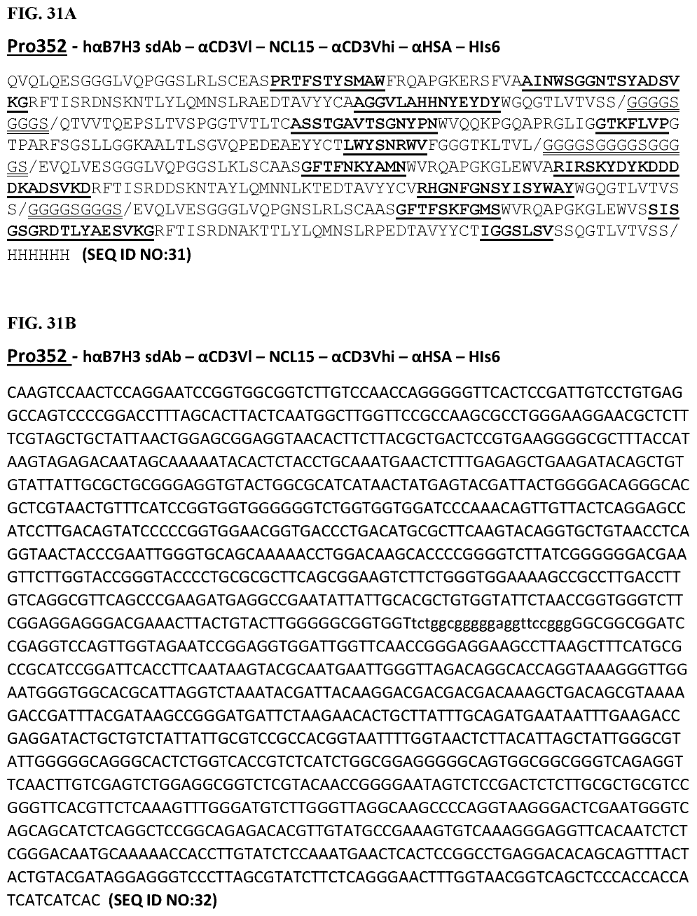

Another exemplary hemi-COBRA pair comprises a first polypeptide having the structure (N-terminus to C-terminus): sdAb(TTA)-DL-hFR1-vhCDR1-vhFR2-vhCDR2-vhFR3-vhCDR3-vhFR4-DL-vlFR1-vliCDR1-vlFR2-vliCDR2-vlFR3-vliCDR3-vlFR4, and a second polypeptide having the structure (N-terminus to C-terminus): sdAb(TTA)-DL-vlFR1-vlCDR1-vlFR2-vlCDR2-vlFR3-vlCDR3-vlFR4-DL-vhFR1-vhiCDR1-vhFR2-vhiCDR2-vhFR3-vhiCDR3-vhFR4. In some embodiments, the pair comprises Pro348/Pro352. In some embodiments, the pair comprises Pro350/Pro354. In some embodiments of the invention, the isolated cell expresses (produces or secretes) Pro348 and Pro352. In certain embodiments, the isolated cell expresses (produces or secretes) Pro350 and Pro354. In some embodiments, the host cell comprises an expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more identity to SEQ ID NO:29, as shown in FIG. 30A and/or a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity to SEQ ID NO:31, as shown in FIG. 31A. In some embodiments, the host cell comprises an expression vector comprising a first polynucleotide encoding a first polypeptide comprising a structure shown in FIG. 30A and/or a second polynucleotide encoding a second polypeptide comprising a structure shown in FIG. 31A. In certain embodiments, the expression vector comprises a first nucleic acid sequence shown in FIG. 30B and/or a second nucleic acid sequence shown in FIG. 31B. In some embodiments, the expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% sequence identity, or more to SEQ ID NO:30 and/or a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% sequence identity, to SEQ ID NO:32. In some embodiments, the expression vector comprises a nucleic acid sequence encoding Pro348 such as SEQ ID NO:30 and/or a nucleic acid sequence encoding Pro352 such as SEQ ID NO:32.

In some embodiments, the host cell comprises a first expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity to SEQ ID NO:29 as shown in FIG. 30A and a second expression vector comprising a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity to SEQ ID NO:31, as shown in FIG. 31A. In some embodiments, the host cell comprises a first expression vector comprising a first polynucleotide encoding a first polypeptide comprising a structure shown in FIG. 30A and a second expression vector comprising a second polynucleotide encoding a second polypeptide comprising a structure shown in FIG. 31A. In certain embodiments, the first expression vector comprises a first nucleic acid sequence shown in FIG. 30B and the first expression vector comprises a second nucleic acid sequence shown in FIG. 31B. In some embodiments, the first expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% sequence identity, or more to SEQ ID NO:30 and the second expression vector comprises a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% sequence identity, to SEQ ID NO:32. In some embodiments, the first expression vector comprises a nucleic acid sequence encoding Pro348 such as SEQ ID NO:30 and the second expression vector comprises nucleic acid sequence encoding Pro352 such as SEQ ID NO:32.

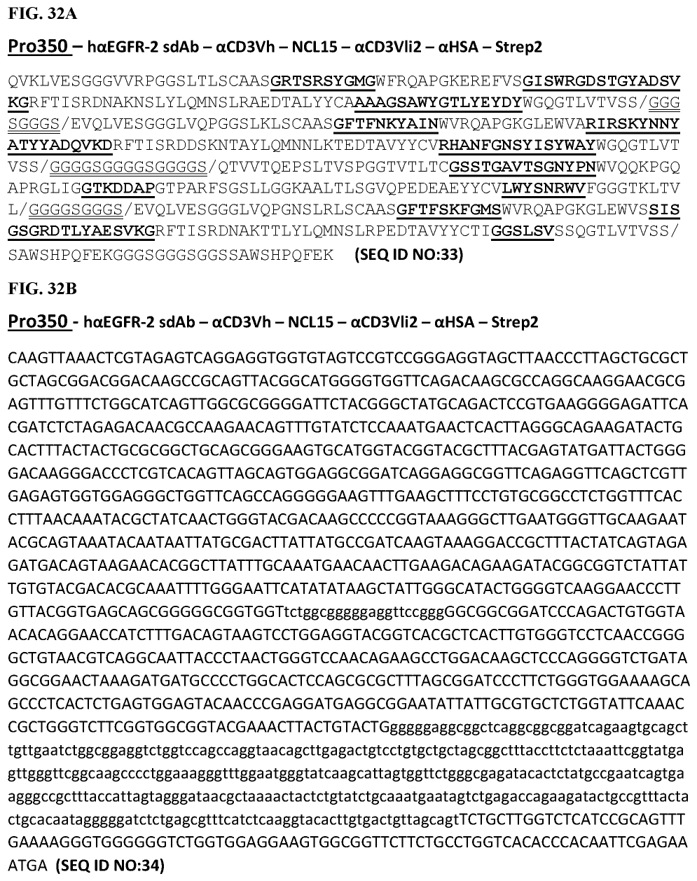

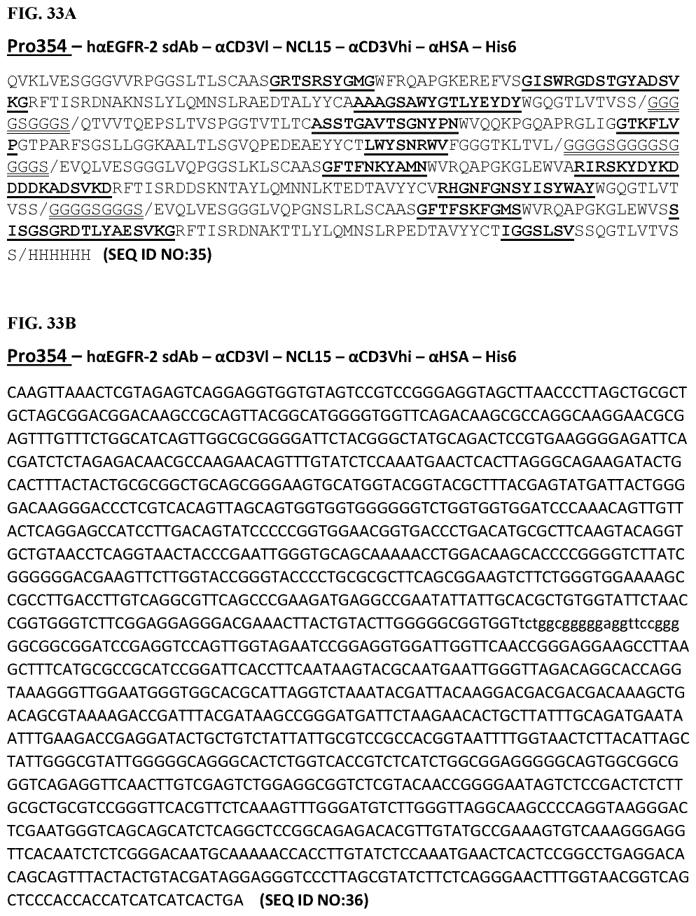

In some embodiments, the host cell comprises an expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity to SEQ ID NO:33, as shown in FIG. 32A and/or a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity to SEQ ID NO:35, as shown in FIG. 33A. In some embodiments, the host cell comprises an expression vector comprising a first polynucleotide encoding a first polypeptide comprising a structure shown in FIG. 32A and/or a second polynucleotide encoding a second polypeptide comprising a structure shown in FIG. 33A. In certain embodiments, the expression vector comprises a first nucleic acid sequence shown in FIG. 32B and/or a second nucleic acid sequence shown in FIG. 33B. In some embodiments, the expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% sequence identity, or more to SEQ ID NO:34 and/or a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% sequence identity, to SEQ ID NO:36. In some embodiments, the expression vector comprises a nucleic acid sequence encoding Pro350 such as SEQ ID NO:33 and/or a nucleic acid sequence encoding Pro354 such as SEQ ID NO:36.

In some embodiments, the host cell comprises a first expression vector comprising a first polynucleotide encoding a first polypeptide comprising an amino acid sequence having at least 90% sequence identity, e.g., 90%, 91%, 92%, 93%, 94%, 95%, 96%, 97%, 98%, 99% or more sequence identity to SEQ ID NO:33 as shown in FIG. 32A and a second expression vector comprising a second polynucleotide encoding a second polypeptide comprising an amino acid sequence having at least 90% sequence identity, e.g., 90%, 91%, 92%, 93%, 94%, 95%, 96%, 97%, 98%, 99% or more sequence identity to SEQ ID NO:35, as shown in FIG. 33A. In some embodiments, the host cell comprises a first expression vector comprising a first polynucleotide encoding a first polypeptide comprising a structure shown in FIG. 32A and a second expression vector comprising a second polynucleotide encoding a second polypeptide comprising a structure shown in FIG. 33A. In certain embodiments, the first expression vector comprises a first nucleic acid sequence shown in FIG. 32B and the first expression vector comprises a second nucleic acid sequence shown in FIG. 33B. In some embodiments, the first expression vector comprises a first nucleic acid sequence having at least 85% sequence identity, e.g., 85%, 86%, 87%, 88%, 89%, 89%, 90%, 91%, 92%, 93%, 94%, 95%, 96%, 97%, 98%, 99% sequence identity, or more to SEQ ID NO:34 and the second expression vector comprises a second nucleic acid sequence having at least 85% sequence identity, e.g., 85%, 86%, 87%, 88%, 89%, 89%, 90%, 91%, 92%, 93%, 94%, 95%, 96%, 97%, 98%, 99% sequence identity, to SEQ ID NO:36. In some embodiments, the first expression vector comprises a nucleic acid sequence encoding Pro350 such as SEQ ID NO:34 and the second expression vector comprises nucleic acid sequence encoding Pro354 such as SEQ ID NO:36.

In some embodiments, the polynucleotide ratio transfected into a host cell described herein is 2:8, 3:7, 4:6, 5:5, 6:4, and 7:3 of the first polynucleotide encoding a first polypeptide and the second polynucleotide encoding a second polypeptide, respectively, wherein the first polypeptide comprises an amino acid sequence having at least 90% sequence identity, e.g., 90%, 91%, 92%, 93%, 94%, 95%, 96%, 97%, 98%, 99% or more sequence identity, to SEQ ID NO:29 and the second polypeptide comprises an amino acid sequence having at least 90% sequence identity, e.g., 90%, 91%, 92%, 93%, 94%, 95%, 96%, 97%, 98%, 99% or more sequence identity, to SEQ ID NO:31. In some embodiments, the polynucleotide ratio is 2:8, 3:7, 4:6, 5:5, 6:4, and 7:3 of the first polynucleotide encoding a first polypeptide and the second polynucleotide encoding a second polypeptide, respectively, wherein the first polynucleotide comprises a nucleic acid sequence having at least 90% sequence identity to SEQ ID NO:30 and the second polynucleotide comprises a nucleic acid sequence having at least 90% sequence identity to SEQ ID NO:32. In other embodiments, the polynucleotide ratio transfected into a host cell described herein is 2:8, 3:7, 4:6, 5:5, 6:4, and 7:3 of the first polynucleotide encoding a first polypeptide and the second polynucleotide encoding a second polypeptide, respectively, wherein the first polypeptide comprises an amino acid sequence having at least 90% sequence identity, e.g., 90%, 91%, 92%, 93%, 94%, 95%, 96%, 97%, 98%, 99% or more sequence identity, to SEQ ID NO:33 and the second polypeptide comprises an amino acid sequence having at least 90% sequence identity, e.g., 90%, 91%, 92%, 93%, 94%, 95%, 96%, 97%, 98%, 99% or more sequence identity, to SEQ ID NO:35. In some embodiments, the polynucleotide ratio is 2:8, 3:7, 4:6, 5:5, 6:4, and 7:3 of the first polynucleotide encoding a first polypeptide and the second polynucleotide encoding a second polypeptide, respectively, wherein the first polynucleotide comprises a nucleic acid sequence having at least 85% sequence identity to SEQ ID NO:34 and the second polynucleotide comprises a nucleic acid sequence having at least 85% sequence identity to SEQ ID NO:36.

### VII. Examples

### A. Example 1: Method for Producing Hemi-COBRA Pairs

Hemi-COBRAs cannot induce T-cell killing as individual molecules even after protease activation, however, two activated, complementary hemi-COBRA molecules can induce potent T-cell mediated cytotoxicity against target expressing cells (FIG. 1 and FIG. 2). This creates a problem for producing these molecules as drugs, since two protein molecules need to be produced. One solution is to make two expression cell lines, each expressing a different hemi-COBRA, then the two hemi-COBRAs can be expressed and purified separately and mixed to produce a pro-drug cocktail. Unfortunately, this is a time-consuming and expensive process to generate large doses of the drug cocktail.

A solution to this problem is to co-express the two hemi-COBRAs in the same cell and then purify them either separately or together from the same conditioned media.

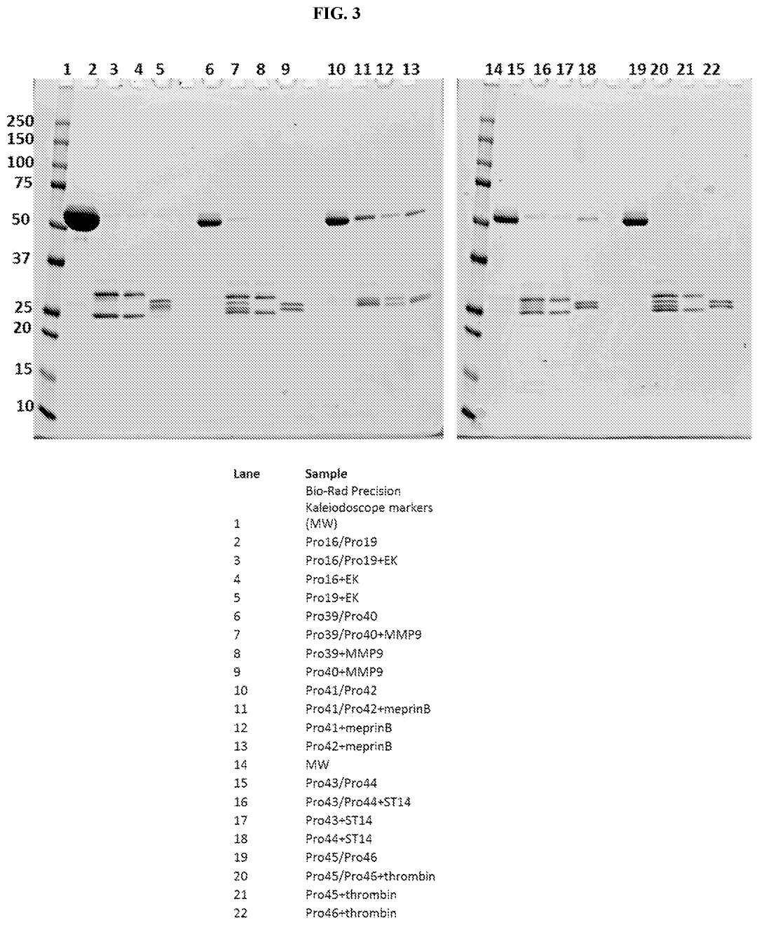

FIG. 3 shows that hemi-COBRAs were co-expressed from a transiently co-transfected population of expi293 cells, and then copurified from the resulting conditioned media using a Protein A purification column.

For the complementary pairs Pro39+Pro40, Pro41+Pro42 and Pro45+Pro46, the co-expression and co-purification method produced a roughly equimolar mixture of the two hemi-COBRAs. For the complementary pairs Pro16+Pro19 and Pro43+Pro44, one of the two hemi-COBRAs was more abundant in the purified product.

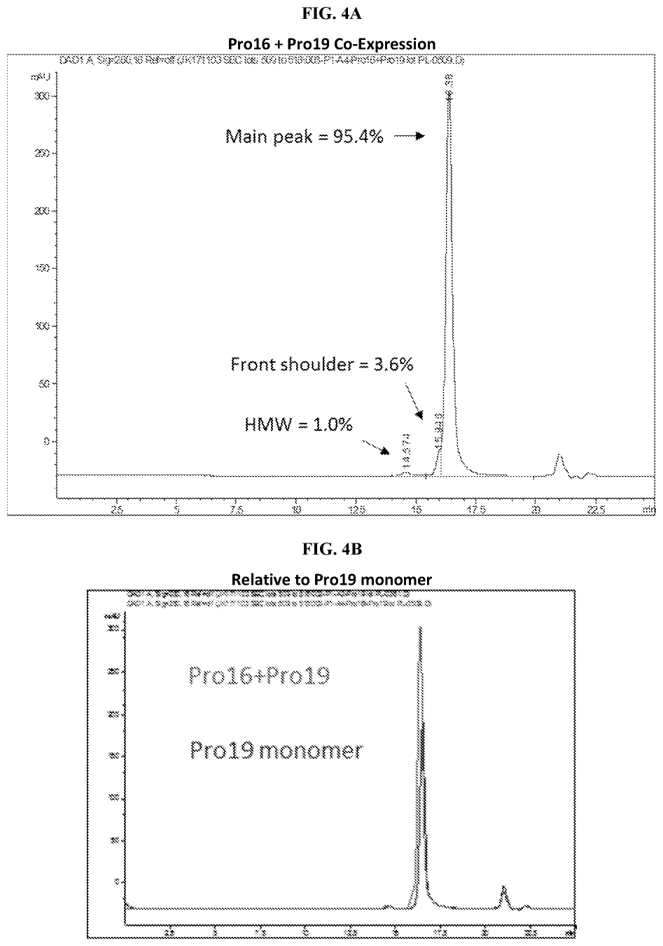

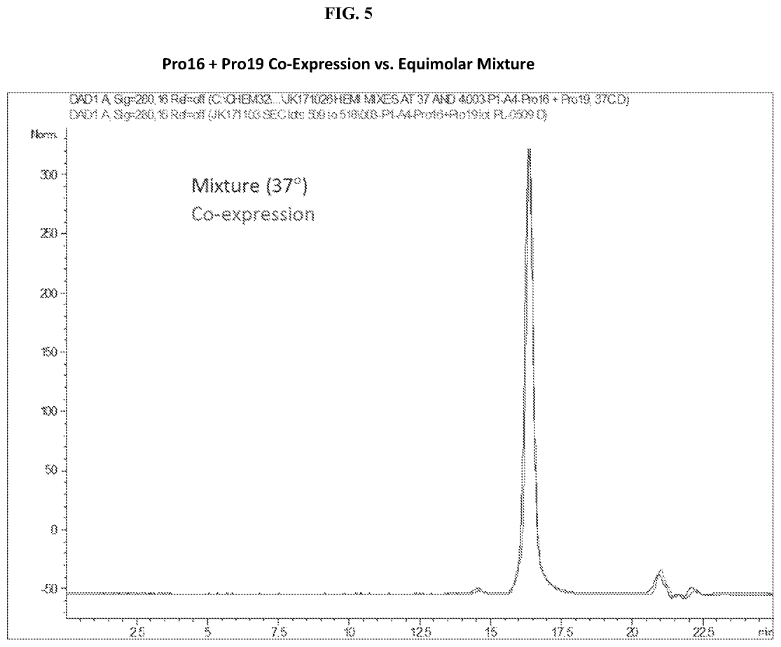

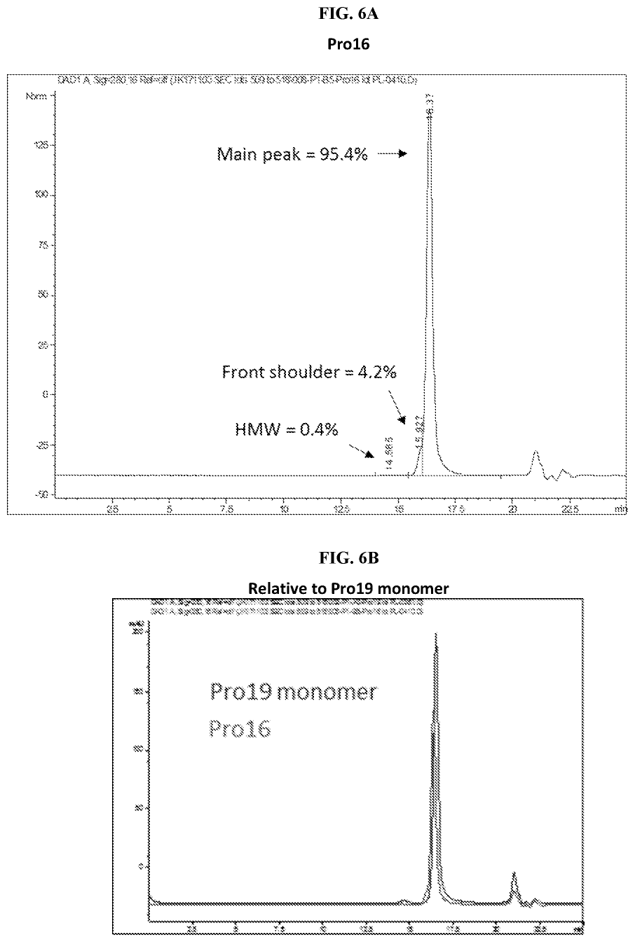

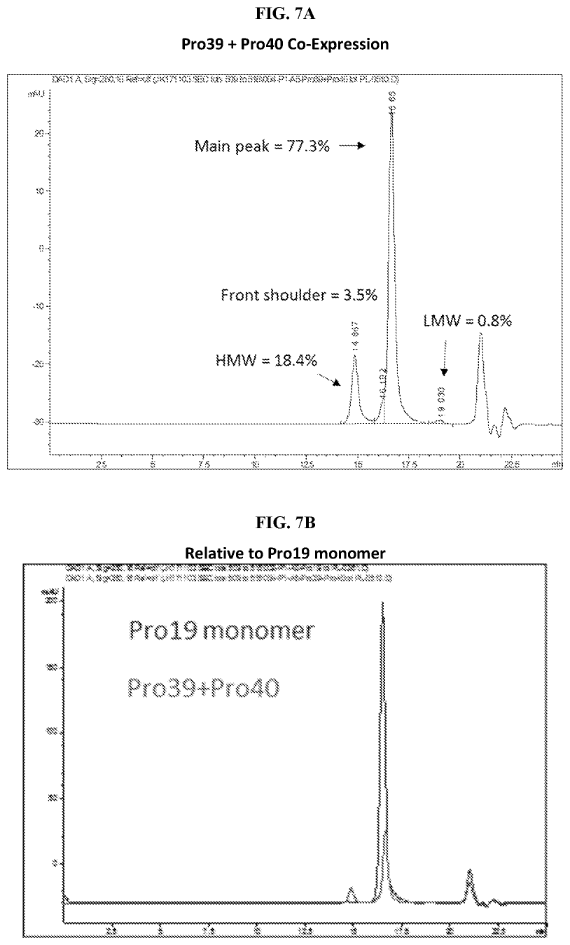

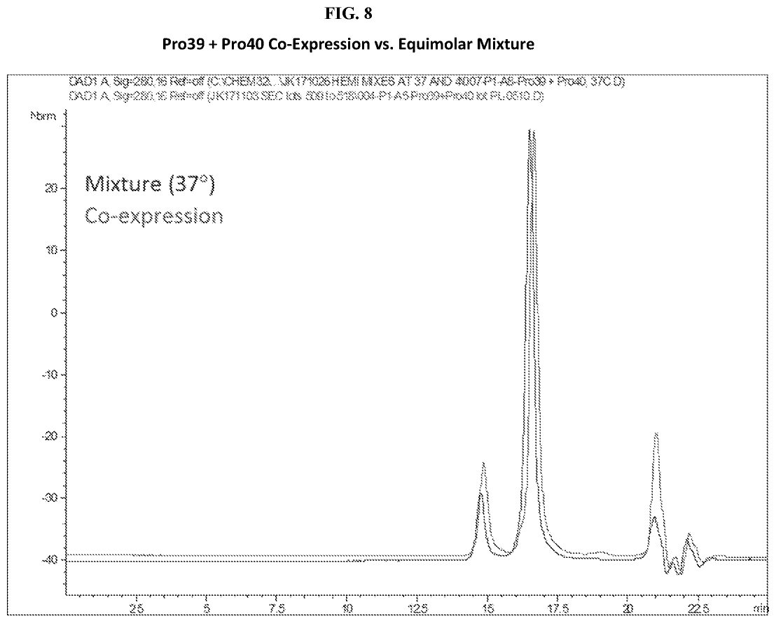

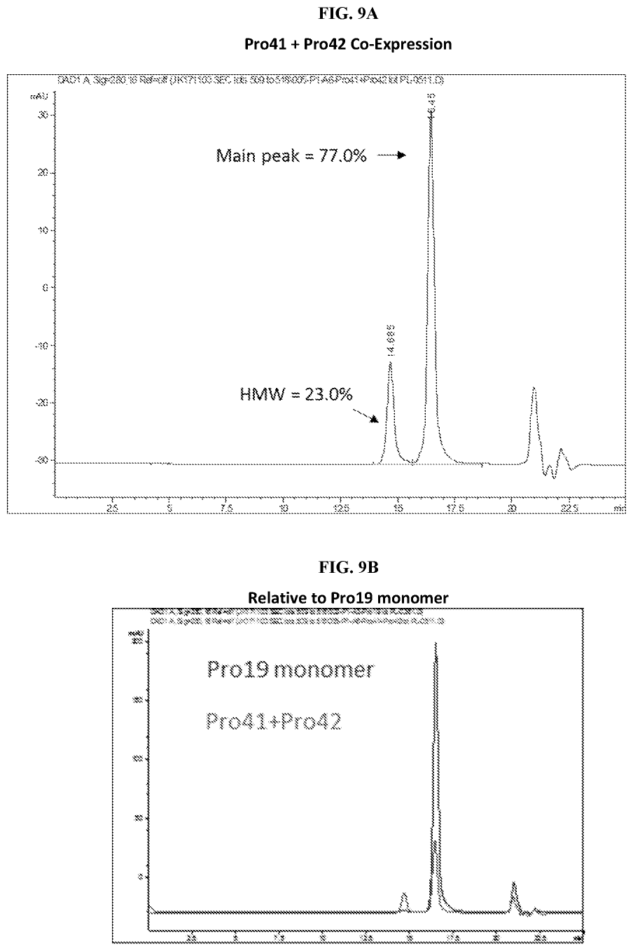

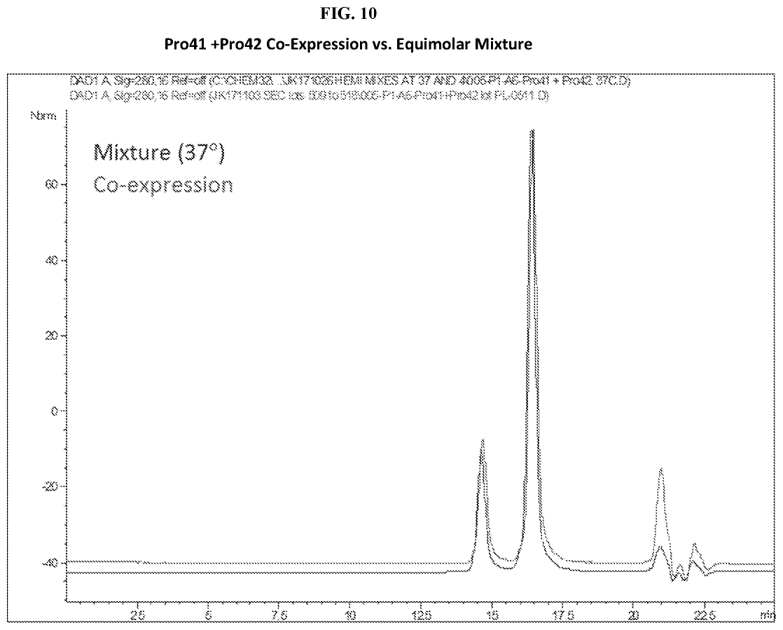

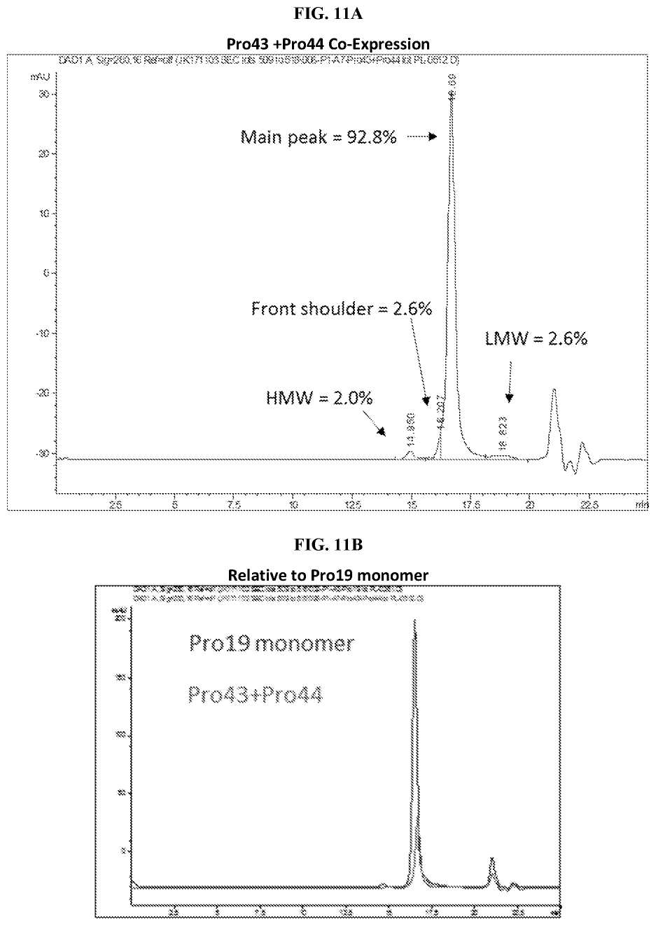

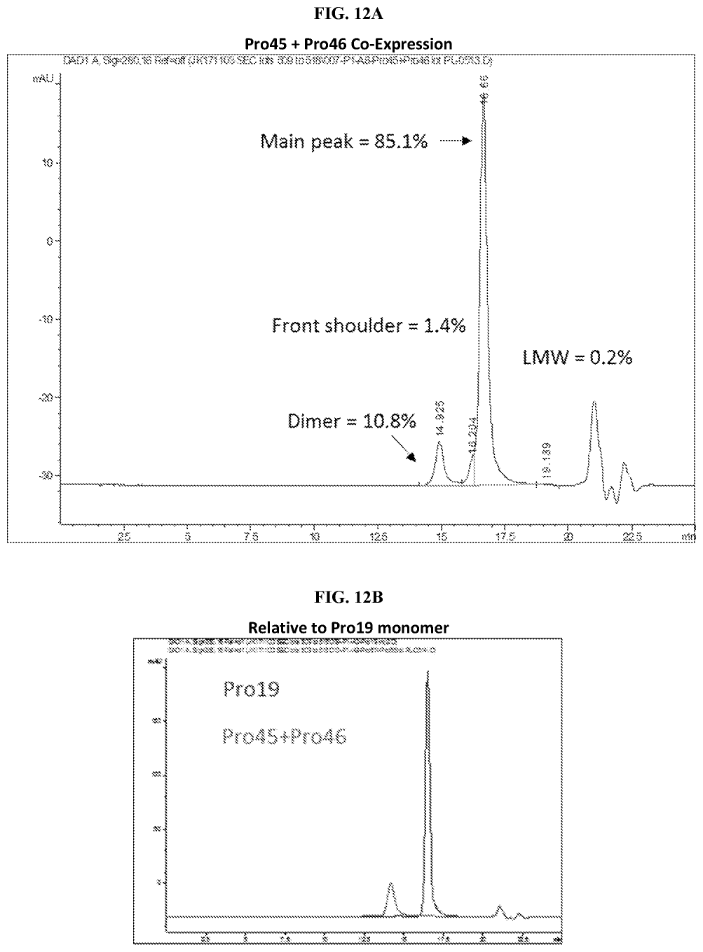

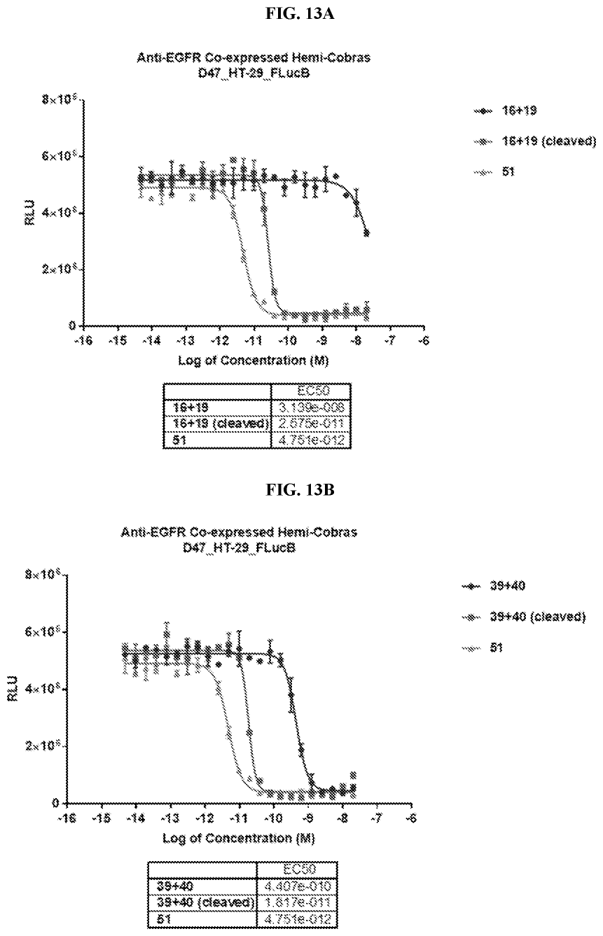

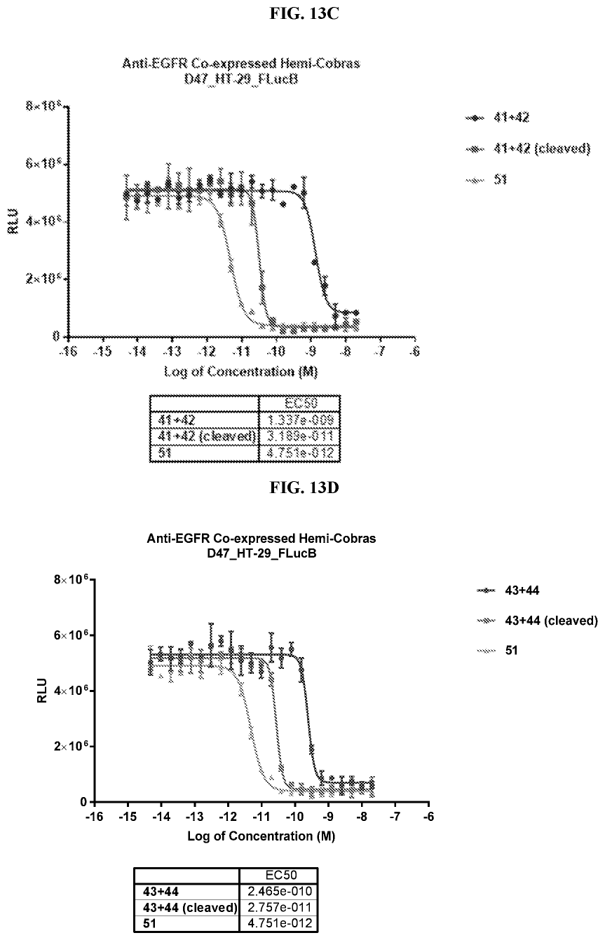

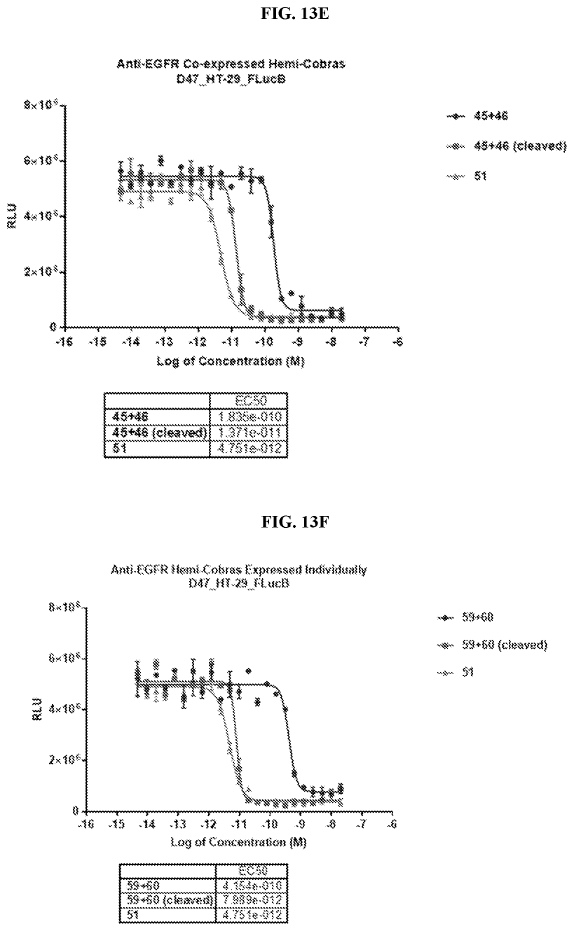

FIG. 4A-FIG. 12B show that the main product of these co-expression/purification lots as measured by size exclusion chromatography is equivalent to a monomeric hemi-COBRA. The results shows that co-expression and purification did not lead to protein aggregation. In T-cell killing assays (FIG. 13A-FIG. 13F) the co-expressed hemi-COBRAs show expected levels of potency given the concentration of lower expressed hemi-COBRA for each pair. The co-expressed hemi-COBRAs also showed good protease activation.

Complementary hemi-COBRA pairs can be produced in stable cell lines, e.g., CHO and 293 cells via co-transfection, co-expression from a single vector or retroviral co-transduction of two hemi-COBRA expression units. Stable clones that express high levels of both proteins with close to equimolar ratios can be selected. The data provided herein demonstrate that proteins of the invention can be co-expressed in a cell and then co-purified using a Protein A column.

In some embodiments, complementary hemi-COBRA pairs, such as but not limited to, Pro16+Pro19, Pro39+Pro40, Pro41+Pro42, Pro43+Pro44, Pro45+Pro46, and Pro349+Pro353, can be further purified based on differential carboxy-terminal tags on each monomer to yield pure lots of each hemi-COBRA. For instance, eluates from the Protein A column can be further purified though their differential carboxy-terminal tags by using affinity chromatography. The purified samples can be combined to yield equimolar mixtures, if desired.

A goal of the experiment described herein was to test whether co-expressed hemi-COBRAs produced two separate proteins. The intact hemi-COBRAs had the same molecular weight and could not be distinguished by SDS PAGE. But after the cleavage of the protease sensitive linker, the hemi-COBRAs with VH-VLi configuration and hemi-COBRAs with VL-VHi configuration produce digestion products with different molecular weights. Therefore, the proteolytic reactions were set up with the co-expressed hemi-COBRAs and individual hemi-COBRAs and then the products of proteolysis were visualized by SDS PAGE (FIG. 3).

The concentration of the samples was adjusted to 0.2 mg/ml in 100 μl volume using HBS+10 mM CaCl₂ buffer. CaCl₂ was added to each sample to the final concentration of 10 mM.

The following cleaving proteases were evaluated: enterokinase (New England Biolabs, #P8070S); Matriptase ST14 (R&D Systems, #3946-SE-010), used at 5.7 μM; Thrombin (Enzo, BML-SE363-1000) diluted to 100 nM; MMP9 (R&D Systems #911-MP-010), activated according to the manufacturer protocol and diluted to 100 nM; and Meprin 1B from cynomolgus monkeys, used at 135 nM, expressed and purified in-house and activated according to R&D Systems protocol for Meprin 1A (R&D Systems #3220-ZN-010).

Proteolytic reactions were set up as follows:

Pro16/19, Pro16, Pro19 were cleaved with enterokinase.

Pro39/40, Pro39, Pro40 were cleaved with MMP9.

Pro41/42, Pro41, Pro42 were cleaved with meprin 1B.

Pro43/44, Pro43, Pro44 were cleaved with matriptase ST14.

Pro45/46, Pro45, Pro46 were cleaved with thrombin.

The reactions were incubated overnight at room temperature. The samples were analyzed by SDS PAGE (non-reducing conditions) using the NuPAGE TG 10-20% gel in Tris-Glycine running buffer. Gels were run at 200 V for 1 hr.

From SDS PAGE analysis (FIG. 3), it was observed that hemi-COBRAs were co-expressed with some degree of variability of the molar ratio, with this value being close to 1:1 in Pro39/40, Pro41/42 and Pro45/46 co-expressed hemi-COBRAs.

Products from the co-expression and co-purification were analyzed using analytical size exclusion chromatography (SEC). The following parameters were used.

Instrument: Agilent 1100 HPLC with diode array detector

Column: Zenix-C SEC-300 column, 7.8×300 mm, 3 μm particle size, 300 Å pore size, Sepax Technologies, Newark, Del.

Mobile phase: 0.2 M L-arginine.HCl+0.1 M sodium phosphate, pH 7.0

Flow rate: 0.5 ml/min isocratic

Column temperature: 30° C.

Detection: absorbance at 280 nm

Sample preparation: none (inject neat)

Sample volume: 25 to 50 μL

The results of the SEC analysis are provided in FIG. 4A-FIG. 12.

The activity of co-expressed hemi-COBRAs was assessed using a T cell killing assay (T cell-dependent cellular cytotoxicity assay or TDCC). The results showed that the co-expressed hemi-COBRA pairs exhibited expected levels of potency consistent with the lower expressed hemi-COBRA for each pair. FIG. 13A depicts results of the cleaved Pro16 and Pro19 pair. FIG. 13B depicts results of the cleaved Pro39 and Pro40 pair. FIG. 13C depicts results of the cleaved Pro41 and Pro42 pair. FIG. 13D depicts results of the cleaved Pro43 and Pro44 pair. FIG. 13E depicts results of the cleaved Pro45 and Pro46 pair. FIG. 13F depicts results of the cleaved Pro59 and Pro60 pair. Pro51 represents a positive control for T cell dependent cytotoxicity.

### B. Example 2: Generation of Stable Cell Lines Co-Expressing Hemi-COBRA Pairs

This example illustrates an exemplary method for generating cell lines that co-express a pair of complementary hemi-COBRAs.

### Material and Methods

All cell culture media, transfections were from Life Technologies. The transfection reagents included the Expi293 transfection kit. The growth and expression medium used in the study was Expi293 Expression medium. The selection medium included DMEM+10% FBS+0.5 mg/ml of G418. All the biosensors for Octet analysis were from ForteBio including SAX (High Precision Streptavidin) and His1K (Anti-Penta-His).

### Results

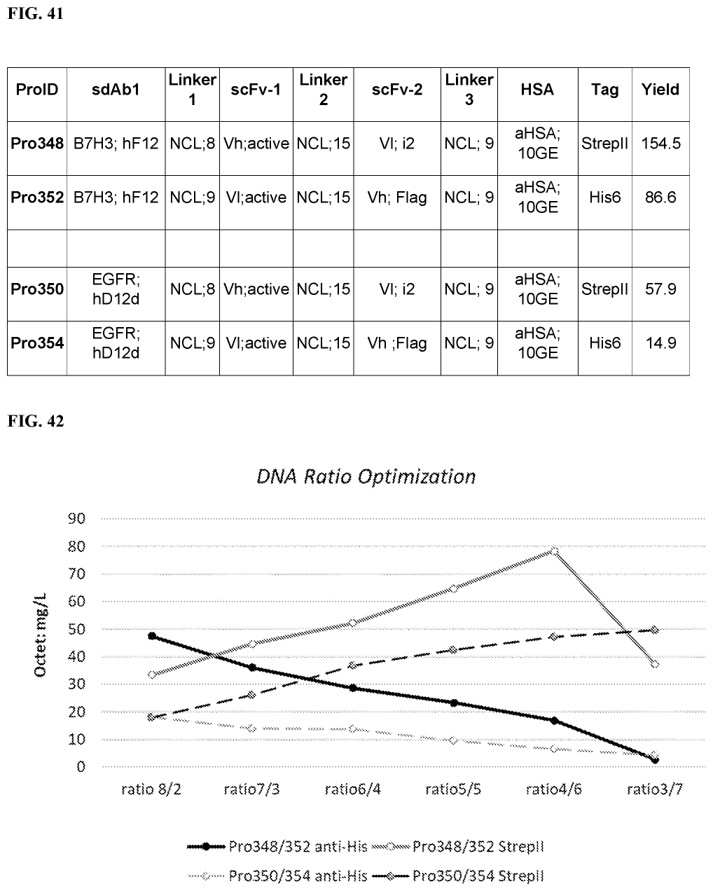

Schematics of the two pairs of hemi-COBRAs used for generating co-expressing stable cell lines are shown in FIG. 41. The hemi-COBRA pairs were tagged differently for easy detection and purification. Each test pair includes a first polypeptide having the structure (N-terminus to C-terminus): sdAb(TTA)-DL-hFR1-vhCDR1-vhFR2-vhCDR2-vhFR3-vhCDR3-vhFR4-DL-vlFR1-vliCDR1-vlFR2-vliCDR2-vlFR3-vliCDR3-vlFR4, and a second polypeptide having the structure (N-terminus to C-terminus): sdAb(TTA)-DL-vlFR1-vlCDR1-vlFR2-vlCDR2-vlFR3-vlCDR3-vlFR4-DL-vhFR1-vhiCDR1-vhFR2-vhiCDR2-vhFR3-vhiCDR3-vhFR4. The hemi-COBRA pairs tested were Pro348+Pro352 and Pro350+Pro354.

Pro348 which is represented by SEQ ID NO:29 includes an sdAb that binds B7H3, an anti-HSA domain, and a StrepII tag. Pro352 which is represented by SEQ ID NO:31 includes an sdAb that binds B7H3, an anti-HSA domain, and a His6 tag.

Pro350 which is represented by SEQ ID NO:33 includes an sdAb that binds EGFR, an anti-HSA domain, and a StrepII tag. Pro354 which is represented by SEQ ID NO:35 includes an sdAb that binds EGFR, an anti-HSA domain, and a His6 tag.

The table in FIG. 41 shows the yield of each hemi-COBRA when it was produced from Expi293 cells via transient transfection. In other words, each hemi-COBRA (Pro348, Pro352, Pro350, and Pro354) was separately transfected into Expi293 cells. The different productivities of each hemi-COBRA suggested that plasmid DNA concentration used for co-transfection should be optimized.

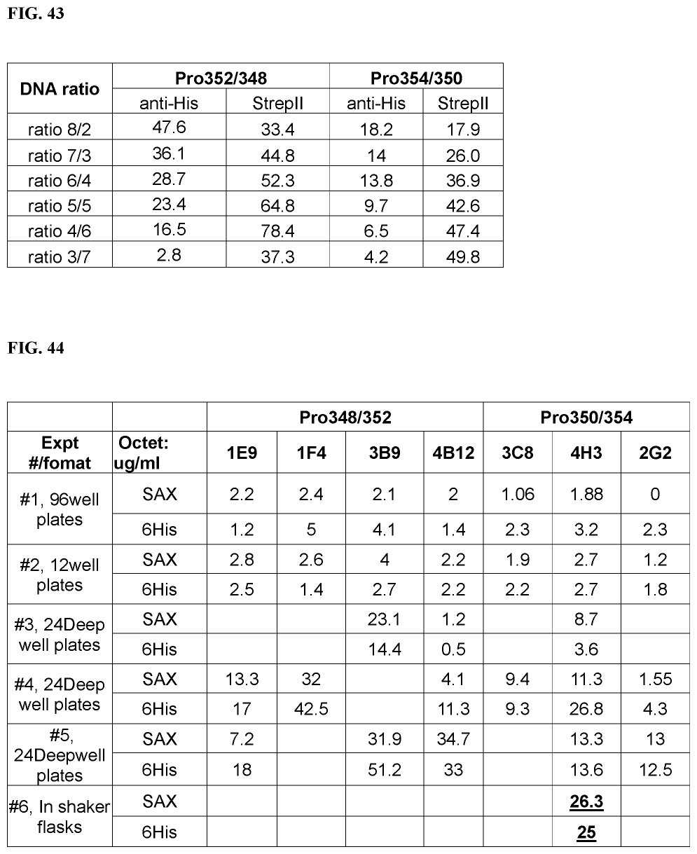

FIG. 42 and FIG. 43 show data of the plasmid DNA ratio optimization experiments. The goal was to identify the optimal DNA ratio for each pair that would result a similar productivity of the co-expressed hemi-COBRAs.

2.5 ml/well of Expi293 cells at VCD 2.9 were inoculated into 24 deep well plate in Expi293 medium. Expi293 transfection kit was used for transfection following the manufacturer's protocol. The plasmid DNA ratios tested for co-transfection were at 2:8; 3:7; 4:6; 5:5; 6:4; and 7:3. All transfections were duplicated. Enhancers were added 20 hours after transfection. 5 days after transfection, the conditioned media were collected for Octet analysis: SAX biosensor for Pro348 and Pro350, and His1K (anti-6His) for Pro352 and Pro354.

The optimal ratio for Pro348 and 352 was 2.5:7.5. The optimal ratio for Pro350 and 354 as 2:8. Briefly, plasmid pairs of Pro348/Pro352 were transfected to Expi293 cells using the Expi293 transfection kit. G418 selection was started 48 hours after transfection in 6 well plates. It took two weeks to complete the selection. When selection was completed, the cells were sorted into 96 w plates for stable cell line generation. Colonies were screened with Octet (anti-His and SAX). The top clones with similar expression levels of both hemi-COBRAs of the pair were picked for further testing. Three of final clones for each pair were identified, and banked. The expression levels of the co-transfected hemi-COBRAs were checked at each stage: transient, stable pools, and stable cell lines at different scales (see FIG. 44.)

Stable cell lines were generated according to the following method. For cell culturing, Expi293 cells were maintained in Expi293 medium with VCD of 0.3-5×10E6/ml in shaker flasks in a humidified incubator. The culture condition was 37° C., 8% CO₂ with a shake speed of 225 rpm. The same culture condition was used for the cells after transfection, however, the shaking speed varied depending on the flask/plate size.

Plasmid DNA pairs of Pro348/Pro352 (DNA ratio at 2.5:7.5) and Pro350/Pro354 (DNA ratio 2:8) were co-transfected into Expi293 cells using the Expi293 transfection kit following the manufacturer's protocol. Briefly, cells were inoculated at 2.9×10E6/ml×2.5 ml per well in 24-deep-well plate with rpm of 350 on a MixMate (Eppendorf) shaker. DNA/Expi293fectamine/cells ratio were at 1 μg/3 μl/1 ml. Plasmid DNAs and Expi293fectamin were diluted in OptiMEM at 1/20^{th} of the final transfection volume separately, then combined, and incubated at room temperature for 20′ before adding to the cells. Cells were then put back into the incubator at the standard culture condition described above.

For generating stable cell lines, two days after transfection, 2×10E6 transfected cells were seeded into T75 with selection medium. After recovering from selection, the cells were sorted into 96-well plates at 1 cell/well. The remaining cells were adapted to suspension for stable pools. The surviving clones were screened for productivities using 2 sets of Octet/Biosensors: SAX for Pro348 and Pro350, and His1K for Pro352 and Pro354. Cell confluency (IncuCyte) was used for normalization. The top clones with similar productivities of the co-expressed hemi-COBRAs were picked and transferred to 12-well plates.

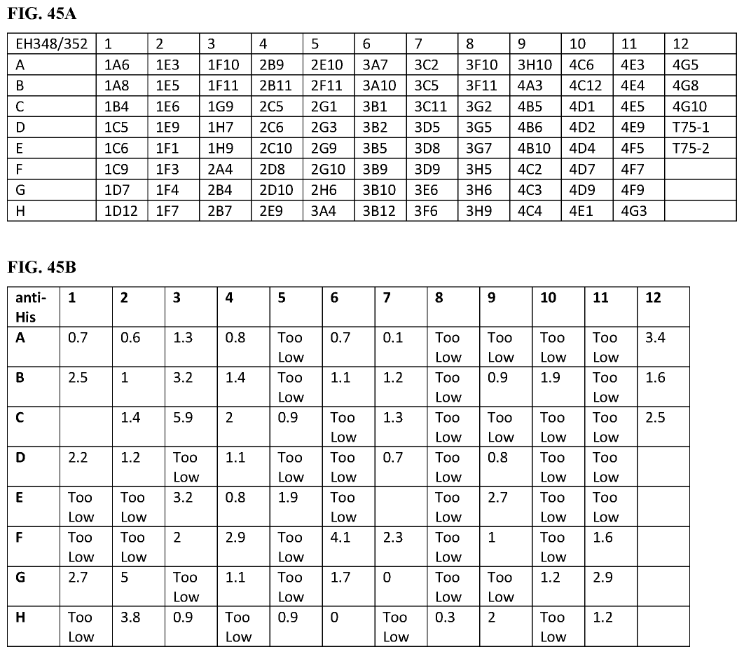

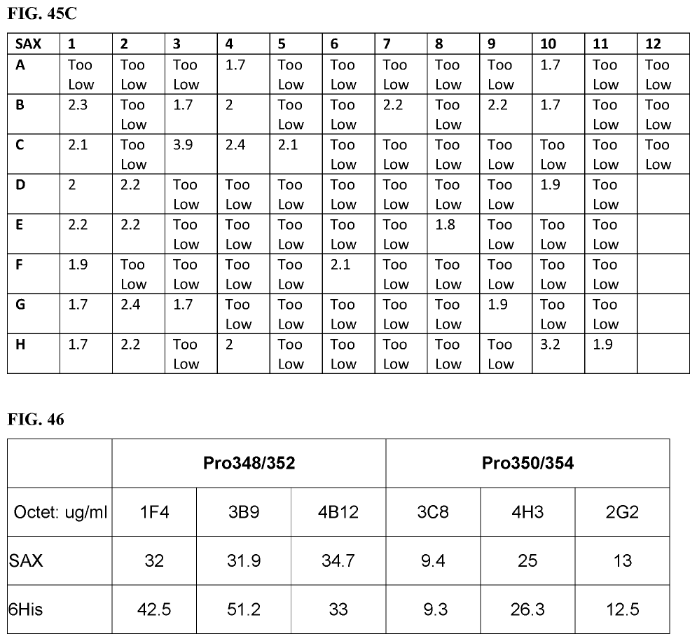

For colony screening, cells that survived selection were plated in 96 well plates at one cell per well. The conditioned media from recovered colonies was transferred to 2 sets of 96 well plates for Octet analysis. FIG. 45A-FIG. 45C depict exemplary data from the colony screening. FIG. 45A provides clone IDs. FIG. 45B shows data using His1K biosensors to detect His-tagged Pro352 and 354. FIG. 45B shows data using SAX to detect StrepII tagged Pro348 and Pro350.

To adapt the cells to suspension culture, when the cells reached 90% confluency, the selection medium was changed to Expi293 medium plus 0.5 mg/ml of G418. One day after medium changing, the cells were transferred to 24-deep well plates with the same medium and put on MixMate with rpm 350. The productivities of the co-expressed hemi-COBRAs were monitored at every stage of expansion.

The conditioned media was collected from 24 deep well plates and analyzed using Octet. FIG. 46 shows data from the analysis of the 3 top clones of both cell lines.

After adaptation to suspension culture, the cells were inoculated at 0.5×10E6/ml. 3 days after inoculation, the cells were feed with 5% of Efficient Feed B. Conditioned media were collected 4 days after feeding for Octet analysis to obtain a volumetric productivity estimation.

In summary, this example describes an exemplary method for producing a hemi-COBRA pair such that the hemi-COBRAs are expressed at substantially equivalent amounts. In this study, the plasmid DNA ratio of each hemi-COBRA transfected into a host cell was not 1:1. Instead, more plasmid DNA encoding one hemi-COBRA was transfected compared to the plasmid DNA encoding the second hemi-COBRA.

Additional detailed descriptions of the methods, protocols, and assays used can be found in WO 2019/051122, filed on Sep. 6, 2018, U.S. Provisional Application No. 62/555,999, filed on Sep. 8, 2017, WO 2019/051102, filed on Sep. 6, 2018, U.S. Provisional Application No. 62/555,943, filed on Sep. 8, 2017, U.S. Provisional Application No. 62/586,627, filed on Nov. 15, 2017, U.S. Provisional Application No. 62/587,318, filed on Nov. 16, 2017, WO2017/156178, filed on Mar. 8, 2017, U.S. Provisional Application No. 62/555,999, filed on Sep. 8, 2017, which are expressly incorporated by reference in their entirety, including the Figures, detailed description of the drawings, definitions, detailed description of the embodiments, and the examples, as well as all recited embodiments.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

What is claimed is:

1. An isolated cell comprising
a) a first polynucleotide encoding a first polypeptide comprising, from N- to C-terminal:
i) a first single domain antibody (sdAb) that binds to a human tumor target antigen (TTA);
ii) a first domain linker;
iii) a variable heavy chain comprising vhCDR1, vhCDR2, and vhCDR3;
iv) a first cleavable linker comprising a first protease cleavage site; and
v) a pseudo variable light chain; and
b) a second polynucleotide encoding a second polypeptide comprising, from N- to C-terminal:
i) a second sdAb that binds to a human tumor target antigen (TTA);
ii) a second domain linker;
iii) a variable light chain comprising vlCDR1, vlCDR2, and vlCDR3;
iv) a second cleavable linker comprising a second protease cleavage site; and
v) a pseudo variable heavy chain;
wherein said variable heavy chain of said first polypeptide and said variable light chain of said second polypeptide will bind human CD3 when associated to form a Fv.

2. The isolated cell according to claim 1, wherein said first sdAb and said second sdAb bind to the same human TTA.

3. The isolated cell according to claim 1 or 2, wherein said first sdAb and/or said second sdAb bind a human TTA selected from the group consisting of human EGFR, human B7H3, human EpCAM, and human FOLR1.

4. The isolated cell according to any one of claims 1-3, wherein said first sdAb and said second sdAb comprise the same amino acid sequence.

5. The isolated cell according to any one of claims 1-3, wherein said first sdAb and said second sdAb comprise different amino acid sequences.

6. The isolated cell according to any one of claims 1-5, wherein said first and second protease cleavage sites are recognized by the same protease.

7. The isolated cell according to any one of claims 1-5, wherein said first and second protease cleavage sites are recognized by different proteases.

8. The isolated cell according to any one of claims 1-7, wherein said first polypeptide further comprises a half life extension domain at the C-terminal end and/or said second polypeptide further comprises a half life extension domain at the C-terminal end.

9. The isolated cell according to any one of claims 1-8, wherein said variable heavy chain comprises the vhCDR1, vhCDR2, and vhCDR3 sequence of SEQ ID NO:102 of FIG. 39.

10. The isolated cell according to any one of claims 1-9, wherein said variable light chain comprises the vlCDR1, vlCDR2, and vlCDR3 sequence of SEQ ID NO:90 of FIG. 38.

11. The isolated cell according to any one of claims 1-10, wherein said pseudo variable heavy chain comprises the pseudo variable heavy chain sequence of any one selected from the group consisting of SEQ ID NO:106, SEQ ID NO:110 and SEQ ID NO: 207 of FIG. 39.

12. The isolated cell according to any one of claims 1-11 wherein said pseudo variable light chain comprises the pseudo variable light chain sequence of any one selected from the group consisting of SEQ ID NO:94, SEQ ID NO:98, and SEQ ID NO:203 of FIG. 38.

13. The isolated cell according to any one of claims 1-12, wherein said first polypeptide is selected from the group consisting of Pro16 (SEQ ID NO:5), Pro39 (SEQ ID NO:9), Pro41 (SEQ ID NO:13), Pro43 (SEQ ID NO:17), Pro45 (SEQ ID NO:21), and Pro349 (SEQ ID NO:25).

14. The isolated cell according to any one of claims 1-13, wherein said second polypeptide is selected from the group consisting of Pro19 (SEQ ID NO:7), Pro40 (SEQ ID NO:11), Pro42 (SEQ ID NO:15), Pro44 (SEQ ID NO:19), Pro46 (SEQ ID NO:23), and Pro353 (SEQ ID NO:27).

15. The isolated cell according to any one of claims 1-14, wherein said first polypeptide and said second polypeptide are selected from the group consisting of Pro16+Pro19, Pro39+Pro40, Pro41+Pro42, Pro43+Pro44, Pro45+Pro46, and Pro349+Pro353.

16. The isolated cell according to any one of claims 1-15, wherein said first polynucleotide and said second polynucleotide are introduced into said cell in different expression vectors.

17. The isolated cell according to any one of claims 1-15, wherein said first polynucleotide and said second polynucleotide are introduced into said cell in a single expression vector.

18. The isolated cell according to any one of claims 1-17, wherein said first polynucleotide and said second polynucleotide are introduced into said cell at a polynucleotide ratio to produce substantially equivalent amounts of said first polypeptide and said second polypeptide.

19. The isolated cell according to claim 18, wherein said ratio of said first polynucleotide to said second polynucleotide is 1:1.

20. The isolated cell according to claim 18, wherein said ratio of said first polynucleotide to said second polynucleotide is greater than 1:1.

21. The isolated cell according to claim 18, wherein said ratio of said first polynucleotide to said second polynucleotide is less than 1:1.

22. An expression vector comprising said first polynucleotide according to any one of claims 1-21.

23. An expression vector comprising said second polynucleotide according to any one of claims 1-21.

24. A composition comprising a first expression vector according to claim 22 and a second expression vector according to claim 23, wherein said first expression vector and said second expression vector are introduced into a host cell at a polynucleotide ratio to produce substantially equivalent amounts of said first polypeptide and said second polypeptide.

25. The composition according to claim 24, wherein said ratio of said first expression vector to said second expression vector is 1:1.

26. The composition according to claim 24, wherein said ratio of said first expression vector to said second expression vector is greater than 1:1.

27. The composition according to claim 24, wherein said ratio of said first expression vector to said second expression vector is less than 1:1.

28. A method of isolating a prodrug composition comprising a first polypeptide and a second polypeptide, the method comprising:
1) culturing a host cell under suitable culture conditions to produce and secrete a first polypeptide and a second polypeptide into culture media,
wherein said host cell comprises:
a) a first polynucleotide encoding said first polypeptide comprising, from N- to C-terminal:
i) a first sdAb that binds to a human tumor target antigen (TTA);
ii) a first domain linker;
iii) a variable heavy chain comprising vhCDR1, vhCDR2, and vhCDR3;
iv) a first cleavable linker comprising a first protease cleavage site; and
v) a pseudo variable light chain; and
b) a second polynucleotide sequence encoding a second polypeptide comprising, from N- to C-terminal:
i) a second sdAb that binds to a human tumor target antigen (TTA);
ii) a second domain linker;
iii) a variable light chain comprising vlCDR1, vlCDR2, and vlCDR3;
iv) a second cleavable linker comprising a second protease cleavage site; and
v) a pseudo variable heavy chain;
wherein said variable heavy chain of said first polypeptide and said variable light chain of said second polypeptide will bind human CD3 when associated to form an Fv; and
2) purifying said first polypeptide and said second polypeptide from said culture media using Protein A chromatography, thereby isolating a prodrug composition comprising a first polypeptide and a second polypeptide.

29. The method according to claim 28, wherein said first polypeptide and said second polypeptide are purified separately.

30. The method according to claim 28, wherein said first polypeptide and said second polypeptide are purified simultaneously.

31. The method according to any one of claims 28-30, wherein said purifying further comprises performing affinity chromatography after the Protein A chromatography.

32. The method according to any one of claims 28-31, wherein said prodrug composition comprises a substantially equivalent amount of said first polypeptide and said second polypeptide.

33. The method according to any one of claims 28-32, wherein said first sdAb and said second sdAb bind to the same human TTA.

34. The method according to any one of claims 28-33, wherein said first sdAb and/or said second sdAb bind a human TTA selected from the group consisting of human EGFR, human B7H3, human EpCAM, and human FOLR1.

35. The method according to any one of claims 28-34, wherein said first sdAb and said second sdAb comprise the same amino acid sequence.

36. The method according to any one of claims 28-34, wherein said first sdAb and said second sdAb comprise different amino acid sequences.

37. The method according to any one of claims 28-36, wherein said first and second protease cleavage sites are recognized by the same protease.

38. The method according to any one of claims 28-36, wherein said first and second protease cleavage sites are recognized by different proteases.

39. The method according to any one of claims 28-38, wherein said first polypeptide further comprises a half-life extension domain at the C-terminal end and/or said second polypeptide further comprises a half-life extension domain at the C-terminal end.

40. The method according to any one of claims 28-39, wherein said variable heavy chain comprises the vhCDR1, vhCDR2, and vhCDR3 sequence of SEQ ID NO:102 of FIG. 39.

41. The method according to any one of claims 28-40, wherein said variable light chain comprises the vlCDR1, vlCDR2, and vlCDR3 sequence of SEQ ID NO:90 of FIG. 38.

42. The method according to any one of claims 28-41, wherein said pseudo variable heavy chain comprises the pseudo variable heavy chain sequence of any one selected from the group consisting of SEQ ID NO:106, SEQ ID NO:110, and SEQ ID NO:207 of FIG. 39.

43. The method according to any one of claims 28-42, wherein said pseudo variable light chain comprises the pseudo variable light chain sequence of any one selected from the group consisting of SEQ ID NO:94, SEQ ID NO:98, and SEQ ID NO:203 of FIG. 38.

44. The method according to any one of claims 28-43, wherein said first polypeptide is selected from the group consisting of Pro16 (SEQ ID NO:5), Pro39 (SEQ ID NO:9), Pro41 (SEQ ID NO:13), Pro43 (SEQ ID NO:17), Pro45 (SEQ ID NO:21), and Pro349 (SEQ ID NO:25).

45. The method according to any one of claims 28-44, wherein said second polypeptide is selected from the group consisting of Pro19 (SEQ ID NO:7), Pro40 (SEQ ID NO:11), Pro42 (SEQ ID NO:15), Pro44 (SEQ ID NO:19), Pro46 (SEQ ID NO:23), and Pro353 (SEQ ID NO:27).

46. The method according to any one of claims 28-45, wherein said first polypeptide and said second polypeptide are selected from the group consisting of Pro16+Pro19, Pro39+Pro40, Pro41+Pro42, Pro43+Pro44, Pro45+Pro46, and Pro349+Pro353.

47. The method according to any one of claims 28-46, wherein said first polynucleotide and said second polynucleotide are introduced into said host cell in different expression vectors.

48. The method according to any one of claims 28-46, wherein said first polynucleotide and said second polynucleotide have been introduced into said host cell in a single expression vector.

49. The method according to any one of claims 28-48, said first polynucleotide and said second polynucleotide are introduced into said host cell at a polynucleotide ratio to produce substantially equivalent amounts said first polypeptide and said second polypeptide.

50. The method according to claim 49, wherein said polynucleotide ratio of said first polynucleotide to said second polynucleotide is 1:1.

51. The method according to claim 49, wherein said ratio of said first polynucleotide to said second polynucleotide is greater than 1:1.

52. The method according to claim 49, wherein said ratio of said first polynucleotide to said second polynucleotide is less than 1:1.

53. A method of treating cancer in a human subject in need thereof comprising administering the prodrug composition produced according to the method of any one of claims 28-52.
